SEQUENCE LISTING (68 sequences)

SEQ ID NO: 1 - protein, 199 aa, Cowpea chlorotic mottle virus
MSTVGTGKLTRAQRRAAARKNKRNTRVVQPVIVEPIASGQGRAIKAWTGYSVSKWTASCAAAEAWRAAAAKVTSAITISLPNELSSERNKQLKVGRVLLWLGLLPSVSGTVKSCVTETQTTAAASFQVALAVADNGILSKDVVAAMYPEAFKGITLEQLTADLTIYLYSSAALTEGDVIVHLEVEHVRPTFDDSFTPVY

SEQ ID NO: 2 - protein, 190 aa, Cowpea chlorotic mottle virus
MSTVGTGKLTRAQRRAAARKNKRNTRVVQPVIVEPIASGQGKAIKAWTGYSVSKWTASCAAAEAKVTSAITISLPNELSSERNKQLKVGRVLLWLGLLPSVSGTVKSCVTETQTTAAASFQVALAVADNSKDVVAAMYPEAFKGITLEQLTADLTIYLYSSAALTEGDVIVHLEVEHVRPTFDDSFTPVY

SEQ ID NO: 3 - DNA, 607 bp, Cowpea chlorotic mottle virus
ggactagtaggaggtaacttatgtcgaccgtgggtactgggaaattgactcgggcacaacgtcgtgctgcggcccgtaagaataagcgcaaaacccgcgtcgtccagcctgttatcgtcgagccaatcgcctcggggcaagggaaagccatcaaggcatggaccgggtactcggtgagcaaatggaccgcgtcgtgcgcggcagccgaggccaaagtgacgagcgcgatcaccatcagcttgcctaacgagctgtccagcgaacgcaacaagcagctcaaggtcggtcgtgtgctgctgtggttgggcctgctcccgagcgtctccggcaccgtgaagtcgtgcgtgacggaaacccagacgactgcggccgcatcgttccaagtggcgctcgccgtggccgataacagcaaggacgtggtggccgctatgtatcctgaggccttcaagggcatcaccctggagcagctgacggccgacctgacgatctacctgtactcctcggccgcgttgaccgagggcgatgtgatcgtgcacctcgaagttgaacacgtgcgcccgactttcgacgattcctttaccccggtttattgataatagctcgaggc

SEQ ID NO: 4 - DNA, 579 bp, Cowpea chlorotic mottle virus
atgtcgaccgtgggtactgggaaattgactcgggcacaacgtcgtgctgcggcccgtaagaataagcgcaaaacccgcgtcgtccagcctgttatcgtcgagccaatcgcctcggggcaagggaaagccatcaaggcatggaccgggtactcggtgagcaaatggaccgcgtcgtgcgcggcagccgaggccaaagtgacgagcgcgatcaccatcagcttgcctaacgagctgtccagcgaacgcaacaagcagctcaaggtcggtcgtgtgctgctgtggttgggcctgctcccgagcgtctccggcaccgtgaagtcgtgcgtgacggaaacccagacgactgcggccgcatcgttccaagtggcgctcgccgtggccgataacagcaaggacgtggtggccgctatgtatcctgaggccttcaagggcatcaccctggagcagctgacggccgacctgacgatctacctgtactcctcggccgcgttgaccgagggcgatgtgatcgtgcacctcgaagttgaacacgtgcgcccgactttcgacgattcctttaccccggtttattgataatag

SEQ ID NO: 5 - DNA, 579 bp, Cowpea chlorotic mottle virus
atgagtactgttggcactggtaaattgactcgggcccagcgtcgtgccgccgctcgcaagaataagcggaagacccgcgtggtccaacctgtgatcgtggagcccatcgcctccggccagggtaaagcgatcaaagcctggacggggtacagtgtcagcaaatggacggcttcgtgcgctgccgcggaggccaaggtcacgtccgctatcaccatttccctgcccaacgagctgagcagcgagcgcaataagcaactgaaggtcggccgggtcctgctgtggctggggttgttgcctagcgtgtcgggcaccgtgaagtcgtgcgtcaccgaaacccagaccacggcagccgcttcgttccaagtggcgctggccgtcgccgataattccaaggatgtcgtcgcggccatgtacccggaggcttttaagggcatcaccctggaacaattgaccgccgacctgactatctacctctattcgtcggctgccttgactgagggcgacgtgatcgtgcatttggaagtcgaacacgtccgtcctacctttgacgacagctttaccccggtgtactgataatag

SEQ ID NO: 6 - protein, 225 aa, Cowpea chlorotic mottle virus
MSTVGTGKLTRAQRRAAARKNKRNTRVVQPVIVEPIASGQGRAIKAWTGYSVSKWTASCAAAEAWRAAAAKVTSAITISLPNELSSERNKQLKVGRVLLWLGLLPSVSGTVKSCVTETQTTAAASFQVALAVADNGILSLLTEVETPIRNEWGCRCNDSSDGILSKDVVAAMYPEAFKGITLEQLTADLTIYLYSSAALTEGDVIVHLEVEHVRPTFDDSFTPVY

SEQ ID NO: 7 - protein, 217 aa, Cowpea chlorotic mottle virus
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNSRSLLTEVETPIRNEWGCRCNDSSDSRSKDVVAAMYPEAFKGITLEQLTADLTIYLYSSAALTEGDVIVHLEVEHVRPTFDDSFTPVY

SEQ ID NO: 8 - protein, 227 aa, Cowpea chlorotic mottle virus
MSTVGTGKLTRAQRRAAARKNKRNTRVVQPVIVEPIASGQGRAIKAWTGYSVSKWTASCAAAEAWRAAAAKVTSAITISLPNELSSERNKQLKVGRVLLWLGLLPSVSGTVKSCVTETQTTAAASFQVALAVADNGILSNSRKKRSTSAGPTVPDRDNDGIPDGILSKDVVAAMYPEAFKGITLEQLTADLTIYLYSSAALTEGDVIVHLEVEHVRPTFDDSFTPVY

SEQ ID NO: 9 - protein, 219 aa, Cowpea chlorotic mottle virus
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNSRSNSRKKRSTSAGPTVPDRDNDGIPDSRSKDVVAAMYPEAFKGITLEQLTADLTIYLYSSAALTEGDVIVHLEVEHVRPTFDDSFTPVY

SEQ ID NO: 10 - protein, 227 aa, Cowpea chlorotic mottle virus
MSTVGTGKLTRAQRRAAARKNKRNTRVVQPVIVEPIASGQGRAIKAWTGYSVSKWTASCAAAEAWRAAAAKVTSAITISLPNELSSERNKQLKVGRVLLWLGLLPSVSGTVKSCVTETQTTAAASFQVALAVADNGILRQDGKTFIDFKKYNDKLPLYISNPNGILSKDVVAAMYPEAFKGITLEQLTADLTIYLYSSAALTEGDVIVHLEVEHVRPTFDDSFTPVY

SEQ ID NO: 11 - protein, 219 aa, Cowpea chlorotic mottle virus
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNSRRQDGKTFIDFKKYNDKLPLYISNPNSRSKDVVAAMYPEAFKGITLEQLTADLTIYLYSSAALTEGDVIVHLEVEHVRPTFDDSFTPVY

SEQ ID NO: 12 - protein, 213 aa, Cowpea chlorotic mottle virus
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNSLLTEVETPIRNEWGCRCNDSSDSKDVVAAMYPEAFKGITLEQLTADLTIYLYSSAALTEGDVIVHLEVEHVRPTFDDSFTPVY

SEQ ID NO: 13 - protein, 215 aa, Cowpea chlorotic mottle virus
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNSNSRKKRSTSAGPTVPDRDNDGIPDSKDVVAAMYPEAFKGITLEQLTADLTIYLYSSAALTEGDVIVHLEVEHVRPTFDDSFTPVY

SEQ ID NO: 14 - protein, 215 aa, Cowpea Chlorotic Mottle Virus capsid protein containing a PA4 insert produced using SOE
MSTVGTGKLTRAQRRAAARKNKRNTRVVQPVIVEPIASGQGRAIKAWTGYSVSKWTASCAAAEAKVTSAITISLPNELSSERNKQLKVGRVLLWLGLLPSVSGTVKSCVTETQTTAAASFQVALAVADNRQDGKTFIDFKKYNDKLPLYISNPNSKDVVAAMYPEAFKGITLEQLTADLTIYLYSSAALTEGDVIVHLEVEHVRPTFDDSFTPVY

SEQ ID NO: 15 - DNA, 660 bp, Cowpea chlorotic mottle virus
atgtctacagtcggaacagggaagttaactcgtgcacaacgaagggctgcggcccgtaagaacaagcggaacactcgtgtggtccaacctgttattgtagaacccatcgcttcaggccaaggcaaggctattaaagcatggaccggttacagcgtatcgaagtggaccgcctcttgtgcggctgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaacgggatccgtcaagatggcaaaaccttcattgatttcaaaaagtataatgataaactccctctctatatttctaatcctaatgggatcctgtcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtat

SEQ ID NO: 16 - protein, 25 aa, Bacillus anthracis
SNSRKKRSTSAGPTVPDRDNDGIPD

SEQ ID NO: 17 - protein, 25 aa, Bacillus anthracis
SPEARHPLVAAYPIVHVDMENIILS

SEQ ID NO: 18 - protein, 25 aa, Bacillus anthracis
RIIFNGKDLNLVERRIAAVNPSDPL

SEQ ID NO: 19 - protein, 25 aa, Bacillus anthracis
RQDGKTFIDFKKYNDKLPLYISNPN

SEQ ID NO: 20 - protein, 25 aa, Eastern equine encephalomyelitis virus
DLDTHFTQYKLARPYIADCPNCGHS

SEQ ID NO: 21 - protein, 25 aa, Eastern equine encephalomyelitis virus
GRLPRGEGDTFKGKLHVPFVPVKAK

SEQ ID NO: 22 - protein, 76 aa, Artificial Sequence
  Chemically synthesized - Amino acid sequence of D2A21 trimer antimicrobial protective antigen (“PA”) peptide
FAKKFAKKFKKFAKKFAKFAFAFGDPFAKKFAKKFKKFAKKFAKFAFAFGDPFAKKFAKKFKKFAKKFAKFAFAFG

SEQ ID NO: 23 - DNA, 9200 bp, Artificial Sequence
  chemically synthesized - vector pDow1169 with CCMV CP inserted at SpeI, XhoI sites for expression in Pseudomonas fluorescens
cgggtacctgtcgaagggctggagacattcccggaaacgctgatgaagctgttcaacggcgagaacttcgggaagttggtgctcaaagtcagctgacacaccacaaaaacaaatgtgggagctggcttgcctgcgatgcaagcaactcggtttctaagtgataccgagttgatactatcgcaggcaagccagctcccacattttttgctcactctaaaaatcaggcgatctcggcgacgaccgctgccaacgcttttgcaggatccgccgcctggctgatcggccggccgatcaccaggtagtcagagcccgcatccagggcctggcgcggggtcaggatacggcgctggtcatcctgggcgctgccggtaggacggatacccggtgtcaccagttgcagcgacggatgtgcgtttttcagggcctgggcttccagggctgagcacaccaggccgtcgaggccggctttctgcgccagggctgccaggcgcaacacttgcacctgcggctcgatatccaggccaatgcccgccaggtcttcgcgctccatgctggtgagcacggtcacgccgatcaacaacggtttggggccgctgcgctgttccagcacttcgcggcaggcgctcatcatgcgcaggccaccggagcagtgcacattgaccatccacacgcccatctcggccgcggctttgacggccatcgccgtggtgttggggatgtcatggaatttgaggtcgaggaacacttcgaagcctttgtcccgcagggtgccgacgatttccgccgcgcaactggtgaacaattccttgccgaccttgacccggcaaagcttggggtccaactggtcagccagcttcagtgcggcgtcacgggtggggtaatccagggcgacgatgataggagtctggcagacggacattggaatgggctctcaggcaagtcgaaatcggcgcggattgtagcgcaagcagcgccgttgcgggatccgatgatcggtaaataccgatcaagcgcccaataccggcgattcaaggcaattgtgagcgctcacaatttattctgaaatgagctgttgacaattaatcatcggctcgtataatgtgtggaattgtgagcggataacaatttcacacaggaaacagaattttaatctactagtaggaggtaacttatgtccactgtcggcactggcaaattgacccgtgcacaacgtcgtgccgctgcccgcaagaacaagcggaagacccgggtggtccagccggtcatcgtggagccgattgcctccggccagggcaaggccattaaagcctggacgggctacagcgtcagcaaatggacggccagctgcgcggctgccgaggccaaggtcaccagtgcgattacgatcagcctccccaacgaattgagcagcgagcggaacaagcaactgaaggtcggccgggtcctgctgtggctgggtctgctgccgagtgtgtccggcaccgtcaagtcctgcgtgacggaaacccagactaccgcggcagcttcctttcaggttgccctggcagtggctgataactccaaagacgtcgttgcggccatgtacccagaggccttcaagggcatcaccctggagcagctgaccgcagacctgaccatttatctctacagcagtgccgcgctgacggaaggcgacgtcatcgtccatttggaagtggaacacgtccgcccgacgttcgatgactcgttcactccggtgtattgataatagctcgagcccaaaacgaaaggctcagtcgacagactgggcctttcgttttatctgttgtttgtcggtgaacgctctcctgagtaggacaaatccgccgggagcggatttgaacgttgcgaagcaacggcccggagggtggcgggcaggacgcccgccataaactgccaggcatcaaattaagcagaaggccatcctgacggatggcctttttgcgtttctacaaactcttttggcatatgggggatctggctgcaggagcagaagagcatacatctggaagcaaagccaggaaagcggcctatggagctgtgcggcagcgctcagtaggcaatttttcaaaatattgttaagccttttctgagcatggtatttttcatggtattaccaattagcaggaaaataagccattgaatataaaagataaaaatgtcttgtttacaatagagtggggggggtcagcctgccgccttgggccgggtgatgtcgtacttgcccgccgcgaactcggttaccgtccagcccagcgcgaccagctccggcaacgcctcgcgcacccgctggcggcgcttgcgcatggtcgaaccactggcctctgacggccagacatagccgcacaaggtatctatggaagccttgccggttttgccggggtcgatccagccacacagccgctggtgcagcaggcgggcggtttcgctgtccagcgcccgcacctcgtccatgctgatgcgcacatgctggccgccacccatgacggcctgcgcgatcaaggggttcagggccacgtacaggcgcccgtccgcctcgtcgctggcgtactccgacagcagccgaaacccctgccgcttgcggccattctgggcgatgatggataccttccaaaggcgctcgatgcagtcctgtatgtgcttgagcgccccaccactatcgacctctgccccgatttcctttgccagcgcccgatagctacctttgaccacatggcattcagcggtgacggcctcccacttgggttccaggaacagccggagctgccgtccgccttcggtcttgggttccgggccaagcactaggccattaggcccagccatggccaccagcccttgcaggatgcgcagatcatcagcgcccagcggctccgggccgctgaactcgatccgcttgccgtcgccgtagtcatacgtcacgtccagcttgctgcgcttgcgctcgccccgcttgagggcacggaacaggccgggggccagacagtgcgccgggtcgtgccggacgtggctgaggctgtgcttgttcttaggcttcaccacggggcacccccttgctcttgcgctgcctctccagcacggcgggcttgagcaccccgccgtcatgccgcctgaaccaccgatcagcgaacggtgcgccatagttggccttgctcacaccgaagcggacgaagaaccggcgctggtcgtcgtccacaccccattcctcggcctcggcgctggtcatgctcgacaggtaggactgccagcggatgttatcgaccagtaccgagctgccccggctggcctgctgctggtcgcctgcgcccatcatggccgcgcccttgctggcatggtgcaggaacacgatagagcacccggtatcggcggcgatggcctccatgcgaccgatgacctgggccatggggccgctggcgttttcttcctcgatgtggaaccggcgcagcgtgtccagcaccatcaggcggcggccctcggcggcgcgcttgaggccgtcgaaccactccggggccatgatgttgggcaggctgccgatcagcggctggatcagcaggccgtcagccacggcttgccgttcctcggcgctgaggtgcgccccaagggcgtgcaggcggtgatgaatggcggtgggcgggtcttcggcgggcaggtagatcaccgggccggtgggcagttcgcccacctccagcagatccggcccgcctgcaatctgtgcggccagttgcagggccagcatggatttaccggcaccaccgggcgacaccagcgccccgaccgtaccggccaccatgttgggcaaaacgtagtccagcggtggcggcgctgctgcgaacgcctccagaatattgataggcttatgggtagccattgattgcctcctttgcaggcagttggtggttaggcgctggcggggtcactacccccgccctgcgccgctctgagttcttccaggcactcgcgcagcgcctcgtattcgtcgtcggtcagccagaacttgcgctgacgcatccctttggccttcatgcgctcggcatatcgcgcttggcgtacagcgtcagggctggccagcaggtcgccggtctgcttgtccttttggtctttcatatcagtcaccgagaaacttgccggggccgaaaggcttgtcttcgcggaacaaggacaaggtgcagccgtcaaggttaaggctggccatatcagcgactgaaaagcggccagcctcggccttgtttgacgtataaccaaagccaccgggcaaccaatagcccttgtcacttttgatcaggtagaccgaccctgaagcgcttttttcgtattccataaaacccccttctgtgcgtgagtactcatagtataacaggcgtgagtaccaacgcaagcactacatgctgaaatctggcccgcccctgtccatgcctcgctggcggggtgccggtgcccgtgccagctcggcccgcgcaagctggacgctgggcagacccatgaccttgctgacggtgcgctcgatgtaatccgcttcgtggccgggcttgcgctctgccagcgctgggctggcctcggccatggccttgccgatttcctcggcactgcggccccggctggccagcttctgcgcggcgataaagtcgcacttgctgaggtcatcaccgaagcgcttgaccagcccggccatctcgctgcggtactcgtccagcgccgtgcgccggtggcggctaagctgccgctcgggcagttcgaggctggccagcctgcgggccttctcctgctgccgctgggcctgctcgatctgctggccagcctgctgcaccagcgccgggccagcggtggcggtcttgcccttggattcacgcagcagcacccacggctgataaccggcgcgggtggtgtgcttgtccttgcggttggtgaagcccgccaagcggccatagtggcggctgtcggcgctggccgggtcggcgtcgtactcgctggccagcgtccgggcaatctgcccccgaagttcaccgcctgcggcgtcggccaccttgacccatgcctgatagttcttcgggctggtttccactaccagggcaggctcccggccctcggctttcatgtcatccaggtcaaactcgctgaggtcgtccaccagcaccagaccatgccgctcctgctcggcgggcctgatatacacgtcattgccctgggcattcatccgcttgagccatggcgtgttctggagcacttcggcggctgaccattcccggttcatcatctggccggtggtggcgtccctgacgccgatatcgaagcgctcacagcccatggccttgagctgtcggcctatggcctgcaaagtcctgtcgttcttcatcgggccaccaagcgcagccagatcgagccgtcctcggttgtcagtggcgtcaggtcgagcaagagcaacgatgcgatcagcagcaccaccgtaggcatcatggaagccagcatcacggttagccatagcttccagtgccacccccgcgacgcgctccgggcgctctgcgcggcgctgctcacctcggcggctacctcccgcaactctttggccagctccacccatgccgcccctgtctggcgctgggctttcagccactccgccgcctgcgcctcgctggcctgctgggtctggctcatgacctgccgggcttcgtcggccagtgtcgccatgctctgggccagcggttcgatctgctccgctaactcgttgatgcctctggatttcttcactctgtctattgcgttcgtggtctattgcgttcgtggtctattgcctcccggtattcctgtaagtcgatgatctgggcgttggcggtgtcgatgttcagggccacgtctgcccggtcggtgcggatgccccggccttccatctccaccacgttcggccccaggtgaacaccgggcaggcgctcgatgccctgcgcctcaagtgttctgtggtcaatgcgggcgtcgtggccagcccgctctaatgcccggttggcatggtcggcccatgcctcgcgggtctgctcaagccatgccttgggcttgagcgcttcggtcttctgtgccccgcccttctccggggtcttgccgttgtaccgcttgaaccactgagcggcgggccgctcgatgccgtcattgatccgctcggagatcatcaggtggcagtgcgggttctcgccgccaccggcatggatggccagcgtatacggcaggcgctcggcaccggtcaggtgctgggcgaactcggacgccagcgccttctgctggtcgagggtcagctcgaccggcagggcaaattcgacctccttgaacagccgcccattggcgcgttcatacaggtcggcagcatcccagtagtcggcgggccgctcgacgaactccggcatgtgcccggattcggcgtgcaagacttcatccatgtcgcgggcatacttgccttcgcgctggatgtagtcggccttggccctggccgattggccgcccgacctgctgccggttttcgccgtaaggtgataaatcgccatgctgcctcgctgttgcttttgcttttcggctccatgcaatggccctcggagagcgcaccgcccgaagggtggccgttaggccagtttctcgaagagaaaccggtaagtgcgccctcccctacaaagtagggtcgggattgccgccgctgtgcctccatgatagcctacgagacagcacattaacaatggggtgtcaagatggttaaggggagcaacaaggcggcggatcggctggccaagctcgaagaacaacgagcgcgaatcaatgccgaaattcagcgggtgcgggcaagggaacagcagcaagagcgcaagaacgaaacaaggcgcaaggtgctggtgggggccatgattttggccaaggtgaacagcagcgagtggccggaggatcggctcatggcggcaatggatgcgtaccttgaacgcgaccacgaccgcgccttgttcggtctgccgccacgccagaaggatgagccgggctgaatgatcgaccgagacaggccctgcggggctgcacacgcgcccccacccttcgggtagggggaaaggccgctaaagcggctaaaagcgctccagcgtatttctgcggggtttggtgtggggtttagcgggctttgcccgcctttccccctgccgcgcagcggtggggcggtgtgtagcctagcgcagcgaatagaccagctatccggcctctggccgggcatattgggcaagggcagcagcgccccacaagggcgctgataaccgcgcctagtggattattcttagataatcatggatggatttttccaacaccccgccagcccccgcccctgctgggtttgcaggtttgggggcgtgacagttattgcaggggttcgtgacagttattgcaggggggcgtgacagttattgcaggggttcgtgacagttagtacgggagtgacgggcactggctggcaatgtctagcaacggcaggcatttcggctgagggtaaaagaactttccgctaagcgatagactgtatgtaaacacagtattgcaaggacgcggaacatgcctcatgtggcggccaggacggccagccgggatcgggatactggtcgttaccagagccaccgacccgagcaaacccttctctatcagatcgttgacgagtattacccggcattcgctgcgcttatggcagagcagggaaaggaattgccgggctatgtgcaacgggaatttgaagaatttctccaatgcgggcggctggagcatggctttctacgggttcgctgcgagtcttgccacgccgagcacctggtcgctttcagctgtaagcgtcgcggtttctgcccgagctgtggggcgcggcggatggccgaaagtgccgccttgctggttgatgaagtactgcctgaacaacccatgcgtcagtgggtgttgagcttcccgtttcagctgcgtttcctgtttggggtcgtttgcgggaaggggcggaatcctacgctaaggctttggccagcgatattctccggtgagattgatgtgttcccaggggataggagaagtcgcttgatatctagtatgacgtctgtcgcacctgcttgatcgcggcccaccgcggcgggaagcaggtgcgattttcgcgaaggcatgcccgtcaccacgtcgaaaaacaaaatcaccagattctccgcctctgacaggcaaccagtcagaatgcgattcaccaaaaaaaatattagttcgattcaatggaggttccttcagttttctgatgaagcgcgaatatagagaaatatcccgaatgtgcagttaacgaattccggctgtccggcgttttcgtggagcccgaacagcgaggccgaggggtcgccggtatgctgctgcgggcgttgccggcgggtttattgctcgtgatgatcgtccgacagattccaacgggaatctggtggatgcgcatcttcatcctcggcgcacttaatatttcgctattctggagcttgttgtttatttcggtctaccgcctgccgggcggggtcgcggcgacggtaggcgctgtgcagccgctgatggtcgtgttcatctctgccgctctgctaggtagcccgatacgattgatggcggtcctgggggctatttgcggaactgcgggcgtggcgctgttggtgttgacaccaaacgcagcgctagatcctgtcggcgtcgcagcgggcctggcgggggcggtttccatggcgttcggaaccgtgctgacccgcaagtggcaacctcccgtgcctctgctcacctttaccgcctggcaactggcggccggaggacttctgctcgttccagtagctttagtgtttgatccgccaatcccgatgcctacaggaaccaatgttctcggcctggcgtggctcggcctgatcggagcgggtttaacctacttcctttggttccgggggatctcgcgactcgaacctacagttgtttccttactgggctttctcagcccggggaccgccgtgttgctaggatggttgttcttggatcagacgctgagtgcgcttcaaatcatcggcgtcctgctcgtgatcgggagtgtctggctgggccaacgttccaaccgcactcctagggcgcgtatagcttgccggaagtcgccttgacccgcatggcataggcctatcgtttccacgatcagcgat

SEQ ID NO: 24 - DNA, 36 bp, Cowpea chlorotic mottle virus
tgcgcggctgccgagagcgctgccaaggtcaccagt

SEQ ID NO: 25 - DNA, 36 bp, Cowpea chlorotic mottle virus
actggtgaccttggcagcgctctcggcagccgcgca

SEQ ID NO: 26 - DNA, 36 bp, Cowpea chlorotic mottle virus
ctgccgagtgtgtcccgatcgggcaccgtcaagtcc

SEQ ID NO: 27 - DNA, 36 bp, Cowpea chlorotic mottle virus
ggacttgacggtgcccgatcgggacacactcggcag

SEQ ID NO: 28 - DNA, 36 bp, Cowpea chlorotic mottle virus
acggaaacccagactagatctaccgcggcagcttcc

SEQ ID NO: 29 - DNA, 36 bp, Cowpea chlorotic mottle virus
ggaagctgccgcggtagatctagtctgggtttccgt

SEQ ID NO: 30 - DNA, 36 bp, Cowpea chlorotic mottle virus
gcagtggctgataactcaagatccaaagacgtcgtt

SEQ ID NO: 31 - DNA, 36 bp, Cowpea chlorotic mottle virus
aacgacgtctttggatcttgagttatcagccactgc

SEQ ID NO: 32 - DNA, 38 bp, Cowpea chlorotic mottle virus
ccatttatctctacagcgctagcagtgccgcgctgacg

SEQ ID NO: 33 - DNA, 38 bp, Cowpea chlorotic mottle virus
cgtcagcgcggcactgctagcgctgtagagataaatgg

SEQ ID NO: 34 - protein, 23 aa, Artificial Sequence
  Chemically synthesized - Amino acid sequence of flu vaccine M2e 1 peptide
SLLTEVETPIRNEWGCRCNDSSD

SEQ ID NO: 35 - DNA, 69 bp, Artificial Sequence
  Chemically synthesized - Codon optimized nucleic acid sequence for M2e 1
agcttgttgactgaagttgaaacgccaatccgtaatgaatggggctgccggtgcaacgatagttccgac

SEQ ID NO: 36 - DNA, 52 bp, Artificial Sequence
  Chemically synthesized - Primers for cloning into the restriction site XbaI in the 129 surface loop M2e CCMV129 F
cgtctagaagcttgttgactgaagttgaaacgccaatccgtaatgaatgggg

SEQ ID NO: 37 - DNA, 52 bp, Artificial Sequence
  Chemically synthesized - Primers for cloning into the restriction site XbaI in the 129 surface loop M2e CCMV129 R
cgtctagagtcggaactatcgttgcaccggcagccccattcattacggattg

SEQ ID NO: 38 - DNA, 40 bp, Artificial Sequence
  Chemically synthesized - Primers for SOE based M2e CCMV fusion Coop CCMV F
ggactagtaggaggtaacttatgtccactgtcggcactgg

SEQ ID NO: 39 - DNA, 35 bp, Artificial Sequence
  Chemically synthesized - Primers for SOE based M2e CCMV fusion Coop CCMV R
ccgctcgagtcattactattatcaatacaccggag

SEQ ID NO: 40 - DNA, 63 bp, Artificial Sequence
  Chemically synthesized - Primers for SOE based M2e CCMV fusion M2e CCMV129 SOE F
caatccgtaatgaatggggctgccggtgcaacgatagttccgactccaaagacgtcgttgcgg

SEQ ID NO: 41 - DNA, 71 bp, Artificial Sequence
  Chemically synthesized - Primers for SOE based M2e CCMV fusion M2e CCMV129 SOE R
ccccattcattacggattggcgtttcaacttcagtcaacaagcttctagacggttatcagccactgccagg

SEQ ID NO: 42 - DNA, 45 bp, Artificial Sequence
  Chemically synthesized - M2e 1 blunt F, primers to synthesize blunt ended M2e 1 DNA fragment
tcactcttgacagaggtagaaacaccgatacgtaatgaatggggc

SEQ ID NO: 43 - DNA, 45 bp, Artificial Sequence
  Chemically synthesized - M2e 1 blunt R, primers to synthesize blunt ended M2e 1 DNA fragment
atctgaagaatcattacaacgacagccccattcattacgtatcgg

SEQ ID NO: 44 - DNA, 69 bp, Cowpea chlorotic mottle virus
ccagcgccggtccaaccgtgcccgaccgcgacaacgatggcatccccgactccaaagacgtcgttgcgg

SEQ ID NO: 45 - DNA, 69 bp, Cowpea chlorotic mottle virus
tcgggcacggttggaccggcgctggtggagcgtttcttgcgactattactgttatcagccactgccagg

SEQ ID NO: 46 - protein, 25 aa, Artificial Sequence
  Chemically synthesized - anthrax vaccine peptide PA1
SNSRKKRSTSAGPTVPDRDNDGIPD

SEQ ID NO: 47 - DNA, 75 bp, Artificial Sequence
  Chemically synthesized - Codon optimized nucleic acid sequence for anthrax vaccine peptide PA1
agtaatagtcgcaagaaacgctccaccagcgccggtccaaccgtgcccgaccgcgacaacgatggcatccccgac

SEQ ID NO: 48 - DNA, 58 bp, Artificial Sequence
  Chemically synthesized - PA1 DNA insert synthesized by PCR using PA1 129 XbaI F
cttctagaagtaatagtcgcaagaaacgctccaccagcgccggtccaaccgtgcccga

SEQ ID NO: 49 - DNA, 58 bp, Artificial Sequence
  Chemically synthesized - PA1 DNA insert synthesized by PCR using PA1 129 XbaI R
cttctagagtcggggatgccatcgttgtcgcggtcgggcacggttggaccggcgctgg

SEQ ID NO: 50 - protein, 25 aa, Artificial Sequence
  Chemically synthesized - Peptide sequence of anthrax vaccine peptide PA4
RQDGKTFIDFKKYNDKLPLYISNPN

SEQ ID NO: 51 - DNA, 75 bp, Artificial Sequence
  Chemically synthesized - Codon optimized nucleic acid sequence of anthrax vaccine peptide PA4
cgccaggatggtaagacgttcatcgactttaagaaatacaacgacaagctgcccctgtatatttccaaccctaat

SEQ ID NO: 52 - DNA, 58 bp, Artificial Sequence
  Chemically synthesized - The PA4 DNA insert is synthesized by PCR using primers PA4 129 XbaI F
cttctagacgccaggatggtaagacgttcatcgactttaagaaatacaacgacaagct

SEQ ID NO: 53 - DNA, 58 bp, Artificial Sequence
  Chemically synthesized - The PA4 DNA insert is synthesized by PCR using primers PA4 129 XbaI R
cttctagaattagggttggaaatatacaggggcagcttgtcgttgtatttcttaaagt

SEQ ID NO: 54 - DNA, 69 bp, Artificial Sequence
  Chemically synthesized - anthrax vaccine peptide PA4 fused to codon and hydrophilicity optimized CCMV capsid protein PA4 129 SOE F
actttaagaaatacaacgacaagctgcccctgtatatttccaaccctaattccaaagacgtcgttgcgg

SEQ ID NO: 55 - DNA, 69 bp, Artificial Sequence
  Chemically synthesized - anthrax vaccine peptide PA4 fused to codon and hydrophilicity optimized CCMV capsid protein PA4 129 SOE R
agcttgtcgttgtatttcttaaagtcgatgaacgtcttaccatcctggcggttatcagccactgccagg

SEQ ID NO: 56 - DNA, 35 bp, Cowpea chlorotic mottle virus
ctactagtaggaggtaacttatgtctacagtcgga

SEQ ID NO: 57 - DNA, 29 bp, Cowpea chlorotic mottle virus
ccgctcgagtcattaatacaccggagtga

SEQ ID NO: 58 - DNA, 663 bp, Cowpea chlorotic mottle virus
atgtctacagtcggaacagggaagttaactcgtgcacaacgaagggctgcggcccgtaagaacaagcggaacactcgtgtggtccaacctgttattgtagaacccatcgcttcaggccaaggcaaggctattaaagcatggaccggttacagcgtatcgaagtggaccgcctcttgtgcggctgccgaagctaaagtaacctcggctataactatctctctccctaatgagctatcgtccgaaaggaacaagcagctcaaggtaggtagagttttattatggcttgggttgcttcccagtgttagtggcacagtgaaatcctgtgttacagagacgcagactactgctgctgcctcctttcaggtggcattagctgtggccgacaacgggatccttagtaattctcgtaagaaacgttctacctctgctggccctaccgtgcctgatcgtgataatgatggcattcctgatgggatcctgtcgaaagatgttgtcgctgctatgtaccccgaggcgtttaagggtataacccttgaacaactcaccgcggatttaacgatctacttgtacagcagtgcggctctcactgagggcgacgtcatcgtgcatttggaggttgagcatgtcagacctacgtttgacgactctttcactccggtgtat

SEQ ID NO: 59 - DNA, 41 bp, Cowpea chlorotic mottle virus
gcattagctgtggccgacaacagtaattctcgtaagaaacg

SEQ ID NO: 60 - DNA, 41 bp, Cowpea chlorotic mottle virus
cgtttcttacgagaattactgttgtcggccacagctaatgc

SEQ ID NO: 61 - DNA, 44 bp, Cowpea chlorotic mottle virus
cgtgataatgatggcattcctgattcgaaagatgttgtcgctgc

SEQ ID NO: 62 - DNA, 44 bp, Cowpea chlorotic mottle virus
gcagcgacaacatctttcgaatcaggaatgccatcattatcacg

SEQ ID NO: 63 - DNA, 42 bp, Cowpea chlorotic mottle virus
gcattagctgtggccgacaaccgtcaagatggcaaaaccttc

SEQ ID NO: 64 - DNA, 42 bp, Cowpea chlorotic mottle virus
gaaggttttgccatcttgacggttgtcggccacagctaatgc

SEQ ID NO: 65 - DNA, 41 bp, Cowpea chlorotic mottle virus
ctctatatttctaatcctaattcgaaagatgttgtcgctgc

SEQ ID NO: 66 - DNA, 41 bp, Cowpea chlorotic mottle virus
gcagcgacaacatctttcgaattaggattagaaatatagag

SEQ ID NO: 67 - DNA, 579 bp, Cowpea chlorotic mottle virus; identical to SEQ ID NO: 4

SEQ ID NO: 68 - DNA, 579 bp, Cowpea chlorotic mottle virus; identical to SEQ ID NO: 5

(19) United States
(12) Patent Application Publication
(10) Pub. No.: US 2009/0093019 A1
(43) Pub. Date: Apr. 9, 2009

(54) PRODUCTION AND IN VIVO ASSEMBLY OF SOLUBLE RECOMBINANT ICOSAHEDRAL VIRUS-LIKE PARTICLES

(76) Inventors: Jamie P. Phelps, Aurora, CO (US); Lada Rasochova, Del Mar, CA (US)

Correspondence Address: TRASKBRITT, P.C.\Dow Global Technologies Inc., PO Box 2550, SALT LAKE CITY, UT, 84110, US

(21) Appl. No.: 12/110,257

(22) Filed: Apr. 25, 2008

Related U.S. Application Data
(60) Provisional application No. 60/914,677, filed on Apr. 27, 2007.

(51) Int. Cl. C12P 21/04

(52) U.S. Cl. 435/69.1

(57) ABSTRACT

The present invention provides an improved method for the in vivo production of soluble assembled virus-like particles (“VLPs”) in bacterial cells of Pseudomonad origin. The Pseudomonad cells support assembly of VLPs from icosahedral viral capsid proteins (“CPs”) in vivo, and allow the inclusion of larger recombinant peptides as monomers or concatamers in the VLP. The invention specifically provides an improved method for the in vivo production of soluble assembled Cowpea Chlorotic Mottle Virus (“CCMV”) VLPs by introducing modifications into the CCMV CP that result in high yield production of soluble CP fusions in a Pseudomonas fluorescens bacterial system. These soluble VLPs can subsequently be purified and used as vaccines.

### CROSS-REFERENCE TO RELATED APPLICATION

This application is a utility conversion of U.S. Provisional Patent Application Ser. No. 60/914,677, filed Apr. 27, 2007, the entire contents of which are hereby incorporated herein by this reference.

### STATEMENT OF GOVERNMENT INTEREST

This invention was made with government support under a United States Government contract with the National Institutes of Health, National Institute of Allergy and Infectious Disease (NIAID), Cooperative Agreement No. 1-U01-AI054641-01. The government has certain rights to this invention.

### FIELD OF THE INVENTION

The present invention provides an improved method for the production of soluble, assembled virus-like particles (“VLPs”) in a bacterial host cell.

### BACKGROUND OF THE INVENTION

Bacterial, yeast, Dictyostelium discoideum, insect, and mammalian cell expression systems are currently used to produce recombinant peptides for use as human and animal therapeutics, with varying degrees of success. One goal in creating expression systems for the production of heterologous peptides is to provide broad based, flexible, efficient, economic, and practical platforms and methods that can be utilized in commercial, therapeutic, and vaccine applications. For example, the production of certain polypeptides, it would be desirable to provide an expression system capable of producing, in an efficient and inexpensive manner, large quantities of soluble, desirable products in vivo in order to eliminate or reduce downstream reassembly costs.

Currently, bacteria are the most widely used expression system for the production of recombinant peptides because of their potential to produce abundant quantities of recombinant peptides. However, bacteria are often limited in their capacities to produce certain types of peptides, requiring the use of alternative, and more expensive, expression systems. For example, bacterial systems are restricted in their capacity to produce monomeric antimicrobial peptides due to the toxicity of such peptides to the bacteria, often leading to the death of the cell upon the expression of the peptide. Because of the inherent disadvantages of non-bacterial expression systems, significant time and resources have been spent on trying to improve the capacity of bacterial systems to produce a wide range of commercially and therapeutically useful peptides. While progress has been made in this area, additional methods and platforms for the production of heterologous peptides in bacterial expression systems would be beneficial.

### Viruses

One approach for improving peptide production in host cell expression systems includes use of replicative viruses to produce recombinant polypeptides of interest. However, the use of replicative, full-length viruses has numerous drawbacks for use in recombinant polypeptide production strategies. For example, it may be difficult to control recombinant polypeptide production during fermentation conditions, which may require tight regulation of expression in order to maximize efficiency of the fermentation run. Furthermore, the use of replicative viruses to produce recombinant polypeptides may result in the imposition of regulatory requirements, which may lead to increased downstream purification steps.

To overcome production issues, particularly during fermentation, one area of research has focused on the expression and assembly of viruses in a cell that is not a natural host to the particular virus (a non-tropic host cell). A non-tropic cell is a cell that the virus is incapable of successfully entering due to incompatibility between virus capsid proteins and the host receptor molecules, or an incompatibility between the biochemistry of the virus and the biochemistry of the cell, thereby preventing the virus from completing its life cycle. For example, U.S. Pat. No. 5,869,287 to Price et al. describes a method for synthesizing and assembling, in yeast cells, replicable or infectious viruses containing RNA, where either the viral capsid proteins or the RNA contained within the capsids are from a non-yeast virus species of Nodaviridae or Bromoviridae. This approach, however, does not overcome the potential regulatory hurdles that are associated with protein production in replicative viruses.

### Virus-Like Particles (VLPs)

Another approach for improving the production of recombinant peptides has been to use VLPs. The particulate nature of VLPs generally induce a more effective immune response than denatured proteins or soluble proteins. VLPs have a number of advantages over conventional immunogens as vaccines. Antigens from various infectious agents, for example, can be synthesized as VLPs in heterologous expression systems. In addition to the ability of certain capsid or envelope proteins to self-assemble, these particles can be produced in large quantities, and are easily enriched and purified. Vaccination with chimeric VLPs can induce both insert-specific B and/or T-cell responses even in the absence of adjuvant; furthermore, VLPs cannot replicate and are non-infectious.

In general, encapsidated viruses include a protein coat or “capsid” that is assembled to contain the viral nucleic acid. Many viruses have capsids that can be “self-assembled” from the individually expressed capsid proteins—both within the cell the capsid is expressed in (“in vivo assembly”) forming VLPs, and outside of the cell after isolation and purification (“in vitro assembly”).

### Use of Virus-Like Particles in Bacterial Expression Systems

Ideally, capsid proteins (“CPs”) are modified to contain a target recombinant polypeptide, generating a recombinant viral CP-peptide fusion. The fusion peptide can then be expressed in a cell, and, ideally, assembled in vivo to form recombinant VLPs in a soluble form. Because of the potential of fast, efficient, inexpensive, and abundant yields of recombinant polypeptides, bacteria have been examined as host cells in expression systems for the production of assembled, soluble recombinant viral CP-peptide fusion VLPs.

Researchers have shown that particular wild-type (“wt”) viral capsid proteins without recombinant polypeptide inserts can be transgenically expressed in non-tropic enterobacteria. Researchers have also shown that these capsid proteins can be assembled, both in vivo and in vitro, to form VLPs. See, for example, S. J. Shire et al., Biochemistry 29(21):5119-26 (29 May 1990) (in vitro assembly of virus-like particles from helical tobacco mosaic virus capsid proteins expressed in E. coli); X. Zhao et al., Virology 207(2):486-94 (10 Mar. 1995) (in vitro assembly of virus-like particles from icosahedral cowpea chlorotic mottle virus capsid proteins expressed in E. coli); Y. Stram et al., Virus Res. 28(1):29-35 (April 1993) (expression of filamentous potato virus Y capsid proteins in E. coli, with in vivo formation of virus-like particles); J. Joseph and H. S. Savithri, Arch. Virol. 144(9): 1679-87 (1999) (expression of filamentous chili pepper vein banding virus capsid proteins in E. coli, with in vivo formation of virus-like particles); D. J. Hwang et al., Proc. Nat'l Acad. Sci. USA 91(19):9067-71 (13 Sep. 1994) (expression of helical tobacco mosaic virus capsid proteins in E. coli, with in vivo formation of virus-like particles); M. Sastri et al., J. Mol. Biol. 272(4):541-52 (3 Oct. 1997) (expression of icosahedral physalis mottle virus capsid proteins in E. coli, with in vivo formation of virus-like particles).

To date, successful expression and in vivo assembly of recombinant viral CP-peptide fusion particles in a non-tropic bacterial cell has been varied. Brumfield et al., for example, unsuccessfully attempted to express as in vivo assembled VLPs recombinant polypeptides inserted into an icosahedral capsid protein. See Brumfield et al. (2004) “Heterologous expression of the modified capsid protein of Cowpea chlorotic mottle bromovirus results in the assembly of protein cages with altered architectures and functions,” J. Gen. Vir. 85:1049-1053. The reasons for the observed inability of icosahedral viral CP-peptide fusion particles to assemble as VLPs in vivo in E. coli has not been well understood.

U.S. patent application Ser. No. 11/001,626 describes a method for the production of in vivo assembled VLPs containing peptide inserts in the bacterial host cell Pseudomas fluorescens. A cowpea chlorotic mottle bromovirus capsid protein was described that had been engineered to contain restriction enzyme digestion sites at the peptide insertion site to allow insertion of a peptide of interest.

Further improvements in the production of VLPs containing inserted peptides of interest can allow for increased yields of soluble, assembled VLPs in vivo. The production of higher yields of soluble VLPs can allow for a reduction in processing steps due to the decreased need to solubilize, denature, renature, properly refold and assemble previously insoluble VLPs. Increased yields of soluble, assembled VLPs in vivo can thus make the manufacturing process more efficient.

### SUMMARY OF THE INVENTION

The present invention provides nucleic acid constructs and methods of use thereof for the production of soluble, in vivo assembled virus like particles (VLPs) in bacterial host cells. The nucleic acid constructs are engineered to optimize the hydrophilicity of a viral capsid protein (CP) or CP-peptide fusion using a set of hydrophilicity-optimization rules. The hydrophilicity optimized nucleic acid constructs are designed, through the removal, mutagenesis, or addition of certain codons in focused area identified by the hydrophilicity optimization rules to allow for an increase in the yield of soluble VLPs assembled in vivo.

In some embodiments of the present invention, a low hydrophilicity value area can be increased by removing codons encoding amino acids that have an undesirably low hydrophilicity value. In other embodiments of the invention, the inserted peptide can be modified by removing amino acids at position 63 and 129 insertion sites of the original CCMV coat protein construct by site directed mutagenesis or using splicing by overlap extension (“SOE”)-based technology.

In additional embodiments of the present invention, the hydrophilicity value of an identified area having a low hydrophilicity value can be increased by replacing a codon encoding an amino acid of low hydrophilicity with an amino acid having a higher hydrophilicity value. Alternatively, the hydrophilicity of a focused area can be increased by adding one or more than one codons encoding amino acids with desirable hydrophilicity values.

In some embodiments, the present invention provides isolated nucleic acid constructs encoding a hydrophilicity-optimized viral capsid protein. In one embodiment, the hydrophilicity-optimized capsid protein is derived from an icosahedral virus. In one embodiment, the icosahedral virus is CCMV. In one embodiment, the viral capsid protein is derived from SEQ ID NO:1.

In other embodiments, the present invention provides an isolated nucleic acid construct encoding a viral capsid protein, wherein the nucleic acid construct contains an engineered restriction site encoding an area of hydrophilicity of at least 50%. The engineered restriction site provides an insertion site for a peptide of interest, allowing the production of viral capsid protein-peptide fusion peptides (CP-peptide fusions) that can self-assemble into soluble VLPs. In some embodiments, the restriction site has an area of hydrophilicity of at least 50%, at least 55%, at least 60%, at least 65%, at least 70%, at least 75%, at least 80%, or at least 85%. In additional embodiments, the engineered restriction site has an area of hydrophilicity of at least 75%. In other embodiments, the engineered restriction site is comprised of nucleic acid codons encoding the amino acids Aspartic Acid, Glutamic Acid, Lysine, or Arginine (Asp-Glu-Lys-Arg). In some embodiments, the engineered restriction site does not contain codons encoding two or more consecutive hydrophobic amino acids selected from the group consisting of Alanine, Phenylalanine, Tryptophan, Tryptophan, Valine, Leucine, Methionine, or Proline. In yet other embodiments, the engineered restriction site is contained in a CCMV capsid protein. In some embodiments, the hydrophilicity-optimized nucleic acid construct encoding a viral capsid protein having an engineered restriction site is selected from the group consisting of SEQ ID NOS:3, 4 and 5.

In alternative embodiments of the present invention, hydrophilicity-optimized nucleic acid constructs are provided encoding CP-peptide fusions. In some embodiments, the peptide insert is altered to increase the hydrophilicity of the peptide. In other embodiments, the hydrophilicity is altered to provide a recombinant peptide comprising less than 50% hydrophobic amino acids. In some embodiments, the peptide is altered to comprise a hydrophilicity of at least 50%, at least 55%, at least 56%, at least 60%, at least 65%, at least 70%, or at least 75%. The hydrophilicity of a peptide insert can be altered by the addition or subtraction of codons encoding amino acids from the N- or C-terminus of the peptide. In some embodiments, the hydrophilicity of the peptide insert can be increased so that the hydrophilicity of the peptide insert is at least 56%. In some embodiments, the hydrophilicity of the insert is increased by adding at least one hydrophilic amino acid to the N- or C-terminus of the peptide, wherein the amino acid is selected from an amino acid having a hydrophilicity value of greater than one (1), as determined by a modified Roseman hydrophobicity scale (Table 1). In additional embodiments, the hydrophilicity-optimized amino acid sequence is selected from the group consisting of SEQ ID NOS:7, 9, 11, 12, 13 and 14.

The present invention further includes bacterial cells comprising nucleic acid constructs engineered to optimize the hydrophilicity of a viral capsid protein (CP) or CP-peptide fusion using a set of hydrophilicity-optimization rules. In some embodiments, the cell produces soluble assembled recombinant virus-like particles in vivo. In other embodiments, the cell of the present invention provide from 0.5 g/L, 1.0 g/L, 1.5 g/L, 2 g/L or more than 2 g/L of soluble, assembled VLPs when expressed from the hydrophilicity optimized nucleic acid construct. In some embodiments, the bacterial host cell is a Pseudomonad such as Pseudomonas fluorescens.

### BRIEF DESCRIPTION OF THE FIGURES

FIG. 1 presents a plasmid map of a CCMV129-CP expression plasmid useful for expression of recombinant VLPs in Pseudomonad host cells. The CCMV CP has not been hydrophilicity optimized.

FIG. 2 illustrates a scheme for production of peptide monomers in Virus-Like Particles (“VLP”) in host cells, e.g., Pseudomonad host cells. A desired target peptide insert coding sequence (“I”) is inserted, in-frame, into the viral capsid coding sequence (“CP”) in constructing a recombinant viral capsid gene (“rCP”), which, as part of a vector, is transformed into the host cell and expressed to form recombinant capsids (“rCP”). These are then assembled to form VLPs containing up to 180 rCPs each, in the case of CCMV. The VLPs are illustrated with target peptide inserts (“I”) expressed in external loop(s) of the capsid.

FIG. 3 illustrates a scheme for production of peptide multimers in VLPs in host cells, e.g., Pseudomonad host cells. The peptide insert is a multimer (a trimer is shown) of the desired target peptide(s), whose coding sequences (“i”) are inserted into the viral capsid coding sequence (“CP”) in constructing a recombinant viral capsid gene (“rCP”). Each of the target peptide coding sequences is bounded by coding sequences for cleavage sites (“*”) and the entire nucleic acid insert is labeled “I.”

FIG. 4 is an image of a SDS-PAGE gel (top) and western blot (bottom) showing expression of hydrophilicity unoptimized CCMV capsid protein (CP) with a BamHI restriction site at the position 129 expressed in Pseudomonas fluorescens separated into soluble and insoluble fractions 24 hours post-induction. The CCMV capsid proteins are indicated by arrows. Lane 1 is a size ladder (“M”), lane 2 is CP 0 hours post-induction (“0”), lane 3 is CP 24 hours post-induction (“24”).

FIG. 5 is an image of a SDS-PAGE gel showing expression of hydrophilicity-optimized CCMV capsid proteins in a Pseudomonas fluorescens bacterial system at 0, 6, 12, 18, and 24 hours post-induction in soluble and insoluble fractions. The soluble hydrophilicity-optimized CCMV capsid proteins are indicated by arrow and yielded >2 g/L. Lane 1 is a size ladder (“M”), lane 2 is a capsid protein (CP) standard for comparison.

FIG. 6 is an image of a western blot showing expression of hydrophilicity-optimized CCMV capsid proteins that have been purified from a Pseudomonas fluorescens bacterial system in a sucrose density gradient. Hydrophilicity-optimized CCMV VLPs were isolated 24 hours post-induction by PEG precipitation and fractionated on sucrose density gradient. The VLP fractions from the bottom band on the sucrose density gradient were positive for hydrophilicity-optimized CCMV capsid protein. Whole cell lysate, molecular weight ladder, PEG precipitated VLP fraction (sucrose gradient load), and top and bottom sucrose density fractions are indicated.

FIG. 7 is a transmission electron microscopy (“TEM”) image of soluble hydrophilicity-optimized CCMV VLPs purified from a Pseudomonas fluorescens bacterial system. The soluble hydrophilicity-optimized CCMV VLPs were isolated from Pseudomonas fluorescens using PEG precipitation and sucrose density fractionation.

FIG. 8 is an image of a SDS-PAGE gel showing expression of hydrophilicity unoptimized CCMV capsid proteins with a BamHI restriction site at the position 129 engineered to express a protective antigen of anthrax (“PA1”) expressed in Pseudomonas fluorescens. The capsid protein-PA1 fusion is indicated by arrow. CCMV capsid proteins were isolated at 0 and 24 hours post-induction by PEG precipitation and fractionated on sucrose density gradient. Lane 1 is a size ladder (“M”). The chimeric coat protein was mostly insoluble.

FIG. 9 is an image of a SDS-PAGE gel showing expression of hydrophilicity-optimized CCMV capsid proteins engineered to express a protective antigen of anthrax (“PA1”) expressed in Pseudomonas fluorescens. The capsid protein-PA1 fusion is indicated by arrow. Hydrophilicity-optimized CCMV capsid proteins were isolated 0, 6, 12, 18, and 24 hours post-induction. Lane 1 is a size ladder (“M”), lane 2 is a capsid protein (CP) standard for comparison. The chimeric coat protein was mostly soluble.

FIG. 10 illustrates the cloning of a hydrophilicity-optimized CCMV capsid fusion peptide into a Pseudomonas fluorescens plasmid using splicing by overlap extension (“SOE”)-based technology.

### DETAILED DESCRIPTION

It has been discovered that the overall amino acid composition of a capsid recombinant polypeptide is an important variable in the production of soluble VLPs. CP-fusion peptides with a high content of hydrophobic residues may have limited solubility in aqueous solution or may be completely insoluble. Increasing the hydrophilicity of the CP-fusion peptides may improve VLP solubility without adversely affecting the folding, assembly, or function of the VLP or peptide of insert. One particular strategy for increasing the hydrophilicity of a CP-fusion peptide includes increasing the hydrophilicity of either the capsid protein, peptide insert, or both across a focused area of amino acids.

The hydrophilicity of a protein or peptide that is encoded by a nucleic acid sequence in the construct can be determined by calculating the hydrophilic values of the amino acids contained across a particular area, wherein the calculations are based on a modified Roseman hydrophobicity scale (Table 2). The hydrophilicity of a VLP can be increased by the removal, mutagenesis, or addition of nucleic acid codons in the nucleic acid construct, wherein the codons encode amino acids. For example, based on the calculation of the hydrophilicity of a particular focused area, a nucleic acid construct can be altered in order to increase the hydrophilicity of that area. If an area has a low hydrophilicity value based on the modified Roseman hydrophobicity scale, then the codons in the area can be altered to increase the hydrophilicity of the area.

In one embodiment of the present invention, the hydrophilicity value of a focused area having low hydrophilicity can be increased by removing codons that have an undesirably low hydrophilicity value based on the modified Roseman hydrophobicity scale (Table 2). In an additional embodiment of the present invention, the hydrophilicity value of a focused area having low hydrophilicity can be increased by replacing a codon encoding an amino acid of low hydrophilicity with an amino acid having a higher hydrophilicity value based on the modified Roseman hydrophobicity scale (Table 2). Alternatively, the hydrophilicity of a focused area can be increased by adding one or more than one codons encoding amino acids with desirable hydrophilicity values according to the modified hydrophobicity scale (Table 1).

### I. Determination of Hydrophilicity

It has been found that VLP solubility is strongly influenced by the hydrophilic amino acid content of the CP-fusion peptides across particular areas. For example, CP-fusion peptides with focused areas of low hydrophilicity may have limited solubility. One focused area includes the area of insertion of a peptide of interest into a viral capsid fusion, including the restriction enzyme site. Increasing the hydrophilicity of a CP-fusion peptide over a focused area may provide for an increase in the yield of soluble VLPs produced in vivo.

The term “hydrophilicity-optimized,” as used herein, describes a nucleic acid construct comprising a capsid protein (CP) or CP-fusion peptide wherein the nucleic acid construct has been designed, engineered, or altered to increase the hydrophilicity of a focused area within the CP or CP-fusion peptide based on a modified Roseman hydrophobicity scale (Table 2).

A “hydrophilic” amino acid, as the term is used herein, refers to an amino acid with a modified Roseman hydrophobicity scale value of above 0.0.

The term “hydrophilicity %,” as used herein, refers to a particular amino acid sequence having the identified percentage of hydrophilic amino acids, wherein a hydrophilic amino acid has a modified Roseman hydrophobicity scale value of above 0.0. For example, a focused area of hydrophilicity encoding the amino acids Arg-Gly-Gly-Arg-Try-Trp could have a hydrophilicity of 66%.

The term “modified Roseman hydrophobicity scale,” as the term is used herein, refers to hydrophilicity values assigned to amino acids based on the modification of hydrophobicity data generated by Roseman (Hydrophilicity of polar amino acid side-chains is markedly reduced by flanking peptide bonds, J. Mol. Biol. 1999, 200:513-522), Kyte and Doolittle (A simple method for displaying the hydropathic character of a protein, J. Mol. Biol., 1982, 157:105-132), and Black and Mould (Development of hydrophobicity parameters to analyze proteins which bear post- or cotranslational modifications, Analytical Biochemistry, 1991, 193:72-82), and research done by Gunasekaran et al. (Beta-hairpins in proteins revisited: lessons for de novo design, Protein Eng. 1997, 10:1131-1141), the contents of each which are incorporated herein by reference. The Roseman, Kyte and Doolittle, and Black and Mould hydrophobicity scales are provided in Table 1.

TABLE 1
Non-modified hydrophobicity scales

| | | |
|---|---|---|
| | Kyte | Black and |
| Roseman | Doolittle | Mould |

| | | | | | | |
|---|---|---|---|---|---|---|
| 1 | Arg | −3.95 | Arg | −4.5 | Arg | −3.95 |
| 2 | Asp | −3.81 | Lys | −3.9 | Asp | −3.81 |
| 3 | Glu | −2.9 | Asn | −3.5 | Glu | −2.9 |
| 4 | Lys | −2.77 | Asp | −3.5 | Lys | −2.77 |
| 5 | Asn | −1.91 | Gln | −3.5 | Asn | −1.91 |
| 6 | Gln | −1.3 | Glu | −3.5 | Gln | −1.3 |
| 7 | Ser | −1.24 | His | −3.2 | Ser | −1.24 |
| 8 | His | −0.64 | Pro | −1.6 | His | −0.64 |
| 9 | Gly | 0 | Tyr | −1.3 | Gly | 0 |
| 10 | Cys | 0.25 | Trp | −0.9 | Cys | 0.25 |
| 11 | Ala | 0.39 | Ser | −0.8 | Ala | 0.39 |
| 12 | Met | 0.96 | Thr | −0.7 | Met | 0.96 |
| 13 | Pro | 0.99 | Gly | −0.4 | Pro | 0.99 |
| 14 | Thr | 1 | Ala | 1.8 | Thr | 1 |
| 15 | Val | 1.3 | Met | 1.9 | Val | 1.3 |
| 16 | Tyr | 1.47 | Cys | 2.5 | Tyr | 1.47 |
| 17 | Ile | 1.82 | Phe | 2.8 | Ile | 1.82 |
| 18 | Leu | 1.82 | Leu | 3.8 | Leu | 1.82 |
| 19 | Trp | 2.13 | Val | 4.2 | Trp | 2.13 |
| 20 | Phe | 2.27 | Ile | 4.5 | Phe | 2.27 |

Initially, the Roseman data was listed and scaled from 0 to 10 with 10 being the most hydrophilic, and 0 being the most hydrophobic. Because proline is commonly found in capsid protein loops (Ragone et al., Flexibility plot of proteins, Protein Eng. 1989, 7, 497-504), proline was placed just below cystine, which agrees with the data generated by Black and Mould. Additionally, due to the fact that threonine is commonly found in capsid protein loops (Ragone et al., Flexibility plot of proteins, Protein Eng. 1989, 7, 497-504), it was moved up between serine and glycine, which agrees with both the Kyte and Doolittle and the Black and Mould data. Then, methionine was chosen as the border for hydrophilicity based on the fact that small amino acids that are commonly found in flexible loops, such as alanine and proline, needed to be classified such that they would have higher preference than the hydrophobic amino acids. 2.4 was then subtracted from all of the numbers to make the value for methionine 0. The resultant modified Roseman hydrophobicity scale is provided in Table 2. The modified Roseman hydrophobicity scale is ordered with the most hydrophilic amino acids in a viral capsid protein setting having the highest positive value.

TABLE 2
Modified Roseman hydrophobicity scale

| | |
|---|---|
| Arg | 7.6 |
| Asp | 7.4 |
| Glu | 6.0 |
| Lys | 5.7 |
| Asn | 4.4 |
| Gln | 3.4 |
| Ser | 3.3 |
| Thr | 2.3 |
| Gly | 1.3 |
| His | 1.0 |
| Cys | 0.9 |
| Pro | 0.8 |
| Ala | 0.7 |
| Met | 0.0 |
| Val | −0.8 |
| Tyr | −1.1 |
| Ile | −1.6 |
| Leu | −1.6 |
| Trp | −2.1 |
| Phe | −2.4 |

### II. Nucleic Acid Constructs

The present invention provides hydrophilicity-optimized nucleic acid constructs encoding viral capsid proteins and CP-fusion peptides. In vivo expression of the encoded capsid protein or fusion peptide in a bacterial host system results in the enhanced production of soluble assembled VLPs. The hydrophilicity-optimized nucleic acid constructs of the present invention are designed by analyzing focused areas within the viral capsid protein or CP-fusion peptide, and adjusting areas of low hydrophilicity by modifying the area through addition, subtraction or mutagenesis of particular amino acids, including use of hydrophilicity-optimization rules based on the modified Roseman hydrophobicity scale.

In some embodiments of the present invention, the hydrophilicity value of a focused area within a nucleic acid construct having a low hydrophilicity value is increased by removing codons encoding amino acids that have a low hydrophilicity value, such as values of less than 0.0. In other embodiments of the present invention, the hydrophilicity value of a focused area within a nucleic acid construct having a low hydrophilicity value is increased by removing amino acids at position 63 and 129 insertion sites of the original CCMV coat protein construct by site directed mutagenesis or SOE.

In an additional embodiment of the present invention, the hydrophilicity value of an identified area having a low hydrophilicity value can be increased by replacing a codon encoding an amino acid of low hydrophilicity (less than 0.0) with an amino acid having a higher hydrophilicity value (greater than 0.0). Alternatively, the hydrophilicity of a focused area can be increased by adding one or more than one codons encoding amino acids with desirable hydrophilicity values (greater than 0.0).

In some embodiments of the present invention, a nucleic acid construct is provided wherein amino acids having a value of above 1.0 in the modified Roseman hydrophobicity scale are preferentially used to increase the hydrophobicity of a focused area. In other embodiments, amino acids having a value of above 1.0 in the modified Roseman hydrophobicity scale are added to a focused area in order to increase the hydrophilicity of the area. In additional embodiments, amino acids having a value of above 1.0 in the modified Roseman hydrophobicity scale are utilized to replace or substitute an amino acid having a value of less than 1.0.

### Hydrophilicity-Optimized Capsid Proteins

In some embodiments, the present invention provides isolated nucleic acid constructs encoding a hydrophilicity-optimized viral capsid protein. In a particular embodiment, the hydrophilicity-optimized capsid protein is derived from an icosahedral virus. In some embodiments, the optimized capsid protein is derived from icosahedral virus is CCMV. In other embodiments, the optimized capsid protein is derived from SEQ ID NO:1.

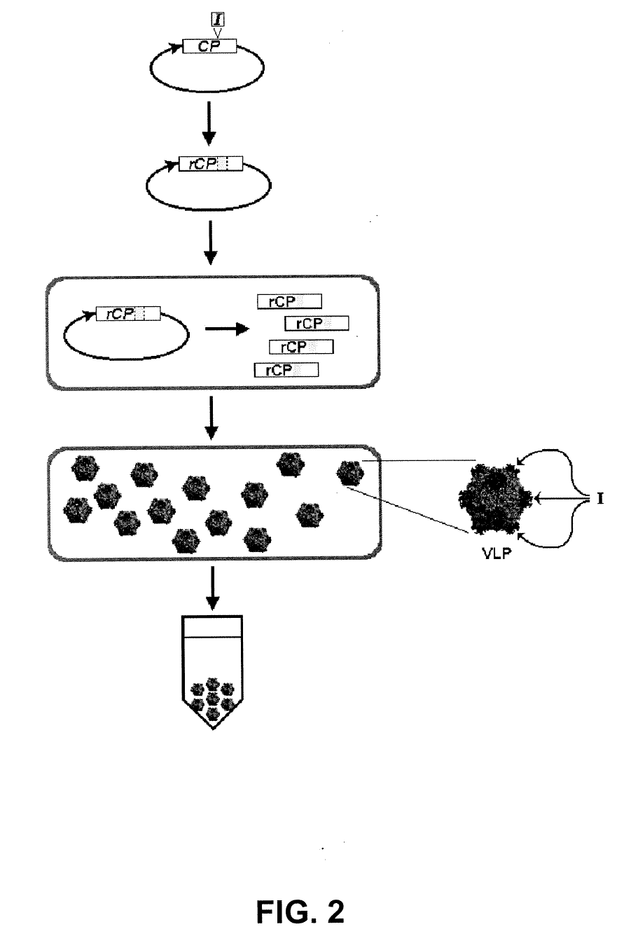

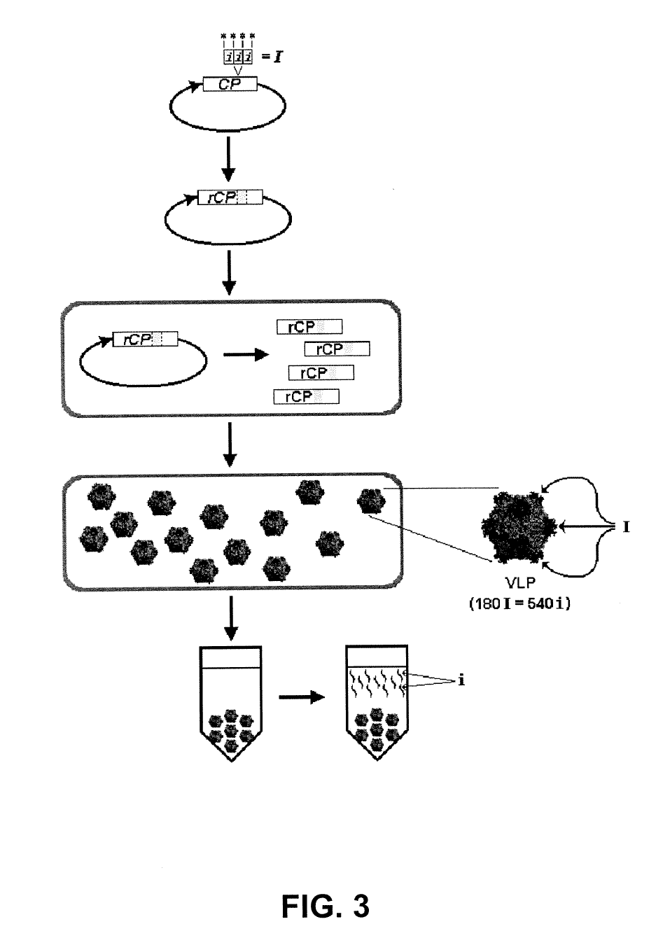

CCMV is a member of the bromovirus group of the Bromoviridae. Bromoviruses are 25-28 nm diameter icosahedral viruses with a four-component, positive sense, single-stranded RNA genome. RNA1 and RNA2 code for replicase enzymes. RNA3 codes for a protein involved in viral movement within plant hosts. RNA4 (a subgenomic RNA derived from RNA 3), i.e., sgRNA4, codes for the 20 kDa capsid protein (“CP”). Each CCMV particle contains up to about 180 copies of the CCMV CP. See FIGS. 2 and 3.

In one embodiment, the present invention provides nucleic acid constructs encoding a viral capsid protein, wherein the viral capsid protein contains an engineered restriction site encoding an area of hydrophilicity of at least 50%. The engineered restriction site provides an insertion site for a peptide of interest, allowing for the production of viral capsid protein-peptide fusion peptides (“CP-peptide fusions”) that can self-assemble into soluble VLPs. In one embodiment, the restriction site has an area of hydrophilicity of at least 50%, at least 55%, at least 60%, at least 65%, at least 70%, at least 75%, at least 80%, or at least 85%. In one embodiment, the engineered restriction site has an area of hydrophilicity of 100%.

In one embodiment, the engineered restriction site is comprised of nucleic acid codons encoding the amino acids Aspartic Acid, Glutamic Acid, Lysine, and Arginine (Asp-Glu-Lys-Arg). In one embodiment, the engineered restriction site does not contain codons encoding two or more consecutive hydrophobic amino acids selected from the group consisting of Alanine, Phenylalanine, Tryptophan, Tryptophan, Valine, Leucine, Methionine, or Proline. See Example 2. In one embodiment, the hydrophilicity-optimized restriction site is contained within a capsid protein derived from a CCMV capsid protein. In one embodiment, the hydrophilicity-optimized capsid protein comprises SEQ ID NO:2 with at least one amino acid inserted at a loop juncture.

In particular embodiments, criteria for choosing restriction sites for introducing a cloning site into CCMV capsid protein loops generally include: 1) the restriction sites should be absent from the ribosome binding site-CCMV CP open reading frame (ORF) cassette; 2) the restriction sites should be absent from the Pseudomonas fluorescens expression vector; and 3) the restriction site insertion should not result in introduction of amino acid Isoleucine followed by Leucine. In a general embodiment, the restriction site insertion should not be translatable into two or more consecutive hydrophobic amino acids, including Alanine, Phenylalanine, Tryptophan, Valine, Leucine, Isoleucine, Methionine, or Proline. Nonlimiting examples of restrictions sites chosen are: blunt-end cutters such as AfeI; 3′ overhang cutters such as BmtI and PvuI; and 5′ overhang cutters such as BglII, BsiWI, BspEI, BssSI, MluI, NheI and XbaI.

Amino acid sequence of CCMV CP containing focused areas of hydrophobicity (underlined): mstvgtgklt raqrraaark nkrntrvvqp vivepiasgq graikawtgy svskwtasca aaeawraaaa kvtsaitisl pnelssernk qlkvgrvllw lgllpsvsgt vkscvtetqt taaasfqval avadngilsk dvvaamypea fkgitleqlt adltiylyss aaltegdviv hlevehvrpt fddsftpvy (SEQ ID NO:1).

Amino acid sequence of original CCMV CP: mstvgtgklt raqrraaark nkrntrvvqp vivepiasgq gkaikawtgy svskwtasca aaeakvtsai tislpnelss ernkqlkvgr vllwlgllps vsgtvkscvt etqttaaasf qvalavadns kdvvaamype afkgitleql tadltiylys saaltegdvi vhlevehvrp tfddsftpvy (SEQ ID NO:2).

Nucleic acid sequence of hydrophilicity optimized CCMV CP for cloning into P. fluorescens expression vector (codon optimized for P. fluorescens). Contains SpeI restriction site, ribosome binding site, CP open reading frame (ORF), two stop codons, and XhoI restriction site: GGACTAGTAGGAGGTAACTTATGTCGACCGTGGGTACTGGGAAATTG ACTCGGGCACAACGTCGTGCTGCGGCCCGTAAGAATAAGCGCAAAACCCGCGTCGT CCAGCCTGTTATCGTCGAGCCAATCGCCTCGGGGCAAGGGAAAGCCATCAAGGCAT GGACCGGGTACTCGGTGAGCAAATGGACCGCGTCGTGCGCGGCAGCCGAGGCCAAA GTGACGAGCGCGATCACCATCAGCTTGCCTAACGAGCTGTCCAGCGAACGCAACAA GCAGCTCAAGGTCGGTCGTGTGCTGCTGTGGTTGGGCCTGCTCCCGAGCGTCTCCGG CACCGTGAAGTCGTGCGTGACGGAAACCCAGACGACTGCGGCCGCATCGTTCCAAG TGGCGCTCGCCGTGGCCGATAACAGCAAGGACGTGGTGGCCGCTATGTATCCTGAG GCCTTCAAGGGCATCACCCTGGAGCAGCTGACGGCCGACCTGACGATCTACCTGTAC TCCTCGGCCGCGTTGACCGAGGGCGATGTGATCGTGCACCTCGAAGTTGAACACGTG CGCCCGACTTTCGACGATTCCTTTACCCCGGTTTATTGATAATAGCTCGAGGC (SEQ ID NO:3).

Nucleic acid sequence of hydrophilicity optimized CCMV CP, codon optimized for expression in P. fluorescens: ATGTCGACCGTGGGTACTGGGAAATTGACTCGGGCAC AACGTCGTGCTGCGGCCCGTAAGAATAAGCGCAAAACCCGCGTCGTCCAGCCTGTT ATCGTCGAGCCAATCGCCTCGGGGCAAGGGAAAGCCATCAAGGCATGGACCGGGTA CTCGGTGAGCAAATGGACCGCGTCGTGCGCGGCAGCCGAGGCCAAAGTGACGAGCG CGATCACCATCAGCTTGCCTAACGAGCTGTCCAGCGAACGCAACAAGCAGCTCAAG GTCGGTCGTGTGCTGCTGTGGTTGGGCCTGCTCCCGAGCGTCTCCGGCACCGTGAAG TCGTGCGTGACGGAAACCCAGACGACTGCGGCCGCATCGTTCCAAGTGGCGCTCGC CGTGGCCGATAACAGCAAGGACGTGGTGGCCGCTATGTATCCTGAGGCCTTCAAGG GCATCACCCTGGAGCAGCTGACGGCCGACCTGACGATCTACCTGTACTCCTCGGCCG CGTTGACCGAGGGCGATGTGATCGTGCACCTCGAAGTTGAACACGTGCGCCCGACTT TCGACGATTCCTTTACCCCGGTTTATTGATAATAG (SEQ ID NO:4).

Variant of hydrophilicity and codon-optimized nucleic acid sequence of CCMV CP: ATGAGTACTGTTGGCACTGGTAAATTGACTCGGGCCCAGCGTCGTGCCGCCGCT CGCAAGAATAAGCGGAAGACCCGCGTGGTCCAACCTGTGATCGTGGAGCCCATCGC CTCCGGCCAGGGTAAAGCGATCAAAGCCTGGACGGGGTACAGTGTCAGCAAATGGA CGGCTTCGTGCGCTGCCGCGGAGGCCAAGGTCACGTCCGCTATCACCATTTCCCTGC CCAACGAGCTGAGCAGCGAGCGCAATAAGCAACTGAAGGTCGGCCGGGTCCTGCTG TGGCTGGGGTTGTTGCCTAGCGTGTCGGGCACCGTGAAGTCGTGCGTCACCGAAACC CAGACCACGGCAGCCGCTTCGTTCCAAGTGGCGCTGGCCGTCGCCGATAATTCCAAG GATGTCGTCGCGGCCATGTACCCGGAGGCTTTTAAGGGCATCACCCTGGAACAATTG ACCGCCGACCTGACTATCTACCTCTATTCGTCGGCTGCCTTGACTGAGGGCGACGTG ATCGTGCATTTGGAAGTCGAACACGTCCGTCCTACCTTTGACGACAGCTTTACCCCG GTGTACTGATAATAG (SEQ ID NO:5).

Codon-optimized nucleic acid sequence of Cowpea Chlorotic Mottle Virus (CCMV) capsid protein (CP) for expression in Pseudomonas fluorescens (coding region corresponds to SEQ ID NO:1): ATGTCGACCGTGGGTACTGGGAAATTGACTCGGGCAC AACGTCGTGCTGCGGCCCGTAAGAATAAGCGCAAAACCCGCGTCGTCCAGCCTGTT ATCGTCGAGCCAATCGCCTCGGGGCAAGGGAAAGCCATCAAGGCATGGACCGGGTA CTCGGTGAGCAAATGGACCGCGTCGTGCGCGGCAGCCGAGGCCAAAGTGACGAGCG CGATCACCATCAGCTTGCCTAACGAGCTGTCCAGCGAACGCAACAAGCAGCTCAAG GTCGGTCGTGTGCTGCTGTGGTTGGGCCTGCTCCCGAGCGTCTCCGGCACCGTGAAG TCGTGCGTGACGGAAACCCAGACGACTGCGGCCGCATCGTTCCAAGTGGCGCTCGC CGTGGCCGATAACAGCAAGGACGTGGTGGCCGCTATGTATCCTGAGGCCTTCAAGG GCATCACCCTGGAGCAGCTGACGGCCGACCTGACGATCTACCTGTACTCCTCGGCCG CGTTGACCGAGGGCGATGTGATCGTGCACCTCGAAGTTGAACACGTGCGCCCGACTT TCGACGATTCCTTTACCCCGGTTTATTGATAATAG.

Codon-optimized nucleic acid sequence of Cowpea Chlorotic Mottle Virus (CCMV) capsid protein (CP) for expression in Pseudomonas fluorescens: ATGAGTACTGTTG GCACTGGTAAATTGACTCGGGCCCAGCGTCGTGCCGCCGCTCGCAAGAATAAGCGG AAGACCCGCGTGGTCCAACCTGTGATCGTGGAGCCCATCGCCTCCGGCCAGGGTAA AGCGATCAAAGCCTGGACGGGGTACAGTGTCAGCAAATGGACGGCTTCGTGCGCTG CCGCGGAGGCCAAGGTCACGTCCGCTATCACCATTTCCCTGCCCAACGAGCTGAGCA GCGAGCGCAATAAGCAACTGAAGGTCGGCCGGGTCCTGCTGTGGCTGGGGTTGTTG CCTAGCGTGTCGGGCACCGTGAAGTCGTGCGTCACCGAAACCCAGACCACGGCAGC CGCTTCGTTCCAAGTGGCGCTGGCCGTCGCCGATAATTCCAAGGATGTCGTCGCGGC CATGTACCCGGAGGCTTTTAAGGGCATCACCCTGGAACAATTGACCGCCGACCTGAC TATCTACCTCTATTCGTCGGCTGCCTTGACTGAGGGCGACGTGATCGTGCATTTGGA AGTCGAACACGTCCGTCCTACCTTTGACGACAGCTTTACCCCGGTGTACTGATAATA G.

### Hydrophilicity-Optimized CP-Fusion Peptides

In an alternative embodiment of the present invention, hydrophilicity-optimized nucleic acid constructs are provided encoding CP-peptide fusions. CP-fusion peptide expression data suggests that a peptide inserted into a viral capsid protein should have a hydrophilicity of at least about 56%. In one embodiment of the present invention, a nucleic acid construct is provided wherein the encoded peptide insert is altered to increase the hydrophilicity of the peptide to attain a hydrophilicity of at least 56%. If the inserted peptide has a hydrophilicity of less than 56%, the hydrophilicity may be improved by adding extra amino acids, subtracting amino acids, or altering the nucleic acid sequence of existing amino acids in order to encode for amino acids with a more favorable hydrophilicity value.

In another embodiment of the invention, the inserted peptide can be modified by removing amino acids at position 63 and 129 insertion sites of the original CCMV coat protein construct by site directed mutagenesis or SOE.

In another embodiment, the inserted peptide can be hydrophilicity-optimized by adding one or more amino acids to the N- or C-terminus of the peptide having a modified Roseman hydrophobicity scale value of greater than 1.0. In an alternative embodiment, the inserted peptide can be optimized by removing one or more amino acids from the N- or C-terminus having a modified Roseman hydrophobicity value of less than 0.0. Alternatively, the inserted peptide can be optimized by replacing one or more amino acids with an amino acid having a greater value than said one or more amino acids on the modified Roseman hydrophobicity scale.

In a particular embodiment, the inserted peptide is optimized by adding amino acids with values above 1.0 to the N- or C-terminus of the peptide. Alternatively, the inserted peptide is optimized by adding amino acids with values above 0.0 to the N- or C-terminus of the peptide. In one embodiment, the inserted peptide is optimized by removing amino acids with a value of less than 0.0 from the N- or C-terminus of the peptide. In an alternative embodiment, the inserted peptide is optimized by replacing an amino acid with an amino acid having a higher value on the modified Roseman hydrophobicity scale.

In one embodiment, the peptide can be optimized by adding at least one amino acid selected from the group consisting of as Aspartic Acid, Glutamic Acid, Lysine, or Arginine to the N- or C-terminus in order to increase the hydrophilicity of the peptide.

Alternatively, the nucleic acid construct can be engineered to attain a hydrophilicity-optimized CP-peptide fusion. For example, the CP-peptide fusion can be optimized across an insert region by providing for a cloning strategy that allows for an increased focused area of hydrophilicity. In one embodiment, the hydrophilicity-optimized CP-protein of interest nucleic acid fusion is produced using restriction digest-based cloning methodology. The fusion can be, for example, of a sequence encoding a recombinant polypeptide and a hydrophilicity-optimized icosahedral capsid protein, wherein a recombinant polypeptide is fused with a hydrophilicity-optimized icosahedral capsid protein by restriction digest-based cloning methodology.

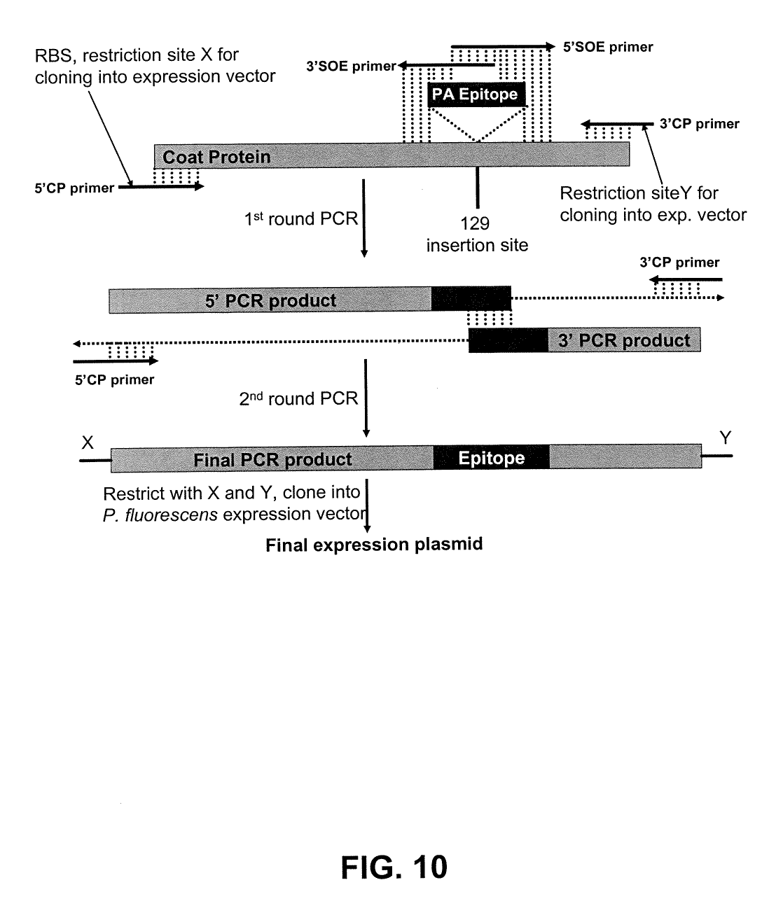

In some embodiments, the hydrophilicity-optimized CP-protein of interest nucleic acid fusion is produced using PCR-based technology. The fusion can be, for example, of a sequence encoding a recombinant polypeptide and a hydrophilicity-optimized icosahedral capsid protein wherein a recombinant polypeptide is fused with a hydrophilicity-optimized icosahedral capsid protein by PCR-based technology. In certain examples, the PCR-based technology is splicing by overlap extension (“SOE”), as illustrated in FIG. 10. The basic procedure of SOE is described by Horton et al. (1989) “Engineering hybrid genes without the use of restriction enzymes: Gene splicing by overlap extension,” Gene 77:61-68; and U.S. Pat. No. 5,023,171 to Ho et al., the contents of each of which is incorporated by reference. SOE is a method for joining two DNA molecules by first amplifying them by means of polymerase chain reactions (PCR) carried out on each molecule using oligonucleotide primers designed so that the ends of the resultant PCR products contain complementary sequences. When the two PCR products are mixed, denatured, and reannealed, the single-stranded DNA strands having the complementary sequences at their 3′ ends anneal and then act as primers for each other. Extension of the annealed area by DNA polymerase produces a double-stranded DNA molecule in which the original molecules are spliced together.

Additionally, the nucleic acid construct can be optimized following insertion of a peptide into the capsid protein by adding, removing, or altering amino acids in the restriction enzyme site. In one embodiment, the hydrophilicity of a CP-fusion peptide can be increased by removing a restriction enzyme site containing amino acids of undesirable hydrophilicity. In certain embodiments, the restriction site is altered or removed by mutagenesis after the fusion of the capsid protein and peptide insert. In certain embodiments, the restriction enzyme site is altered or removed by site-directed mutagenesis.

Amino acid sequence of CCMV CP with an insert of M2e-1 from influenza virus. M2e-1 amino acid sequence is shown in capital letters. Underlined residues were identified as focused areas of hydrophobicity. Percent (%) hydrophilicity is 65: mstvgtgklt raqrraaark nkrntrvvqp vivepiasgq graikawtgy svskwtasca aaeawraaaa kvtsaitisl pnelssernk qlkvgrvllw lgllpsvsgt vkscvtetqt taaasfqval avadn gilSLLTEVETPIRNEWGCRCNDSSDgil sk dvvaamypea fkgitleqlt adltiylyss aaltegdviv hlevehvrpt fddsftpvy (SEQ ID NO:6).

Amino acid sequence of hydrophilicity optimized CCMV CP with an insert of M2e-1 from influenza virus. M2e-1 amino acid sequence is shown in capital letters. Percent (%) hydrophilicity is 78%: mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn srSLLTEVETPIRNEWGCRCNDSSDsr sk dvvaamypea fkgitleqlt adltiylyss aaltegdviv hlevehvrpt fddsftpvy (SEQ ID NO:7).

Amino acid sequence of CCMV CP with an insert of PA1 from anthrax. The PA1 amino acid sequence is shown in capital letters. Underlined residues were identified as focused areas of hydrophobicity. Percent (%) hydrophilicity is 77: mstvgtgklt raqrraaark nkrntrvvqp vivepiasgq graikawtgy svskwtasca aaeawraaaa kvtsaitisl pnelssernk qlkvgrvllw lgllpsvsgt vkscvtetqt taaasfqval avadn gilSNSRKKRSTSAGPTVPDRDNDGIPD gil sk dvvaamypea fkgitleqlt adltiylyss aaltegdviv hlevehvrpt fddsftpvy (SEQ ID NO:8).

Amino acid sequence of hydrophilicity optimized CCMV CP with an insert of PA1 from anthrax. The PA1 amino acid sequence is shown in capital letters. Percent (%) hydrophilicity is 90: mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn srSNSRKKRSTSAGPTVPDRDNDGIPDsr sk dvvaamypea fkgitleqlt adltiylyss aaltegdviv hlevehvrpt fddsftpvy (SEQ ID NO:9).

Amino acid sequence of CCMV CP with an insert of PA4 from anthrax. The PA4 amino acid sequence is shown in capital letters. Underlined residues were identified as focused areas of hydrophobicity. Percent (%) hydrophilicity is 61: mstvgtgklt raqrraaark nkrntrvvqp vivepiasgq graikawtgy svskwtasca aaeawraaaa kvtsaitisl pnelssernk qlkvgrvllw lgllpsvsgt vkscvtetqt taaasfqval avadn gilRQDGKTFIDFKKYNDKLPLYISNPN gil sk dvvaamypea fkgitleqlt adltiylyss aaltegdviv hlevehvrpt fddsftpvy (SEQ ID NO:10).

Amino acid sequence of hydrophilicity optimized CCMV CP with an insert of PA4 from anthrax. The PA4 amino acid sequence is shown in capital letters. Percent (%) hydrophilicity is 72: mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn srRQDGKTFIDFKKYNDKLPLYISNPNsr sk dvvaamypea fkgitleqlt adltiylyss aaltegdviv hlevehvrpt fddsftpvy (SEQ ID NO:11).

Amino acid sequence of hydrophilicity optimized CCMV CP containing an M2e-1 insert produced using SOE (M2e-1 amino acid sequence shown in capital letters): mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn SLLTEVETPIRNEWGCRCNDSSDsk dvvaamypea fkgitleqlt adltiylyss aaltegdviv hlevehvrpt fddsftpvy (SEQ ID NO:12).

Amino acid sequence of hydrophilicity optimized CCMV CP containing a PA1 insert produced using SOE (PA1 amino acid sequence shown in capital letters): mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn SNSRKKRSTSAGPTVPDRDNDGIPD sk dvvaamypea fkgitleqlt adltiylyss aaltegdviv hlevehvrpt fddsftpvy (SEQ ID NO:13).

Amino acid sequence of hydrophilicity optimized CCMV CP containing a PA4 insert produced using SOE (PA4 amino acid sequence shown in capital letters): mstvgtgklt raqrraaark nkrntrvvqp vivepiasgq graikawtgy svskwtasca aaea kvtsaitisl pnelssernk qlkvgrvllw lgllpsvsgt vkscvtetqt taaasfqval avadn RQDGKTFIDFKKYNDKLPLYISNPN sk dvvaamypea fkgitleqlt adltiylyss aaltegdviv hlevehvrpt fddsftpvy (SEQ ID NO:14).

### Promoters

In one embodiment, the nucleic acid construct includes a promoter sequence operably attached to the nucleic acid sequence encoding the capsid protein-recombinant polypeptide fusion peptide. An operable attachment or linkage refers to any configuration in which the transcriptional and any translational regulatory elements are covalently attached to the described sequence so that by action of the host cell, the regulatory elements can direct the expression of the sequence of interest.

In a fermentation method, once expression of the target recombinant polypeptide is induced, it is ideal to have a high level of production in order to maximize efficiency of the expression system. The promoter initiates transcription and is generally positioned 10-100 nucleotides upstream of the ribosome binding site. Ideally, a promoter will be strong enough to allow for recombinant polypeptide accumulation of around 50% of the total cellular protein of the host cell, subject to tight regulation, and easily (and inexpensively) induced.

The promoters used in accordance with the present invention may be constitutive promoters or regulated promoters. Examples of commonly used inducible promoters and their subsequent inducers include lac (IPTG), lacUV5 (IPTG), tac (IPTG), trc (IPTG), P_{syn} (IPTG), trp (tryptophan starvation), araBAD (1-arabinose), lpp^{a} (IPTG), lpp-lac (IPTG), phoA (phosphate starvation), recA (nalidixic acid), proU (osmolarity), cst-1 (glucose starvation), tetA (tretracylin), cadA (pH), nar (anaerobic conditions), PL (thermal shift to 42° C.), cspA (thermal shift to 20° C.), T7 (thermal induction), T7-lac operator (IPTG), T3-lac operator (IPTG), T5-lac operator (IPTG), T4 gene32 (T4 infection), nprM-lac operator (IPTG), Pm (alkyl- or halo-benzoates), Pu (alkyl- or halo-toluenes), Psal (salicylates), and VHb (oxygen). See, for example, S. C. Makrides (1996) Microbiol. Rev. 60:512-538; G. Hannig and S. C. Makrides (1998) TIBTECH 16:54-60; R. C. Stevens (2000) Structures 8, R177-R185; J. Sanchez-Romero and V. De Lorenzo, Genetic Engineering of Nonpathogenic Pseudomonas strains as Biocatalysts for Industrial and Environmental Methodes, in Manual of Industrial Microbiology and Biotechnology (A. Demain and J. Davies, eds.) pp. 460-74 (1999) (ASM Press, Washington, D.C.); H. Schweizer, Vectors to express foreign genes and techniques to monitor gene expression for Pseudomonads, Current Opinion in Biotechnology, 12:439-445 (2001); and R. Slater and R. Williams, The Expression of Foreign DNA in Bacteria, in Molecular Biology and Biotechnology (J. Walker and R. Rapley, eds.) pp. 125-54 (2000) (The Royal Society of Chemistry, Cambridge, UK), the contents of each of which is incorporated by reference herein.

A promoter having the nucleotide sequence of a promoter native to the selected bacterial host cell can also be used to control expression of the transgene encoding the target polypeptide, e.g., a Pseudomonas anthranilate or benzoate operon promoter (Pant, Pben). Tandem promoters may also be used in which more than one promoter is covalently attached to another, whether the same or different in sequence, e.g., a Pant-Pben tandem promoter (interpromoter hybrid) or a Plac-Plac tandem promoter.

Regulated promoters utilize promoter regulatory proteins in order to control transcription of the gene of which the promoter is a part. Where a regulated promoter is used, a corresponding promoter regulatory protein can also be part of an expression system. Examples of promoter regulatory proteins include: activator proteins, e.g., E. coli catabolite activator protein, MalT protein; AraC family transcriptional activators; repressor proteins, e.g., E. coli LacI proteins; and dual-faction regulatory proteins, e.g., E. coli NagC protein. Many regulated-promoter/promoter-regulatory-protein pairs are known in the art.

Promoter regulatory proteins interact with an effector compound, i.e., a compound that reversibly or irreversibly associates with the regulatory protein, so as to enable the protein to either release or bind to at least one DNA transcription regulatory region of the gene that is under the control of the promoter, thereby permitting or blocking the action of a transcriptase enzyme in initiating transcription of the gene. Effector compounds are classified as either inducers or co-repressors, and these compounds include native effector compounds and gratuitous inducer compounds. Many regulated-promoter/promoter-regulatory-protein/effector-compound trios are known in the art. Although an effector compound can be used throughout the cell culture or fermentation, in a particular embodiment in which a regulated promoter is used, after growth of a desired quantity or density of host cell biomass, an appropriate effector compound is added to the culture in order to directly or indirectly result in expression of the desired target gene(s).

By way of example, where a lac family promoter is utilized, a lacI gene, or derivative thereof, such as a lacI^{Q} or lacI^{Q1} gene, can also be present in the system. The lacI gene, which is (normally) a constitutively expressed gene, encodes the Lac repressor protein (LacI protein) which binds to the lac operator of these promoters. Thus, where a lac family promoter is utilized, the lacI gene can also be included and expressed in the expression system. In the case of the lac promoter family members (e.g., the tac promoter) the effector compound is an inducer such as a gratuitous inducer such as IPTG (isopropyl-β-D-1-thiogalactopyranoside, also called “isopropylthiogalactoside”).

In a particular embodiment, a lac or tac family promoter is utilized in the present invention, including Plac, Ptac, Ptrc, PtacII, PlacUV5, lpp-PlacUV5, lpp-lac, nprM-lac, T7lac, T5lac, T3lac, and Pmac.

### Other Elements

Other regulatory elements can be included in an expression construct, including lacO sequences. Such elements include, but are not limited to, for example, transcriptional enhancer sequences, translational enhancer sequences, other promoters, activators, translational start and stop signals, transcription terminators, cistronic regulators, polycistronic regulators, tag sequences, such as nucleotide sequence “tags” and “tag” peptide coding sequences, which facilitates identification, separation, purification, or isolation of an expressed polypeptide, including His-tag, Flag-tag, T7-tag, S-tag, HSV-tag, B-tag, Strep-tag, polyarginine, polycysteine, polyphenylalanine, polyaspartic acid, (Ala-Trp-Trp-Pro)n, thioredoxin, beta-galactosidase, chloramphenicol acetyltransferase, cyclomaltodextrin gluconotransferase, CTP:CMP-3-deoxy-D-manno-octulosonate cytidyltransferase, trpE or trpLE, avidin, streptavidin, T7 gene 10, T4 gp55, Staphylococcal protein A, streptococcal protein G, GST, DHFR, CBP, MBP, galactose binding domain, Calmodulin binding domain, GFP, KSI, c-myc, ompT, ompA, pelB, NusA, ubiquitin, and hemosylin A.

In one embodiment, the nucleic acid construct further comprises a tag sequence adjacent to the coding sequence for the recombinant protein or peptide of interest, or a tag sequence linked to a coding sequence for a viral capsid protein. In one embodiment, this tag sequence allows for purification of the protein. The tag sequence can be an affinity tag, such as a hexa-histidine affinity tag. In another embodiment, the affinity tag can be a glutathione-S-transferase molecule. The tag can also be a fluorescent molecule, such as YFP or GFP, or analogs of such fluorescent proteins. The tag can also be a portion of an antibody molecule, or a known antigen or ligand for a known binding partner useful for purification.

The present invention can include, in addition to the capsid protein-recombinant polypeptide coding sequence, the following regulatory elements operably linked thereto: a promoter, a ribosome binding site (RBS), a transcription terminator, translational start and stop signals. Useful RBSs can be obtained from any of the species useful as host cells in expression systems according to the present invention. Many specific and a variety of consensus RBSs are known, e.g., those described in and referenced by D. Frishman et al., Starts of bacterial genes: estimating the reliability of computer predictions, Gene 234(2):257-65 (8 Jul. 1999); and B. E. Suzek et al., A probabilistic method for identifying start codons in bacterial genomes, Bioinformatics 17(12): 1123-30 (December 2001), the contents of which are incorporated by reference. In addition, either native or synthetic RBSs may be used, e.g., those described in: EP 0207459 (synthetic RBSs); O. Ikehata et al., Primary structure of nitrile hydratase deduced from the nucleotide sequence of a Rhodococcus species and its expression in Escherichia coli, Eur. J. Biochem. 181(3):563-70 (1989) (native RBS sequence of AAGGAAG), the contents of which are incorporated by reference. Further examples of methods, vectors, and translation and transcription elements, and other elements useful in the present invention are described in, e.g.: U.S. Pat. No. 5,055,294 to Gilroy and U.S. Pat. No. 5,128,130 to Gilroy et al.; U.S. Pat. No. 5,281,532 to Rammler et al.; U.S. Pat. Nos. 4,695,455 and 4,861,595 to Barnes et al.; U.S. Pat. No. 4,755,465 to Gray et al.; and U.S. Pat. No. 5,169,760 to Wilcox, the contents of which are incorporated by reference.

### Vectors

The nucleic acid constructs of the present invention can be contained in vectors capable of expression in a bacterial host cell. Generally, the recombinant expression vectors will include origins of replication and selectable markers permitting transformation of the host cell (e.g., the capsid protein-recombinant polypeptide fusion peptides of the present invention) and a promoter derived from a highly-expressed gene to direct transcription of a downstream structural sequence. The heterologous structural sequence is assembled in appropriate phase with translation initiation and termination sequences. Optionally, the heterologous sequence can encode a fusion polypeptide including an N-terminal identification peptide imparting desired characteristics, e.g., stabilization or simplified purification of expressed recombinant product.

Useful expression vectors for use in expressing capsid protein-recombinant polypeptide fusion peptides (for example, with P. fluorescens) can be constructed by inserting a structural DNA sequence encoding a desired target polypeptide fused with a capsid peptide together with suitable translation initiation and termination signals in operable reading phase with a functional promoter. The vector can comprise one or more phenotypic selectable markers and an origin of replication to ensure maintenance of the vector and, if desirable, to provide amplification within the host. Suitable hosts for transformation in accordance with the present disclosure include various species within the genera Pseudomonas, and, in particular, the host cell strain of Pseudomonas fluorescens.

Vectors are known in the art as useful for expressing recombinant proteins in host cells, and any of these may be modified and used for expressing the soluble fusion products in vivo according to the present invention. Such vectors include, e.g., plasmids, cosmids, and phage expression vectors. Examples of useful plasmid vectors that can be modified for use on the present invention include, but are not limited to, the expression plasmids pBBR1MCS, pDSK519, pKT240, pML122, pPS10, RK2, RK6, pRO1600, and RSF1010. Further examples can include pALTER-Ex1, pALTER-Ex2, pBAD/His, pBAD/Myc-His, pBAD/gIII, pCal-n, pCal-n-EK, pCal-c, pCal-Kc, pcDNA 2.1, pDUAL, pET-3a-c, pET 9a-d, pET-11a-d, pET-12a-c, pET-14b, pET15b, pET-16b, pET-17b, pET-19b, pET-20b(+), pET-21a-d(+), pET-22b(+), pET-23a-d(+), pET24a-d(+), pET-25b(+), pET-26b(+), pET-27b(+), pET28a-c(+), pET-29a-c(+), pET-30a-c(+), pET31b(+), pET-32a-c(+), pET-33b(+), pET-34b(+), pET35b(+), pET-36b(+), pET-37b(+), pET-38b(+), pET-39b(+), pET-40b(+), pET-41a-c(+), pET-42a-c(+), pET-43a-c(+), pETBlue-1, pETBlue-2, pETBlue-3, pGEMEX-1, pGEMEX-2, pGEX1λT, pGEX-2T, pGEX-2TK, pGEX-3X, pGEX-4T, pGEX-5X, pGEX-6P, pHAT10/11/12, pHAT20, pHAT-GFPuv, pKK223-3, pLEX, pMAL-c2X, pMAL-c2E, pMAL-c2g, pMAL-p2X, pMAL-p2E, pMAL-p2G, pProEX HT, pPROLar.A, pPROTet.E, pQE-9, pQE-16, pQE-30/31/32, pQE-40, pQE-50, pQE-70, pQE-80/81/82L, pQE-100, pRSET, and pSE280, pSE380, pSE420, pThioHis, pTrc99A, pTrcHis, pTrcHis2, pTriEx-1, pTriEx-2, pTrxFus. Other examples of such useful vectors include those described by, e.g.: N. Hayase, in Appl. Envir. Microbiol. 60(9):3336-42 (September 1994); A. A. Lushnikov et al., in Basic Life Sci. 30:657-62 (1985); S. Graupner and W. Wackernagel, in Biomolec. Eng. 17(1):11-16 (October 2000); H. P. Schweizer, in Curr. Opin. Biotech. 12(5):439-45 (October 2001); M. Bagdasarian and K. N. Timmis, in Curr. Topics Microbiol. Immunol. 96:47-67 (1982); T. Ishii et al., in FEMS Microbiol. Lett. 116(3):307-13 (Mar. 1, 1994); I. N. Olekhnovich and Y. K. Fomichev, in Gene 140(1):63-65 (Mar. 11, 1994); M. Tsuda and T. Nakazawa, in Gene 136(1-2):257-62 (Dec. 22, 1993); C. Nieto et al., in Gene 87(1):145-49 (Mar. 1, 1990); J. D. Jones and N. Gutterson, in Gene 61(3):299-306 (1987); M. Bagdasarian et al., in Gene 16(1-3):237-47 (December 1981); H. P. Schweizer et al., in Genet. Eng. (NY) 23:69-81 (2001); P. Mukhopadhyay et al., in J. Bact. 172(1):477-80 (January 1990); D. O. Wood et al., in J. Bact. 145(3):1448-51 (March 1981); and R. Holtwick et al., in Microbiology 147(Pt 2):337-44 (February 2001), the contents of each of which are incorporated by reference.

Further examples of expression vectors that can be useful in Pseudomonas host cells include those listed in the table below as derived from the indicated replicons.

NONLIMITING EXAMPLES OF USEFUL
EXPRESSION VECTORS

| Replicon | Vector(s) |
|---|---|
| pPS10 | pCN39, pCN51 |
| RSF1010 | pKT261-3 |
| | pMMB66EH |
| | pEB8 |
| | pPLGN1 |
| | pMYC1050 |
| RK2/RP1 | pRK415 |
| | pJB653 |
| pRO1600 | pUCP |
| | pBSP |

The expression plasmid, RSF1010, is described, e.g., by F. Heffron et al., in Proc. Nat'l Acad. Sci. USA 72(9):3623-27 (September 1975), and by K. Nagahari and K. Sakaguchi, in J. Bact. 133(3):1527-29 (March 1978), the contents of which are incorporated by reference. Plasmid RSF110 and derivatives thereof are particularly useful vectors in the present invention. Exemplary, useful derivatives of RSF1010, which are known in the art, include, e.g., pKT212, pKT214, pKT231 and related plasmids, and pMYC1050 and related plasmids (see, e.g., U.S. Pat. Nos. 5,527,883 and 5,840,554 to Thompson et al.), such as, e.g., pMYC1803. Plasmid pMYC1803 is derived from the RSF1010-based plasmid pTJS260 (see U.S. Pat. No. 5,169,760 to Wilcox), which carries a regulated tetracycline resistance marker and the replication and mobilization loci from the RSF1010 plasmid. Other exemplary useful vectors include those described in U.S. Pat. No. 4,680,264 to Puhler et al., the contents of which are incorporated by reference.

In other embodiments, an expression plasmid can be used as the expression vector. In another embodiment, RSF1010 or a derivative thereof can be used as the expression vector. In still another embodiment, pMYC1050 or a derivative thereof, or pMYC1803 or a derivative thereof, can be used as the expression vector.

The Champion™ pET expression system provides a high level of protein production. Expression is induced from the strong T7lac promoter. This system takes advantage of the high activity and specificity of the bacteriophage T7 RNA polymerase for high level transcription of the gene of interest. The lac operator located in the promoter region provides tighter regulation than traditional T7-based vectors, improving plasmid stability and cell viability (F. W. Studier and B. A. Moffatt (1986) J. Molecular Biology 189(1):113-30; Rosenberg, et al. (1987) Gene 56(1):125-35). The T7 expression system uses the T7 promoter and T7 RNA polymerase (T7 RNAP) for high-level transcription of the gene of interest. High-level expression can be achieved in T7 expression systems because the T7 RNAP is more methodive than native E. coli RNAP and is dedicated to the transcription of the gene of interest. Expression of the identified gene can be induced by providing a source of T7 RNAP in the host cell. This can be accomplished by using a BL21 E. coli host containing a chromosomal copy of the T7 RNAP gene. The T7 RNAP gene is under the control of the lacUV5 promoter, which can be induced by IPTG. T7 RNAP can be expressed upon induction and transcribes the gene of interest.

The pBAD expression system allows tightly controlled, titratable expression of recombinant protein through the presence of specific carbon sources such as glucose, glycerol and arabinose (Guzman, et al. (1995) J. Bacteriology 177(14):4121-30). The pBAD vectors are uniquely designed to give precise control over expression levels. Heterologous gene expression from the pBAD vectors is initiated at the araBAD promoter. The promoter is both positively and negatively regulated by the product of the araC gene. AraC is a transcriptional regulator that forms a complex with L-arabinose. In the absence of L-arabinose, the AraC dimer blocks transcription. For maximum transcriptional activation two events are required: (i) L-arabinose binds to AraC allowing transcription to begin. (ii.) The cAMP activator protein (CAP)-cAMP complex binds to the DNA and stimulates binding of AraC to the correct location of the promoter region.

The trc expression system allows high-level, regulated expression in E. coli from the trc promoter. The trc expression vectors have been optimized for expression of eukaryotic genes in E. coli. The trc promoter is a strong hybrid promoter derived from the tryptophane (trp) and lactose (lac) promoters. It is regulated by the lacO operator and the product of the lacIQ gene (J. Brosius (1984) Gene 27(2):161-72).

### III. Viral Capsid Proteins

The present invention utilizes capsid proteins derived from viruses. In some embodiments of the invention, the amino acid sequence of the capsid protein is selected from the capsid proteins of viruses classified as having icosahedral morphology. Icosahedral morphologies include icosahedral proper, isometric, quasi-isometric, and geminate or “twinned.” In other embodiments, the capsid protein amino acid sequence can be selected from the capsid proteins of entities that are icosahedral proper. In another embodiment, the capsid protein amino acid sequence can be selected from the capsid proteins of icosahedral viruses. In one particular embodiment, the capsid protein amino acid sequence can be selected from the capsid proteins of icosahedral plant viruses. However, in another embodiment, the viral capsid can be derived from an icosahedral virus not infectious to plants. For example, in one embodiment, the virus is a virus infectious to mammals.

Generally, viral capsids of icosahedral viruses are composed of numerous protein sub-units arranged in icosahedral (cubic) symmetry. Native icosahedral capsids can be built up, for example, with 3 subunits forming each triangular face of a capsid, resulting in 60 subunits forming a complete capsid. Representative of this small viral structure is, e.g., bacteriophage ØX174. Many icosahedral virus capsids contain more than 60 subunits. Many capsid proteins of icosahedral viruses contain an antiparallel, eight-stranded beta-barrel folding motif. The motif has a wedge-shaped block with four beta strands (designated BIDG) on one side and four (designated CHEF) on the other. There are also two conserved alpha-helices (designated A and B), one is between betaC and betaD, the other between betaE and betaF.

Enveloped viruses can exit an infected cell without its total destruction by extrusion (budding) of the particle through the membrane, during which the particle becomes coated in a lipid envelope derived from the cell membrane (See, e.g.: A. J. Cann (ed.) (2001) Principles of Molecular Virology (Academic Press); A. Granoff and R. G. Webster (eds.) (1999) Encyclopedia of Virology (Academic Press); D. L. D. Caspar (1980) Biophys. J. 32:103; D. L. D. Caspar and A. Klug (1962) Cold Spring Harbor Symp. Quant. Biol. 27: 1; J. Grimes et al. (1988) Nature 395:470; J. E. Johnson (1996) Proc. Nat'l Acad. Sci. USA 93:27; and J. Johnson and J. Speir (1997) J. Mol. Biol. 269:665).

### Viruses

Viral taxonomies recognize the following taxa of encapsidated-particle entities:
Group I Viruses, i.e., the dsDNA viruses;
Group II Viruses, i.e., the ssDNA viruses;
Group III Viruses, i.e., the dsRNA viruses;
Group IV Viruses, i.e., the ssRNA (+)-stranded viruses with no DNA stage;
Group V Viruses, i.e., the ssRNA (−)-stranded viruses;
Group VI Viruses, i.e., the RNA retroid viruses, which are ssRNA reverse transcribing viruses;
Group VII Viruses, i.e., the DNA retroid viruses, which are dsDNA reverse transcribing viruses;
Deltaviruses;
Viroids; and
Satellite phages and Satellite viruses, excluding Satellite nucleic acids and Prions.

The amino acid sequence of the capsid protein may be selected from the capsid proteins of any members of any of these taxa. Amino acid sequences for capsid proteins of the members of these taxa may be obtained from sources, including, but not limited to, e.g.: the on-line “Nucleotide” (Genbank), “Protein,” and “Structure” sections of the PubMed search facility offered by the NCBI at http://www.ncbi.nlm.nih.gov/entrez/query.fcgi.

In one embodiment, the capsid protein amino acid sequence will be selected from taxa members that are specific for at least one of the following hosts: fungi including yeasts, plants, protists including algae, invertebrate animals, vertebrate animals, and humans. In one embodiment, the capsid protein amino acid sequence will be selected from members of any one of the following taxa: Group I, Group II, Group III, Group IV, Group V, Group VII, Viroids, and Satellite Viruses. In one embodiment, the capsid protein amino acid sequence will be selected from members of any one of these seven taxa that are specific for at least one of the six above-described host types. In a more specific embodiment, the capsid protein amino acid sequence will be selected from members of any one of Group II, Group III, Group IV, Group VII, and Satellite Viruses; or from any one of Group II, Group IV, Group VII, and Satellite Viruses. In another embodiment, the viral capsid protein is selected from Group IV or Group VII.

The viral capsid protein sequence can be derived from a virus not tropic to the cell. In one embodiment, the cell does not include viral proteins from the particular selected virus other than the desired icosahedral protein. In one embodiment, the viral capsid can be derived from a virus with a tropism to a different family of organisms than the cell. In another embodiment, the viral capsid can be derived from a virus with a tropism to a different genus of organisms than the cell. In another embodiment, the viral capsid can be derived from a virus with a tropism to a different species of organisms than the cell. In a specific embodiment, the viral capsid can be selected from a virus of Group IV.

In one embodiment, the viral capsid is selected form an icosahedral virus. The icosahedral virus can be selected from a member of any of the Papillomaviridae, Totiviridae, Dicistroviridae, Hepadnaviridae, Togaviridiae, Polyomaviridiae, Nodaviridae, Tectiviridae, Leviviridae, Microviridae, Sipoviridae, Nodaviridae, Picornoviridae, Parvoviridae, Calciviridae, Tetraviridae, and Satellite viruses.

In a particular embodiment, the sequence can be selected from members of any one of the taxa that are specific for at least one plant host. In one embodiment, the icosahedral plant virus species will be a plant-infectious virus species that is, or is a member of, any of the Bunyaviridae, Reoviridae, Rhabdoviridae, Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In one embodiment, the icosahedral plant virus species is a plant-infectious virus species that is, or is a member of, any of the Luteoviridae, Nanoviridae, Partitiviridae, Sequiviridae, Tymoviridae, Ourmiavirus, Tobacco Necrosis Virus Satellite, Caulimoviridae, Geminiviridae, Comoviridae, Sobemovirus, Tombusviridae, or Bromoviridae taxa. In specific embodiments, the icosahedral plant virus species is a plant infectious virus species that is, or is a member of, any of the Caulimoviridae, Geminiviridae, Comoviridae, Sobemovirus, Tombusviridae, or Bromoviridae. In more particular embodiments, the icosahedral plant virus species will be a plant-infectious virus species that is, or is a member of, any of the Comoviridae, Sobemovirus, Tombusviridae, or Bromoviridae. In other embodiments, the icosahedral plant virus species will be a plant-infectious virus species that is a member of the Comoviridae or Bromoviridae family. In a particular embodiment, the viral capsid is derived from a Cowpea Mosaic Virus or a Cowpea Chlorotic Mottle Virus. In another embodiment, the viral capsid is derived from a species of the Bromoviridae taxa. In a specific embodiment, the capsid is derived from an Ilarvirus or an Alfamovirus. In a yet another embodiment, the capsid is derived from a Tobacco streak virus, an Alfalfa mosaic virus (“AMV”) (including AMV 1 or AMV 2).

### VLP

The icosahedral viral capsid protein of the invention is non-infective in the host cells described. In one embodiment, a soluble virus-like particle (“VLP”) or cage structure can be formed in the host cell during or after expression of the viral capsid protein. In one embodiment, the VLP or cage structure also includes the protein or peptide of interest, and in a particular embodiment, the protein or peptide of interest is expressed on the surface of the VLP. The expression system typically does not contain additional viral proteins that allow infectivity of the virus. In a typical embodiment, the expression system includes a host cell and a vector that codes for one or more viral capsid proteins and an operably linked protein or peptide of interest. The vector typically does not include additional viral assembly proteins.

In one embodiment, the VLP or cage structure is a multimeric assembly of capsid proteins, including from three to about 200 or more capsid proteins, as shown in FIGS. 2 and 3. In one embodiment, the VLP or cage structure includes at least 30, at least 50, at least 60, at least 90 or at least 120 capsid proteins. In another embodiment, each VLP or cage structure includes at least 150 capsid proteins, at least 160, at least 170, or at least 180 capsid proteins.

In one embodiment, the VLP is expressed as an icosahedral structure. In another embodiment, the VLP is expressed in the same geometry as that from which the native virus that the capsid sequence is derived. In a separate embodiment, however, the VLP does not have the identical geometry of the native virus. In certain embodiments, for example, the structure is produced in a particle formed of multiple capsids, but not forming a native-type VLP. For example, a cage structure of as few as 3 viral capsids can be formed. In separate embodiments, cage structures of about 6, 9, 12, 15, 18, 21, 24, 27, 30, 33, 36, 39, 42, 45, 48, 51, 54, 57, or 60 capsids can be formed.

In one embodiment, at least one of the capsid proteins includes at least one protein or peptide of interest. In one embodiment, the protein or peptide is expressed within at least one internal loop or in at least one external surface loop of the VLP.

In certain embodiments, the host cell can be modified to improve assembly of the VLP. The host cell can be modified, for example, to include chaperone proteins that promote the formation of VLPs from expressed viral capsids. In another embodiment, the host cell can be modified to include a repressor protein to more efficiently regulate the expression of the capsid protein to promote regulated formation of the VLPs.

The nucleic acid sequence encoding the viral capsid protein or proteins can also be additionally modified to alter the formation of VLPs (see, e.g., Brumfield, et al. (2004) J. Gen. Virol. 85:1049-1053). For example, three general classes of modification are most typically generated for modifying VLP expression and assembly. These modifications are designed to alter the interior, exterior, or the interface between adjacent subunits in the assembled protein cage. To accomplish this, mutagenic primers can be used to: (i) alter the interior surface charge of the viral nucleic acid binding region by replacing basic residues (e.g., K, R) in the N terminus with acidic glutamic acids (Douglas et al., 2002b); (ii) delete interior residues from the N terminus (in CCMV, usually residues 4-37); (iii) insert a cDNA encoding an 11 amino acid polypeptide cell-targeting sequence (Graf et al., 1987) into a surface exposed loop; and (iv) modify interactions between viral subunits by altering the metal binding sites (in CCMV, residues 81/148 mutant).

When the VLPs of the present invention may comprise a therapeutically active agent, they may also be used to treat disorders in a human or animal patient. Thus, the present invention can be used for treating a disease or disorder in a human or animal patient comprising administering to the patient an effective amount of VLPs of the present invention.

VLP immunogenic preparations or “cocktails” can also be used to administer a single vaccine that invokes a protective or therapeutically beneficial immune response against a multidude of infectious agents.

### IV. Recombinant Polypeptides

In one embodiment, the peptides or protein inserts operably linked to a viral capsid sequence contain at least two amino acids. In another embodiment, the proteins or peptides are at least three, at least four, at least five, or at least six amino acids in length. In a separate embodiment, the proteins or peptides are at least seven amino acids long. The proteins or peptides can also be at least eight, at least nine, at least ten, at least 11, 12, 13, 14, 15, 16, 17, 18, 19, 20, 30, 45, 50, 60, 65, 75, 85, 95, 96, 99 or more amino acids long. In one embodiment, the proteins or peptides encoded are at least 25 kD.

In one embodiment, the protein or peptide will contain from 2 to about 300 amino acids, or about 5 to about 250 amino acids, or about 5 to about 200 amino acids, or about 5 to about 150 amino acids, or about 5 to about 100 amino acids. In another embodiment, the protein or peptide contains from about 10 to about 140 amino acids, or from about 10 to about 120 amino acids, or from about 10 to about 100 amino acids.

In one embodiment, the peptides or proteins operably linked to a viral capsid sequence will contain about 500 amino acids. In another embodiment, the peptide will contain less than 500 amino acids. In yet another embodiment, the peptide can contain up to about 300 amino acids, or up to about 250, or up to about 200, or up to about 180, or up to about 160, or up to about 150, or up to about 140, or up to about 120, or up to about 110, or up to about 100, or up to about 90, or up to about 80, or up to about 70, or up to about 60, or up to about 50, or up to about 40 or up to about 30 amino acids.

In one embodiment, the recombinant polypeptide fused to the icosahedral capsid protein can be at least 7, at least 8, at least, 9, at least 10, at least 12, at least 15, at least 20, at least 25, at least 30, at least 35, at least 40, at least 45, at least 50, at least 55, at least 60, at least 65, at least 75, at least 85, at least 95, at least 99, or at least 100 amino acids.

In one embodiment of the present invention, the recombinant polypeptide contains at least one monomer of a desired target peptide. In an alternative embodiment, the recombinant polypeptide contains more than one monomer of a desired target peptide. In certain embodiments, the polypeptide is composed of at least two, at least 5, at least 10, at least 15 or at least 20 separate monomers that can be operably linked as a concatameric peptide to the capsid protein. In another embodiment, the individual monomers in the concatameric peptide can be linked by cleavable linker regions. In still another embodiment, the recombinant polypeptide can be inserted into at least one surface loop of the icosahedral virus-like particle. In one embodiment, at least one monomer can be inserted in a surface loop of the virus-like particle.

The proteins or peptides of interest that are fused to the viral capsid proteins can be a heterologous protein that is not derived from the virus and, optionally, that is not derived from the same species as the cell. The proteins or peptides of interest that are fused to the viral capsid proteins can be: functional peptides; structural peptides; antigenic peptides, toxic peptides, antimicrobial peptides, fragments thereof; precursors thereof; combinations of any of the foregoing; and/or concatamers of any of the foregoing. In one embodiment of the present invention, the recombinant polypeptide is a therapeutic peptide useful for human and animal treatments.

Functional peptides include, but are not limited to, e.g.: bio-active peptides (i.e., peptides that exert, elicit, or otherwise result in the initiation, enhancement, prolongation, attenuation, termination, or prevention of a biological function or activity in or of a biological entity, e.g., an organism, cell, culture, tissue, organ, or organelle); catalytic peptides; microstructure- and nanostructure-active peptides (i.e., peptides that form part of engineered micro- or nano-structures in which, or in conjunction with which, they perform an activity, e.g., motion, energy transduction); and stimulant peptides (e.g., peptide flavorings, colorants, odorants, pheromones, attractants, deterrents, and repellants).

Bio-active peptides include, but are not limited to, e.g.: immunoactive peptides (e.g., antigenic peptides, allergenic peptides, peptide immunoregulators, peptide immunomodulators); signaling and signal transduction peptides (e.g., peptide hormones, cytokines, and neurotransmitters; receptors; agonist and antagonist peptides; polypeptide targeting and secretion signal peptides); and bio-inhibitory peptides (e.g., toxic, biocidal, or biostatic peptides, such as peptide toxins and antimicrobial peptides).

Structural peptides include, but are not limited to, e.g.: peptide aptamers; folding peptides (e.g., peptides promoting or inducing formation or retention of a physical conformation in another molecule); adhesion-promoting peptides (e.g., adhesive peptides, cell-adhesion-promoting peptides); interfacial peptides (e.g., peptide surfactants and emulsifiers); microstructure and nanostructure-architectural peptides (i.e., structural peptides that form part of engineered micro- or nano-structures); and pre-activation peptides (e.g., leader peptides of pre-, pro-, and pre-pro-proteins and -peptides; inteins).

Catalytic Peptides include, e.g.: apo B RNA-editing cytidine deaminase peptides; catalytic peptides of glutaminyl-tRNA synthetases; catalytic peptides of aspartate transcarbamoylases; plant Type 1 ribosome-inactivating peptides; viral catalytic peptides such as, e.g., the foot-and-mouth disease virus [FMDV-2A] catalytic peptide; matrix metalloproteinase peptides; and catalytic metallo-oligopeptides.

The protein or peptide can also be a peptide s, haptens, or related peptides (e.g., antigenic viral peptides; virus related peptides, e.g., HIV-related peptides, hepatitis-related peptides; antibody idiotypic domains; cell surface peptides; antigenic human, animal, protist, plant, fungal, bacterial, and/or archaeal peptides; allergenic peptides and allergen desensitizing peptides).

The protein or peptide can also be: peptide immunoregulators or immunomodulators (e.g., interferons, interleukins, peptide immunodepressants and immunopotentiators); an antibody peptides (e.g., single chain antibodies; single chain antibody fragments and constructs, e.g., single chain Fv molecules; antibody light chain molecules, antibody heavy chain molecules, domain-deleted antibody light or heavy chain molecules; single chain antibody domains and molecules, e.g., a CH1, CH1-3, CH3, CH1-4, CH4, VHCH1, CL, CDR1, or FR1-CDR1-FR2 domain; paratopic peptides; microantibodies); another binding peptide (e.g., peptide aptamers, intracellular and cell surface receptor proteins, receptor fragments; anti-tumor necrosis factor peptides). The protein or peptide can also be an enzyme substrate peptide or an enzyme inhibitor peptide (e.g., caspase substrates and inhibitors, protein kinase substrates and inhibitors, fluorescence-resonance-energy transfer-peptide enzyme substrates).

The protein or peptide can also be: a cell surface receptor peptide ligand, agonist, and antagonist (e.g., caeruleins, dynorphins, orexins, pituitary adenylate cyclase activating peptides, tumor necrosis factor peptides; synthetic peptide ligands, agonists, and antagonists); a peptide hormone (e.g., endocrine, paracrine, and autocrine hormones, including, e.g.: amylins, angiotensins, bradykinins, calcitonins, cardioexcitatory neuropeptides, casomorphins, cholecystokinins, corticotropins and corticotropin-related peptides, differentiation factors, endorphins, endothelins, enkephalins, erythropoietins, exendins, follicle-stimulating hormones, galanins, gastrins, glucagons and glucagon-like peptides, gonadotropins, growth hormones and growth factors, insulins, kallidins, kinins, leptins, lipotropic hormones, luteinizing hormones, melanocyte stimulating hormones, melatonins, natriuretic peptides, neurokinins, neuromedins, nociceptins, osteocalcins, oxytocins (i.e., ocytocins), parathyroid hormones, pleiotrophins, prolactins, relaxins, secretins, serotonins, sleep-inducing peptides, somatomedins, thymopoietins, thyroid stimulating hormones, thyrotropins, urotensins, vasoactive intestinal peptides, vasopressins); a peptide cytokine, chemokine, virokine, and viroceptor hormone releasing and release-inhibiting peptide (e.g., corticotropin-releasing hormones, cortistatins, follicle-stimulating-hormone-releasing factors, gastric inhibitory peptides, gastrin releasing peptides, gonadotropin-releasing hormones, growth hormone releasing hormones, luteinizing hormone-releasing hormones, melanotropin-releasing hormones, melanotropin-release inhibiting factors; nocistatins, pancreastatins, prolactinreleasing peptides, prolactin release-inhibiting factors; somatostatins; thyrotropin releasing hormones); a peptide neurotransmitter or channel blocker (e.g., bombesins, neuropeptide Y, neurotensins, substance P) a peptide toxin, toxin precursor peptide, or toxin peptide portion. In certain embodiments, a peptide toxin contains no D-amino acids. Toxin precursor peptides can be those that contain no D-amino acids and/or that have not been converted by posttranslational modification into a native toxin structure, such as, e.g., by action of a D configuration inducing agent (e.g., a peptide isomerase(s) or epimeras(e) or racemase(s) or transaminase(s)) that is capable of introducing a D-configuration in an amino acid(s), and/or by action of a cyclizing agent (e.g., a peptide thioesterase, or a peptide ligase such as a trans-splicing protein or intein) that is capable of form a cyclic peptide structure.

Toxin peptide portions can be the linear or pre-cyclized oligo- and poly-peptide portions of peptide-containing toxins. Examples of peptide toxins include, e.g., agatoxins, amatoxins, charybdotoxins, chlorotoxins, conotoxins, dendrotoxins, insectotoxins, margatoxins, mast cell degranulating peptides, saporins, sarafotoxins; and bacterial exotoxins such as, e.g., anthrax toxins, botulism toxins, diphtheria toxins, and tetanus toxins.

The protein or peptide can also be: a metabolism- and digestion-related peptide (e.g., cholecystokinin-pancreozymin peptides, peptide yy, pancreatic peptides, motilins); a cell adhesion modulating or mediating peptide, extracellular matrix peptide (e.g., adhesins, selectins, laminins); a neuroprotectant or myelination-promoting peptide; an aggregation inhibitory peptide (e.g., cell or platelet aggregation inhibitor peptides, amyloid formation or deposition inhibitor peptides); a joining peptide (e.g., cardiovascular joining neuropeptides, iga joining peptides); or a miscellaneous peptide (e.g., agouti-related peptides, amyloid peptides, bone-related peptides, cell-permeable peptides, conantokins, contryphans, contulakins, myelin basic protein, and others).

In certain embodiments, the protein or peptide of interest can be exogenous to the selected viral capsid protein. Peptides may be either native or synthetic in sequence (and their coding sequences may be either native or synthetic nucleotide sequences). Thus, for example, native, modified native, and entirely artificial sequences of amino acids are encompassed. The sequences of the nucleic acid molecules encoding these amino acid sequences likewise may be native, modified native, or entirely artificial nucleic acid sequences, and may be the result of, e.g., one or more rational or random mutation and/or recombination and/or synthesis and/or selection method employed (i.e., applied by human agency) to obtain the nucleic acid molecules.

The coding sequence can be a native coding sequence for the target polypeptide, if available, but will more typically be a coding sequence that has been selected, improved, or optimized for use in the selected expression host cell: for example, by synthesizing the gene to reflect the codon use preference of a host species. In one embodiment of the invention, the host species is a P. fluorescens, and the codon preference of P. fluorescens is taken into account when designing both the signal sequence and the protein or peptide sequence.

### Antigenic Peptides

In one embodiment, an antigenic peptide is produced through expression with a viral capsid protein. The antigenic peptide can be selected from those that are antigenic peptides of human or animal pathogenic agents, including infectious agents, parasites, cancer cells, and other pathogenic agents. Such pathogenic agents also include the virulence factors and pathogenesis factors (e.g., exotoxins, endotoxins, et al.) of those agents. The pathogenic agents may exhibit any level of virulence, i.e., they may be, e.g., virulent, avirulent, pseudo-virulent, and semi-virulent. In one embodiment, the antigenic peptide can contain an epitopic amino acid sequence from the pathogenic agent(s). In another embodiment, the epitopic amino acid sequence can include at least a portion of a surface protein or peptide of at least one such agent. In one embodiment, the capsid protein-recombinant polypeptide VLPs can be used as a vaccine in a human or animal application.

More than one antigenic peptide may be selected, in which case, the resulting VLPs can present multiple different antigenic peptides. In a particular embodiment of a multiple antigenic peptide format, the various antigenic peptides can be selected from a plurality of or from the same pathogenic agent. In a particular embodiment of a multi-antigenic-peptide format, the various antigenic peptides selected can all be selected from a plurality of closely related pathogenic agents, for example, different strains, subspecies, biovars, pathovars, serovars, or genovars of the same species or different species of the same genus.

In one embodiment, the pathogenic agent(s) can belong to at least one of the following groups: Bacteria and Mycoplasma agents including, but not limited to, pathogenic: Bacillus spp., e.g., Bacillus anthracis; Bartonella spp., e.g., B. quintana; Brucella spp.; Burkholderia spp., e.g., B. pseudomallei; Campylobacter spp.; Clostridium spp., e.g., C. tetani, C. botulinum; Coxiella spp., e.g., C. burnetii; Edwardsiella spp., e.g., E. tarda; Enterobacter spp., e.g., E. cloacae; Enterococcus spp., e.g., E. faecalis, E. faecium; Escherichia spp., e.g., E. coli; Francisella spp., e.g., F. tularensis; Haemophilus spp., e.g., H. influenzae; Klebsiella spp., e.g., K. pneumoniae; Legionella spp.; Listeria spp., e.g., L. monocytogenes; Meningococci and Gonococci, e.g., Neisseria spp.; Moraxella spp.; Mycobacterium spp., e.g., M. leprae, M. tuberculosis; Pneumococci, e.g., Diplococcus pneumoniae; Pseudomonas spp., e.g., P. aeruginosa; Rickettsia spp., e.g., R. prowazekii, R. rickettsii, R. typhi; Salmonella spp., e.g., S. typhi; Staphylococcus spp., e.g., S. aureus; Streptococcus spp., including Group A Streptococci and hemolytic Streptococci, e.g., S. pneumoniae, S. pyogenes; Streptomyces spp.; Shigella spp.; Vibrio spp., e.g., V. cholerae; and Yersinia spp., e.g., Y. pestis, Y. enterocolitica. Fungus and Yeast agents include, but are not limited to, pathogenic: Alternaria spp.; Aspergillus spp.; Blastomyces spp., e.g., B. dermatiditis; Candida spp., e.g., C. albicans; Cladosporium spp.; Coccidiodes spp., e.g., C. immitis; Cryptococcus spp., e.g., C. neoformans; Histoplasma spp., e.g., H. capsulatum; and Sporothrix spp., e.g., S. schenckii.

In one embodiment, the pathogenic agent(s) can be from a protist agent that includes, but is not limited to, pathogenic: Amoebae, including Acanthamoeba spp., Amoeba spp., Naegleria spp., Entamoeba spp., e.g., E. histolytica; Cryptosporidium spp., e.g., C. parvum; Cyclospora spp.; Encephalitozoon spp., e.g., E. intestinalis; Enterocytozoon spp.; Giardia spp., e.g., G. lamblia; Isospora spp.; Microsporidium spp.; Plasmodium spp., e.g., P. falciparum, P. malariae, P. ovale, P. vivax; Toxoplasma spp., e.g., T. gondii; and Trypanosoma spp., e.g., T. brucei.

In one embodiment, the pathogenic agent(s) can be from a parasitic agent (e.g., helminthic parasites) including, but not limited to, pathogenic: Ascaris spp., e.g., A. lumbricoides; Dracunculus spp., e.g., D. medinensis; Onchocerca spp., e.g., O. volvulus; Schistosoma spp.; Trichinella spp., e.g., T. spiralis; and Trichuris spp., e.g., T. trichiura.

In another embodiment, the pathogenic agent(s) can be from a viral agent including, but not limited to, pathogenic: Adenoviruses; Arenaviruses, e.g., Lassa Fever viruses; Astroviruses; Bunyaviruses, e.g., Hantaviruses, Rift Valley Fever viruses; Coronaviruses, Deltaviruses; Cytomegaloviruses, Epstein-Barr viruses, Herpes viruses, Varicella viruses; Filoviruses, e.g., Ebola viruses, Marburg viruses; Flaviruses, e.g., Dengue viruses, West Nile Fever viruses, Yellow Fever viruses; Hepatitis viruses; Influenzaviruses; Lentiviruses, T-Cell Lymphotropic viruses, other leukemia viruses; Norwalk viruses; Papillomaviruses, other tumor viruses; Paramyxoviruses, e.g., Measles viruses, Mumps viruses, Parainfluenzaviruses, Pneumoviruses, Sendai viruses; Parvoviruses; Picornaviruses, e.g., Cardioviruses, Coxsackie viruses, Echoviruses, Poliomyelitis viruses, Rhinoviruses, Other Enteroviruses; Poxviruses, e.g., Variola viruses, Vaccinia viruses, Parapoxviruses; Reoviruses, e.g., Coltiviruses, Orbiviruses, Rotaviruses; Rhabdoviruses, e.g., Lyssaviruses, Vesicular Stomatitis viruses; and Togaviruses, e.g., Rubella viruses, Sindbis viruses, Western Encephalitis viruses.

In one particular embodiment, the antigenic peptide can be selected from the group consisting of a Canine parvovirus peptide, anthracis protective antigenic peptide, and an Eastern Equine Encephalitis virus antigenic peptide. In a particular embodiment, the antigenic peptide is the anthracis protective antigen peptide with any one of the amino acid sequence of SEQ ID NOS:16, 17, 18 or 19. In still another particular embodiment, the antigenic peptide is an Eastern equine Encephalitis virus antigenic peptide with the amino acid sequence of one of SEQ ID NOS:20 or 21.

Nucleic Acid Sequences Encoding Various Peptide Inserts

| | | SEQ |
|---|---|---|
| Sequence | Name | ID |
| SNSRKKRSTSAGPTVPDRDNDGIPD | Amino acid sequence | 16 |
| | of Bacillus anthracis | |
| | protective antigen 1 | |
| | (“PA1”) peptide | |
| SPEARHPLVAAYPIVHVDMENIILS | Amino acid sequence | 17 |
| | of B. anthracis | |
| | protective antigen 2 | |
| | (“PA2”) peptide | |
| RIIFNGKDLNLVERRIAAVNPSDPL | Amino acid sequence | 18 |
| | of B. anthracis | |
| | protective antigen 3 | |
| | (“PA3”) peptide | |
| RQDGKTFIDFKKYNDKLPLYISNPN | Amino acid sequence | 19 |
| | of B. anthracis | |
| | protective antigen 4 | |
| | (“PA4”) peptide | |
| DLDTHFTQYKLARPYIADCPNCGHS | Amino acid sequence | 20 |
| | of Eastern equine | |
| | encephalomyelitis | |
| | virus antigen 1 | |
| | (“EEE1”) peptide | |
| GRLPRGEGDTFKGKLHVPFVPVKAK | Amino acid sequence | 21 |
| | of Eastern equine | |
| | encephalomyelitis | |
| | virus protective | |
| | antigen 2 (“EEE2”) | |
| | peptide | |

### Host-Cell Toxic Peptide

In another particular embodiment, the recombinant polypeptide is a peptide that is toxic to the host cell when in free monomeric form. In a more particular embodiment, the toxic peptide is an antimicrobial peptide.

In certain embodiments, the protein or peptide of interest expressed in conjunction with a viral capsid protein can be a host cell toxic peptide. In certain embodiments, this protein will be an antimicrobial protein or peptide. A host cell toxic peptide indicates a bio-inhibitory peptide that is biostatic, biocidal, or toxic to the host cell in which it is expressed, or to other cells in the cell culture or organism of which the host cell is a member, or to cells of the organism or species providing the host cells. In one embodiment, the host-cell-toxic peptide can be a bioinhibitory peptide that is biostatic, biocidal, or toxic to the host cell in which it is expressed. Some examples of host-cell-toxic peptides include, but are not limited to: peptide toxins; anti-microbial peptides; and other antibiotic peptides. Anti-Microbial Peptides include, e.g.: anti-bacterial peptides such as, e.g., magainins, betadefensins, some alpha-defensins; cathelicidins; histatins; anti-fungal peptides; antiprotozoal peptides; synthetic AMPs; peptide antibiotics or the linear or pre-cyclized oligo- or poly-peptide portions thereof; other antibiotic peptides (e.g., anthelmintic peptides, hemolytic peptides, tumoricidal peptides); and anti-viral peptides (e.g., some alpha-defensins; virucidal peptides; peptides that inhibit viral infection). In one particular embodiment, the antimicrobial peptide (“AMP”) is the D2A21 peptide with the amino acid sequence of SEQ ID NO:22.

Name: Amino acid sequence of D2A21 trimer antimicrobial protective antigen (“PA”) peptide: FAKKFAKKFKKFAKKFAKFAFAFGDPFAKKFAKKFKKFAKKFAKFAFA FGDPFAKKFAKKFKKFAKKFAKFAFAFG (SEQ ID NO:22).

In one embodiment, the recombinant polypeptide is hydrophilicity optimized and, for example, comprises a hydrophobic amino acid content below 50% and, in certain instances, at least one charged (i.e., polar) amino acid residue for every five amino acids. In another embodiment, the recombinant polypeptide comprises a hydrophobic amino acid content below 40% and at least one charged amino acid residue for every four amino acids.

The invention also provides Pseudomonad organisms with a nucleic acid construct encoding a fusion peptide of a hydrophilicity-optimized CP-recombinant polypeptide. In one specific embodiment of the present invention, the Pseudomonad cell is Pseudomonas fluorescens. In one embodiment, the cell produces soluble assembled VLPs in vivo. In one embodiment, the protein or peptide of interest can be a therapeutic peptide useful for human and animal treatments.

The invention also provides a method for producing a recombinant polypeptide cell by providing a nucleic acid encoding a fusion peptide of a recombinant polypeptide and a hydrophilicity-optimized icosahedral capsid protein; expressing the nucleic acid wherein the expression in the cell provides for in vivo production of soluble assembled VLPs and isolating the VLPs. In one embodiment, the cell is a Pseuodmonad and in certain embodiments is a P. fluorescens.

### V. Recombinant Pseudomonad Cells

The present invention further provides bacterial host cells comprising a hydrophilicity-optimized nucleic acid construct encoding a viral capsid protein or CP-peptide fusion. In one embodiment, the bacterial host cell is selected from the group consisting of a Pseudomonad cell. In one embodiment, the cell is a Pseudomonas fluorescens. In another embodiment, the cell is E. coli. The cells can be utilized in a method for producing recombinant polypeptides.

### Cells for Use in Expressing the VLP

Typical bacterial cells are described, for example, in “Biological Diversity: Bacteria and Archaeans,” a chapter of the On-Line Biology Book, provided by Dr. M. J. Farabee of the Estrella Mountain Community College, Arizona, USA at URL: http://www.emc.maricopa.edu/faculty/farabee/BIOBK/BioBookDiversity_{—}2.html. In one embodiment, the host cell can be a member of any species of eubacteria. The host can be a member any one of the taxa: Acidobacteria, Actinobacteira, Aquificae, Bacteroidetes, Chlorobi, Chlamydiae, Choroflexi, Chrysiogenetes, Cyanobacteria, Deferribacteres, Deinococcus, Dictyoglomi, Fibrobacteres, Firmicutes, Fusobacteria, Gemmatimonadetes, Lentisphaerae, Nitrospirae, Planctomycetes, Proteobacteria, Spirochaetes, Thermodesulfobacteria, Thermomicrobia, Thermotogae, Thermus (Thermales), or Verrucomicrobia. In an embodiment of a eubacterial host cell, the cell can be a member of any species of eubacteria, excluding Cyanobacteria.

The bacterial host can also be a member of any species of Proteobacteria. A proteobacterial host cell can be a member of any one of the taxa Alphaproteobacteria, Betaproteobacteria, Gammaproteobacteria, Deltaproteobacteria, or Epsilonproteobacteria. In addition, the host can be a member of any one of the taxa Alphaproteobacteria, Betaproteobacteria, or Gammaproteobacteria, and a member of any species of Gammaproteobacteria.

In one embodiment of a Gamma Proteobacterial host, the host can be a member of any one of the taxa Aeromonadales, Alteromonadales, Enterobacteriales, Pseudomonadales, or Xanthomonadales; or a member of any species of the Enterobacteriales or Pseudomonadales. In one embodiment, the host cell can be of the order Enterobacteriales, the host cell will be a member of the family Enterobacteriaceae, or a member of any one of the genera Erwinia, Escherichia, or Serratia; or a member of the genus Escherichia. In one embodiment of a host cell of the order Pseudomonadales, the host cell will be a member of the family Pseudomonadaceae, even of the genus Pseudomonas. Gamma Proteobacterial hosts include members of the species Escherichia coli and members of the species Pseudomonas fluorescens.

Other Pseudomonas organisms may also be used. Pseudomonads and closely related species include Gram(−) Proteobacteria Subgroup 1, which include the group of Proteobacteria belonging to the families and/or genera described as “Gram-Negative Aerobic Rods and Cocci” by R. E. Buchanan and N. E. Gibbons (eds.), Bergey's Manual of Determinative Bacteriology, pp. 217-289 (8th ed., 1974) (The Williams and Wilkins Co., Baltimore, Md., USA) (hereinafter “Bergey (1974)”), the contents of which are incorporated by reference herein.

“Gram(−) Proteobacteria Subgroup 1” also includes Proteobacteria that would be classified in this heading according to the criteria used in the classification. The heading also includes groups that were previously classified in this section but are no longer, such as the genera Acidovorax, Brevundimonas, Burkholderia, Hydrogenophaga, Oceanimonas, Ralstonia, and Stenotrophomonas, the genus Sphingomonas (and the genus Blastomonas, derived therefrom), which was created by regrouping organisms belonging to (and previously called species of) the genus Xanthomonas, the genus Acidomonas, which was created by regrouping organisms belonging to the genus Acetobacter as defined in Bergey (1974). In addition hosts can include cells from the genus Pseudomonas, Pseudomonas enalia (ATCC 14393), Pseudomonas nigrifaciens (ATCC 19375), and Pseudomonas putrefaciens (ATCC 8071), which have been reclassified respectively as Alteromonas haloplanktis, Alteromonas nigrifaciens, and Alteromonas putrefaciens. Similarly, e.g., Pseudomonas acidovorans (ATCC 15668) and Pseudomonas testosteroni (ATCC 11996) have since been reclassified as Comamonas acidovorans and Comamonas testosteroni, respectively; and Pseudomonas nigrifaciens (ATCC 19375) and Pseudomonas piscicida (ATCC 15057) have been reclassified respectively as Pseudoalteromonas nigrifaciens and Pseudoalteromonas piscicida.

“Gram(−) Proteobacteria Subgroup 1” also includes Proteobacteria classified as belonging to any of the families: Pseudomonadaceae, Azotobacteraceae (now often called by the synonym, the “Azotobacter group” of Pseudomonadaceae), Rhizobiaceae, and Methylomonadaceae (now often called by the synonym, “Methylococcaceae”). Consequently, in addition to those genera described herein, further Proteobacterial genera falling within “Gram(−) Proteobacteria Subgroup 1” include: 1) Azotobacter group bacteria of the genus Azorhizophilus; 2) Pseudomonadaceae family bacteria of the genera Cellvibrio, Oligella, and Teredinibacter; 3) Rhizobiaceae family bacteria of the genera Chelatobacter, Ensifer, Liberibacter (also called “Candidatus Liberibacter”), and Sinorhizobium; and 4) Methylococcaceae family bacteria of the genera Methylobacter, Methylocaldum, Methylomicrobium, Methylosarcina, and Methylosphaera.

In another embodiment, the host cell can be selected from “Gram(−) Proteobacteria Subgroup 2.” “Gram(−) Proteobacteria Subgroup 2” is defined as the group of Proteobacteria of the following genera (with the total numbers of catalog-listed, publicly-available, deposited strains thereof indicated in parenthesis, all deposited at ATCC, except as otherwise indicated): Acidomonas (2); Acetobacter (93); Gluconobacter (37); Brevundimonas (23); Beijerinckia (13); Derxia (2); Brucella (4); Agrobacterium (79); Chelatobacter (2); Ensifer (3); Rhizobium (144); Sinorhizobium (24); Blastomonas (1); Sphingomonas (27); Alcaligenes (88); Bordetella (43); Burkholderia (73); Ralstonia (33); Acidovorax (20); Hydrogenophaga (9); Zoogloea (9); Methylobacter (2); Methylocaldum (1 at NCIMB); Methylococcus (2); Methylomicrobium (2); Methylomonas (9); Methylosarcina (1); Methylosphaera; Azomonas (9); Azorhizophilus (5); Azotobacter (64); Cellvibrio (3); Oligella (5); Pseudomonas (1139); Francisella (4); Xanthomonas (229); Stenotrophomonas (50); and Oceanimonas (4).

Exemplary host cell species of “Gram(−) Proteobacteria Subgroup 2” include, but are not limited to the following bacteria (with the ATCC or other deposit numbers of exemplary strain(s) thereof shown in parenthesis): Acidomonas methanolica (ATCC 43581); Acetobacter aceti (ATCC 15973); Gluconobacter oxydans (ATCC 19357); Brevundimonas diminuta (ATCC 11568); Beijerinckia indica (ATCC 9039 and ATCC 19361); Derxia gummosa (ATCC 15994); Brucella melitensis (ATCC 23456), Brucella abortus (ATCC 23448); Agrobacterium tumefaciens (ATCC 23308), Agrobacterium radiobacter (ATCC 19358), Agrobacterium rhizogenes (ATCC 11325); Chelatobacter heintzii (ATCC 29600); Ensifer adhaerens (ATCC 33212); Rhizobium leguminosarum (ATCC 10004); Sinorhizobium fredii (ATCC 35423); Blastomonas natatoria (ATCC 35951); Sphingomonas paucimobilis (ATCC 29837); Alcaligenes faecalis (ATCC 8750); Bordetella pertussis (ATCC 9797); Burkholderia cepacia (ATCC 25416); Ralstonia pickettii (ATCC 27511); Acidovorax facilis (ATCC 11228); Hydrogenophaga flava (ATCC 33667); Zoogloea ramigera (ATCC 19544); Methylobacter luteus (ATCC 49878); Methylocaldum gracile (NCIMB 11912); Methylococcus capsulatus (ATCC 19069); Methylomicrobium agile (ATCC 35068); Methylomonas methanica (ATCC 35067); Methylosarcina fibrata (ATCC 700909); Methylosphaera hansonii (ACAM 549); Azomonas agilis (ATCC 7494); Azorhizophilus paspali (ATCC 23833); Azotobacter chroococcum (ATCC 9043); Cellvibrio mixtuis (UQM 2601); Oligella urethralis (ATCC 17960); Pseudomonas aeruginosa (ATCC 10145), Pseudomonas fluorescens (ATCC 35858); Francisella tularensis (ATCC 6223); Stenotrophomonas maltophilia (ATCC 13637); Xanthomonas campestris (ATCC 33913); and Oceanimonas doudoroffii (ATCC 27123).

In another embodiment, the host cell can be selected from “Gram(−) Proteobacteria Subgroup 3.” “Gram(−) Proteobacteria Subgroup 3” is defined as the group of Proteobacteria of the following genera: Brevundimonas; Agrobacterium; Rhizobium; Sinorhizobium; Blastomonas; Sphingomonas; Alcaligenes; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In another embodiment, the host cell can be selected from “Gram(−) Proteobacteria Subgroup 4.” “Gram(−) Proteobacteria Subgroup 4” is defined as the group of Proteobacteria of the following genera: Brevundimonas; Blastomonas; Sphingomonas; Burkholderia; Ralstonia; Acidovorax; Hydrogenophaga; Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

In one embodiment, the host cell is selected from “Gram(−) Proteobacteria Subgroup 5.” “Gram(−) Proteobacteria Subgroup 5” is defined as the group of Proteobacteria of the following genera: Methylobacter; Methylocaldum; Methylococcus; Methylomicrobium; Methylomonas; Methylosarcina; Methylosphaera; Azomonas; Azorhizophilus; Azotobacter; Cellvibrio; Oligella; Pseudomonas; Teredinibacter; Francisella; Stenotrophomonas; Xanthomonas; and Oceanimonas.

The host cell can be selected from “Gram(−) Proteobacteria Subgroup 6.” “Gram(−) Proteobacteria Subgroup 6” is defined as the group of Proteobacteria of the following genera: Brevundimonas; Blastomonas; Sphingomonas; Burkholderia; Ralstonia; Acidovorax; Hydrogenophaga; Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram(−) Proteobacteria Subgroup 7.” “Gram(−) Proteobacteria Subgroup 7” is defined as the group of Proteobacteria of the following genera: Azomonas; Azorhizophilus; Azotobacter; Cellvibrio; Oligella; Pseudomonas; Teredinibacter; Stenotrophomonas; Xanthomonas; and Oceanimonas.

The host cell can be selected from “Gram(−) Proteobacteria Subgroup 8.” “Gram(−) Proteobacteria Subgroup 8” is defined as the group of Proteobacteria of the following genera: Brevundimonas; Blastomonas; Sphingomonas; Burkholderia; Ralstonia; Acidovorax; Hydrogenophaga; Pseudomonas; Stenotrophomonas; Xanthomonas; and Oceanimonas.

The host cell can be selected from “Gram(−) Proteobacteria Subgroup 9.” “Gram(−) Proteobacteria Subgroup 9” is defined as the group of Proteobacteria of the following genera: Brevundimonas; Burkholderia; Ralstonia; Acidovorax; Hydrogenophaga; Pseudomonas; Stenotrophomonas; and Oceanimonas.

The host cell can be selected from “Gram(−) Proteobacteria Subgroup 10.” “Gram(−) Proteobacteria Subgroup 10” is defined as the group of Proteobacteria of the following genera: Burkholderia; Ralstonia; Pseudomonas; Stenotrophomonas; and Xanthomonas.

The host cell can be selected from “Gram(−) Proteobacteria Subgroup 11.” “Gram(−) Proteobacteria Subgroup 11” is defined as the group of Proteobacteria of the genera: Pseudomonas; Stenotrophomonas; and Xanthomonas.

The host cell can be selected from “Gram(−) Proteobacteria Subgroup 12.” “Gram(−) Proteobacteria Subgroup 12” is defined as the group of Proteobacteria of the following genera: Burkholderia; Ralstonia; Pseudomonas. The host cell can be selected from “Gram(−) Proteobacteria Subgroup 13.” “Gram(−) Proteobacteria Subgroup 13” is defined as the group of Proteobacteria of the following genera: Burkholderia; Ralstonia; Pseudomonas; and Xanthomonas. The host cell can be selected from “Gram(−) Proteobacteria Subgroup 14.” “Gram(−) Proteobacteria Subgroup 14” is defined as the group of Proteobacteria of the following genera: Pseudomonas and Xanthomonas. The host cell can be selected from “Gram(−) Proteobacteria Subgroup 15.” “Gram(−) Proteobacteria Subgroup 15” is defined as the group of Proteobacteria of the genus Pseudomonas.

The host cell can be selected from “Gram(−) Proteobacteria Subgroup 16.” “Gram(−) Proteobacteria Subgroup 16” is defined as the group of Proteobacteria of the following Pseudomonas species (with the ATCC or other deposit numbers of exemplary strain(s) shown in parenthesis): Pseudomonas abietaniphila (ATCC 700689); Pseudomonas aeruginosa (ATCC 10145); Pseudomonas alcaligenes (ATCC 14909); Pseudomonas anguilliseptica (ATCC 33660); Pseudomonas citronellolis (ATCC 13674); Pseudomonas flavescens (ATCC 51555); Pseudomonas mendocina (ATCC 25411); Pseudomonas nitroreducens (ATCC 33634); Pseudomonas oleovorans (ATCC 8062); Pseudomonas pseudoalcaligenes (ATCC 17440); Pseudomonas resinovorans (ATCC 14235); Pseudomonas straminea (ATCC 33636); Pseudomonas agarici (ATCC 25941); Pseudomonas alcaliphila; Pseudomonas alginovora; Pseudomonas andersonii; Pseudomonas asplenii (ATCC 23835); Pseudomonas azelaica (ATCC 27162); Pseudomonas beijerinckii (ATCC 19372); Pseudomonas borealis; Pseudomonas boreopolis (ATCC 33662); Pseudomonas brassicacearum; Pseudomonas butanovora (ATCC 43655); Pseudomonas cellulosa (ATCC 55703); Pseudomonas aurantiaca (ATCC 33663); Pseudomonas chlororaphis (ATCC 9446, ATCC 13985, ATCC 17418, ATCC 17461); Pseudomonas fragi (ATCC 4973); Pseudomonas lundensis (ATCC 49968); Pseudomonas taetrolens (ATCC 4683); Pseudomonas cissicola (ATCC 33616); Pseudomonas coronafaciens; Pseudomonas diterpeniphila; Pseudomonas elongata (ATCC 10144); Pseudomonas flectens (ATCC 12775); Pseudomonas azotoformans; Pseudomonas brenneri; Pseudomonas cedrella; Pseudomonas corrugata (ATCC 29736); Pseudomonas extremorientalis; Pseudomonas fluorescens (ATCC 35858); Pseudomonas gessardii; Pseudomonas libanensis; Pseudomonas mandelii (ATCC 700871); Pseudomonas marginalis (ATCC 10844); Pseudomonas migulae; Pseudomonas mucidolens (ATCC 4685); Pseudomonas orientalis; Pseudomonas rhodesiae; Pseudomonas synxantha (ATCC 9890); Pseudomonas tolaasii (ATCC 33618); Pseudomonas veronii (ATCC 700474); Pseudomonas frederiksbergensis; Pseudomonas geniculata (ATCC 19374); Pseudomonas gingeri; Pseudomonas graminis; Pseudomonas grimontii; Pseudomonas halodenitrificans; Pseudomonas halophila; Pseudomonas hibiscicola (ATCC 19867); Pseudomonas huttiensis (ATCC 14670); Pseudomonas hydrogenovora; Pseudomonas jessenii (ATCC 700870); Pseudomonas kilonensis; Pseudomonas lanceolata (ATCC 14669); Pseudomonas lini; Pseudomonas marginata (ATCC 25417); Pseudomonas mephitica (ATCC 33665); Pseudomonas denitrificans (ATCC 19244); Pseudomonas pertucinogena (ATCC 190); Pseudomonas pictorum (ATCC 23328); Pseudomonas psychrophila; Pseudomonas fulva (ATCC 31418); Pseudomonas monteilii (ATCC 700476); Pseudomonas mosselii; Pseudomonas oryzihabitans (ATCC 43272); Pseudomonas plecoglossicida (ATCC 700383); Pseudomonas putida (ATCC 12633); Pseudomonas reactans; Pseudomonas spinosa (ATCC 14606); Pseudomonas balearica; Pseudomonas luteola (ATCC 43273); Pseudomonas stutzeri (ATCC 17588); Pseudomonas amygdali (ATCC 33614); Pseudomonas avellanae (ATCC 700331); Pseudomonas caricapapayae (ATCC 33615); Pseudomonas cichorii (ATCC 10857); Pseudomonas ficuserectae (ATCC 35104); Pseudomonas fuscovaginae; Pseudomonas meliae (ATCC 33050); Pseudomonas syringae (ATCC 19310); Pseudomonas viridiflava (ATCC 13223); Pseudomonas thermocarboxydovorans (ATCC 35961); Pseudomonas thermotolerans; Pseudomonas thivervalensis; Pseudomonas vancouverensis (ATCC 700688); Pseudomonas wisconsinensis; and Pseudomonas xiamenensis.

The host cell can be selected from “Gram(−) Proteobacteria Subgroup 17.” “Gram(−) Proteobacteria Subgroup 17” is defined as the group of Proteobacteria known in the art as the “fluorescent Pseudomonads” including those belonging, e.g., to the following Pseudomonas species: Pseudomonas azotoformans; Pseudomonas brenneri; Pseudomonas cedrella; Pseudomonas corrugata; Pseudomonas extremorientalis; Pseudomonas fluorescens; Pseudomonas gessardii; Pseudomonas libanensis; Pseudomonas mandelii; Pseudomonas marginalis; Pseudomonas migulae; Pseudomonas mucidolens; Pseudomonas orientalis; Pseudomonas rhodesiae; Pseudomonas synxantha; Pseudomonas tolaasii; and Pseudomonas veronii.

In this embodiment, the host cell can be selected from “Gram(−) Proteobacteria Subgroup 18.” “Gram(−) Proteobacteria Subgroup 18” is defined as the group of all subspecies, varieties, strains, and other sub-special units of the species Pseudomonas fluorescens, including those belonging, e.g., to the following (with the ATCC or other deposit numbers of exemplary strain(s) shown in parenthesis): Pseudomonas fluorescens biotype A, also called biovar 1 or biovar I (ATCC 13525); Pseudomonas fluorescens biotype B, also called biovar 2 or biovar II (ATCC 17816); Pseudomonas fluorescens biotype C, also called biovar 3 or biovar III (ATCC 17400); Pseudomonas fluorescens biotype F, also called biovar 4 or biovar IV (ATCC 12983); Pseudomonas fluorescens biotype G, also called biovar 5 or biovar V (ATCC 17518); Pseudomonas fluorescens biovar VI; Pseudomonas fluorescens Pf0-1; Pseudomonas fluorescens Pf-5 (ATCC BAA-477); Pseudomonas fluorescens SBW25; and Pseudomonas fluorescens subsp. cellulosa (NCIMB 10462).

The host cell can be selected from “Gram(−) Proteobacteria Subgroup 19.” “Gram(−) Proteobacteria Subgroup 19” is defined as the group of all strains of Pseudomonas fluorescens biotype A. A particular strain of this biotype is P. fluorescens strain MB 101 (see U.S. Pat. No. 5,169,760 to Wilcox), and derivatives thereof. An example of a derivative thereof is P. fluorescens strain MB214, constructed by inserting into the MB101 chromosomal asd (aspartate dehydrogenase gene) locus, a native E. coli PlacI-lacI-lacZYA construct (i.e., in which PlacZ was deleted).

Additional P. fluorescens strains that can be used in the present invention include Pseudomonas fluorescens Migula and Pseudomonas fluorescens Loitokitok, having the following ATCC designations: [NCIB 8286]; NRRL B-1244; NCIB 8865 strain CO1; NCIB 8866 strain CO2; 1291 [ATCC 17458; IFO 15837; NCIB 8917; LA; NRRL B-1864; pyrrolidine; PW2 [ICMP 3966; NCPPB 967; NRRL B-899]; 13475; NCTC 10038; NRRL B-1603 [6; IFO 15840]; 52-1C; CCEB 488-A [BU 140]; CCEB 553 [IEM 15/47]; IAM 1008 [AHH-27]; IAM 1055 [AHH-23]; 1 [IFO 15842]; 12 [ATCC 25323; NIH 11; den Dooren de Jong 216]; 18 [IFO 15833; WRRL P-7]; 93 [TR-10]; 108 [52-22; IFO 15832]; 143 [IFO 15836; PL]; 149 [2-40-40; IFO 15838]; 182 [IFO 3081; PJ 73]; 184 [IFO 15830]; 185 [W2 L-1]; 186 [IFO 15829; PJ 79]; 187 [NCPPB 263]; 188 [NCPPB 316]; 189 [PJ227; 1208]; 191 [IFO 15834; PJ 236; 22/1]; 194 [Klinge R-60; PJ 253]; 196 [PJ 288]; 197 [PJ 290]; 198 [PJ 302]; 201 [PJ 368]; 202 [PJ 372]; 203 [PJ 376]; 204 [IFO 15835; PJ 682]; 205 [PJ 686]; 206 [PJ 692]; 207 [PJ 693]; 208 [PJ 722]; 212 [PJ 832]; 215 [PJ 849]; 216 [PJ 885]; 267 [B-9]; 271 [B-1612]; 401 [C71A; IFO 15831; PJ 187]; NRRL B-3178 [4; IFO 15841]; KY 8521; 3081; 30-21; [IFO 3081]; N; PYR; PW; D946-B83 [BU 2183; FERM-P 3328]; P-2563 [FERM-P 2894; IFO 13658]; IAM-1126 [43F]; M-1; A506 [A5-06]; A505 [A5-05-1]; A526 [A5-26]; B69; 72; NRRL B-4290; PMW6 [NCIB 11615]; SC 12936; Al [IFO 15839]; F 1847 [CDC-EB]; F 1848 [CDC 93]; NCIB 10586; P17; F-12; AmMS 257; PRA25; 6133D02; 6519E01; N1; SC15208; BNL-WVC; NCTC 2583 [NCIB 8194]; H13; 1013 [ATCC 11251; CCEB 295]; IFO 3903; 1062; or Pf-5.

### VI. In Vivo Expression of Soluble Assembled Virus-Like Particles in Pseudomonads

In one aspect, the present invention provides a method for the in vivo production of soluble assembled recombinant virus-like particles in a host cell including:
(a) providing a host cell;
(b) providing an isolated nucleic acid encoding a hydrophilicity-optimized CP-peptide fusion
(c) expressing the isolated nucleic acid in the host cell, wherein the expression in the cell provides for in vivo assembly of the hydrophilicity-optimized CP-fusion peptide into soluble virus-like particles; and
(d) isolating the virus-like particles.

In one embodiment, the method can further include: e) cleaving the fusion peptide product to separate the recombinant polypeptide from the capsid protein. In one embodiment of the present invention, the host cell is a Pseudomonad cell and in a particular embodiment, is Pseudomonas fluorescens. In one embodiment, the isolated virus-like particle can be administered to a human or animal in a vaccine strategy. A cleavable linkage sequence can be included between the viral capsid protein and the recombinant polypeptide. Examples of agents that can cleave such sequences include, but are not limited to chemical reagents such as acids (HCl, formic acid), CNBr, hydroxylamine (for asparagine-glycine), 2-Nitro-5-thiocyanobenzoate, O-Iodosobenzoate, and enzymatic agents, such as endopeptidases, endoproteases, trypsin, clostripain, and Staphylococcal protease.

In another embodiment, a second nucleic acid, which is designed to express a different protein or peptide, such as a chaperone protein, can be expressed concomitantly with the nucleic acid encoding the soluble fusion peptide.

The bacterial host cells, capsid proteins, and recombinant polypeptides useful for the present invention are discussed above.

In some embodiments, the method produces at least 0.1 g/L protein in the form of soluble VLPs. In another embodiment, the method produces 0.1 to 10 g/L protein in the form of soluble VLPs. In subembodiments, the method produces at least about 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, or more than 2.0 such as 2.1, 2.2, 2.3, 2.4, 2.5 or more g/L protein in the form of soluble VLPs. In one embodiment, the total recombinant protein produced is at least 1.0 or at least 2.0 g/L. In some embodiments, the amount of VLP protein produced is at least about 5%, about 10%, about 15%, about 20%, about 25%, about 30%, about 40%, about 50%, about 60%, about 70%, about 80%, about 90%, about 95% or more of total recombinant protein produced.

In subembodiments, the total soluble VLPs produced can be at least about 2.0, 3.0, 4.0, 5.0, 6.0, 7.0, 8.0, 9.0, 10.0, 15.0, 20.0 or 50.0 g/L. In some embodiments, the amount of VLPs produced as soluble assembled VLPs is at least about 5%, about 10%, about 15%, about 20%, about 25%, or more of total recombinant protein produced.

In other embodiments, the method produces recombinant protein as 5, 10, 15, 20, 25, 30, 40 or 50, 55, 60, 65, 70, or 75% of total cell protein (tcp). “Percent total cell protein” is the amount of protein or peptide in the host cell as a percentage of aggregate cellular protein. The determination of the percent total cell protein is well known in the art.

In a particular embodiment, the host cell can have a recombinant peptide, polypeptide, protein, or fragment thereof expression level of at least 1% tcp and a cell density of at least 40 g/L, when grown (i.e., within a temperature range of about 4° C. to about 55° C., inclusive) in a mineral salts medium. In a particular embodiment, the expression system will have a recombinant protein of peptide expression level of at least 5% tcp and a cell density of at least 40 g/L, when grown (i.e., within a temperature range of about 4° C. to about 55° C., inclusive) in a mineral salts medium at a fermentation scale of at least 10 Liters.

### Expression Levels

The method of the invention optimally leads to increased production of soluble VLPs in a host cell. The increased production alternatively can be an increased level of active protein or peptide per gram of protein produced, or per gram of host protein. The increased production can also be an increased level of recoverable protein or peptide, produced per gram of recombinant or per gram of host cell protein. The increased production can also be any combination of increased total level and increased active or soluble level of protein.

The improved expression of recombinant protein can be through expression of the protein encapsulated in VLPs. In certain embodiments, at least 60, at least 70, at least 80, at least 90, at least 100, at least 110, at least 120, at least 130, at least 140, at least 150, at least 160, at least 170, or at least 180 copies of a protein or peptide of interest can be expressed in each VLP. The VLPs can be produced and recovered from the cytoplasm, periplasm or extracellular medium of the host cell.

The protein or peptide or viral capsid sequence can also include one or more targeting sequences or sequences to assist purification. These can be an affinity tagged and can also be targeting sequences directing the assembly of capsid proteins into a VLP.

### Cell Growth

Transformation of the bacterial host cells, including Pseudomonas host cells, with the vector(s) may be performed using any transformation methodology known in the art, and the bacterial host cells may be transformed as intact cells or as protoplasts (i.e., including cytoplasts). Exemplary transformation methodologies include poration methodologies, e.g., electroporation, protoplast fusion, bacterial conjugation, and divalent cation treatment, e.g., calcium chloride treatment or CaCl/Mg2+ treatment, or other well known methods in the art.

As used herein, the term “fermentation” includes both embodiments in which literal fermentation is employed and embodiments in which other, non-fermentative culture modes are employed. Fermentation may be performed at any scale. In one embodiment, the fermentation medium may be selected from among rich media, minimal media, and mineral salts media. In another embodiment, either a minimal medium or a mineral salts medium is selected.

Mineral salts media consists of mineral salts and a carbon source such as, e.g., glucose, sucrose, or glycerol. Examples of mineral salts media include, e.g., M9 medium, Pseudomonas medium (ATCC 179), Davis and Mingioli medium (see, B. D. Davis and E. S. Mingioli (1950) in J. Bact. 60:17-28). The mineral salts used to make mineral salts media include those selected from among, e.g., potassium phosphates, ammonium sulfate or chloride, magnesium sulfate or chloride, and trace minerals such as calcium chloride, borate, and sulfates of iron, copper, manganese, and zinc. No organic nitrogen source, such as peptone, tryptone, amino acids, or a yeast extract, is included in a mineral salts medium. Instead, an inorganic nitrogen source is used and this may be selected from among, e.g., ammonium salts, aqueous ammonia, and gaseous ammonia. One mineral salts medium will contain glucose as the carbon source. In comparison to mineral salts media, minimal media can also contain mineral salts and a carbon source, but can be supplemented with, e.g., low levels of amino acids, vitamins, peptones, or other ingredients, though these are added at very minimal levels.

The high cell density culture can start as a batch method, which is followed by a two-phase fed-batch cultivation. After unlimited growth in the batch part, growth can be controlled at a reduced specific growth rate over a period of three doubling times in which the biomass concentration can increase several fold. Further details of such cultivation procedures is described by D. Riesenberg, V. Schulz, W. A. Knorre, H. D. Pohl, D. Korz, E. A. Sanders, A. Ross, and W. D. Deckwer (1991) “High cell density cultivation of Escherichia coli at controlled specific growth rate,” J. Biotechnol. 20(1) 17-27, the contents of which are incorporated by reference herein.

The expression system according to the present invention can be cultured in any fermentation format. For example, batch, fed-batch, semi-continuous, and continuous fermentation modes may be employed herein.

The expression systems according to the present invention are useful for transgene expression at any scale (i.e., volume) of fermentation. Thus, e.g., microliter-scale, centiliter scale, and deciliter scale fermentation volumes may be used; and 1 Liter scale and larger fermentation volumes can be used. In one embodiment, the fermentation volume can be at or above 1 Liter. In another embodiment, the fermentation volume can be at or above 5 Liters, 10 Liters, 15 Liters, 20 Liters, 25 Liters, 50 Liters, 75 Liters, 100 Liters, 200 Liters, 500 Liters, 1,000 Liters, 2,000 Liters, 5,000 Liters, 10,000 Liters or 50,000 Liters.

In the present invention, growth, culturing, and/or fermentation of the transformed host cells is performed within a temperature range permitting survival of the host cells, such as at a temperature within the range of about 4° C. to about 55° C., inclusive. Thus, e.g., the terms “growth” (and “grow,” “growing”), “culturing” (and “culture”), and “fermentation” (and “ferment,” “fermenting”), as used herein in regard to the host cells of the present invention, inherently means “growth,” “culturing,” and “fermentation,” within a temperature range of about 4° C. to about 55° C., inclusive. In addition, “growth” is used to indicate both biological states of active cell division and/or enlargement, as well as biological states in which a non-dividing and/or non-enlarging cell is being metabolically sustained, the latter use of the term “growth” being synonymous with the term “maintenance.”

### Isolation of Protein or Peptide of Interest

In certain embodiments, the invention provides a method for improving the recovery of proteins or peptides of interest by protection of the protein or peptide during expression through linkage and co-expression with a soluble viral capsid protein. In certain embodiments, the soluble viral capsid fusion form soluble VLPs in vivo, which can be readily separated from the cell lysate.

To release recombinant proteins from the periplasm, any suitable method known in the art can be employed. Examples of such methods include osmotic shock, hen egg white (HEW)-lysozyme/ethylenediamine tetraacetic acid (EDTA) treatment, and combined HEW-lysozyme/osmotic shock treatment. Suitable procedures can include an initial disruption in osmotically-stabilizing medium followed by selective release in non-stabilizing medium. The composition of these media (pH, protective agent) and the disruption methods used (chloroform, HEW-lysozyme, EDTA, sonication) vary among specific procedures reported.

Methods for the recovery of recombinant protein from the cytoplasm, as soluble protein or refractile particles, can include disintegration of the bacterial cell by mechanical breakage. Mechanical disruption typically involves the generation of local cavitations in a liquid suspension, rapid agitation with rigid beads, sonication, or grinding of cell suspension.

HEW-lysozyme acts biochemically to hydrolyze the peptidoglycan backbone of the cell wall. Many different modifications of these methods have been used on a wide range of expression systems and are known in the art.

The proteins of this invention may be isolated and purified to substantial purity by standard techniques well known in the art, including, but not limited to, ammonium sulfate or ethanol precipitation, acid extraction, anion or cation exchange chromatography, phosphocellulose chromatography, hydrophobic interaction chromatography, affinity chromatography, nickel chromatography, hydroxylapatite chromatography, reverse phase chromatography, lectin chromatography, preparative electrophoresis, detergent solubilization, selective precipitation with such substances as column chromatography, immunopurification methods, and others. For example, proteins having established molecular adhesion properties can be reversibly fused a ligand. With the appropriate ligand, the protein can be selectively adsorbed to a purification column and then freed from the column in a relatively pure form. The fused protein is then removed by enzymatic activity. In addition, protein can be purified using immunoaffinity columns or Ni-NTA columns.

Detection of the expressed protein can be achieved by methods known in the art and include, for example, radioimmunoassays, Western blotting techniques, or immunoprecipitation.

The molecular weight of a recombinant protein can be used to isolated it from proteins of greater and lesser size using ultrafiltration through membranes of different pore size (for example, Amicon or Millipore membranes). As a first step, the protein mixture can be ultrafiltered through a membrane with a pore size that has a lower molecular weight cut-off than the molecular weight of the protein of interest. The retentate of the ultrafiltration can then be ultrafiltered against a membrane with a molecular cut off greater than the molecular weight of the protein of interest. The recombinant protein will pass through the membrane into the filtrate. The filtrate can then be chromatographed.

Recombinant proteins can also be separated from other proteins on the basis of their size, net surface charge, hydrophobicity, and affinity for ligands. In addition, antibodies raised against proteins can be conjugated to column matrices and the proteins immunopurified. All of these methods are well known in the art. It will be apparent to one of skill that chromatographic techniques can be performed at any scale and using equipment from many different manufacturers (e.g., Pharmacia Biotech).

### Active Protein or Peptide Analysis

Active proteins can have a specific activity of at least 20%, 30%, or 40%, at least 50%, 60%, or 70%, or at least 80%, 90%, or 95% that of the native protein or peptide that the sequence is derived from. Further, the substrate specificity (k_{cat}/Kₘ) is optionally substantially similar to the native protein or peptide. Typically, k_{cat}/Kₘ will be at least 30%, 40%, or 50%, that of the native protein or peptide; or at least 60%, 70%, 80%, or 90%. Methods of assaying and quantifying measures of protein and peptide activity and substrate specificity (k_{cat}/Kₘ), are well known to those of skill in the art.

The activity of a recombinant protein or peptide produced in accordance with the present invention by can be measured by any protein specific conventional or standard in vitro or in vivo assay known in the art. The activity of the Pseudomonas produced recombinant protein or peptide can be compared with the activity of the corresponding native protein to determine whether the recombinant protein exhibits substantially similar or equivalent activity to the activity generally observed in the native protein or peptide under the same or similar physiological conditions.

The activity of the recombinant protein can be compared with a previously established native protein or peptide standard activity. Alternatively, the activity of the recombinant protein or peptide can be determined in a simultaneous, or substantially simultaneous, comparative assay with the native protein or peptide. For example, an in vitro assay can be used to determine any detectable interaction between a recombinant protein or peptide and a target, e.g., between an expressed enzyme and substrate, between expressed hormone and hormone receptor, and between expressed antibody and antigen. Such detection can include the measurement of calorimetric changes, proliferation changes, cell death, cell repelling, changes in radioactivity, changes in solubility, changes in molecular weight as measured by gel electrophoresis and/or gel exclusion methods, phosphorylation abilities, antibody specificity assays such as ELISA assays, etc. In addition, in vivo assays include, but are not limited to, assays to detect physiological effects of the Pseudomonas produced protein or peptide in comparison to physiological effects of the native protein or peptide, e.g., weight gain, change in electrolyte balance, change in blood clotting time, changes in clot dissolution and the induction of antigenic response.

Generally, any in vitro or in vivo assay can be used to determine the active nature of the Pseudomonas produced recombinant protein or peptide that allows for a comparative analysis to the native protein or peptide so long as such activity is assayable. Alternatively, the proteins or peptides produced in the present invention can be assayed for the ability to stimulate or inhibit interaction between the protein or peptide and a molecule that normally interacts with the protein or peptide, e.g., a substrate or a component of the signal pathway that the native protein normally interacts. Such assays can typically include the steps of combining the protein with a substrate molecule under conditions that allow the protein or peptide to interact with the target molecule, and detect the biochemical consequence of the interaction with the protein and the target molecule.

### EXAMPLES

### Example 1

### Cloning of Expression Plasmid for Expression of Codon and Hydrophilicity Optimized CCMV Capsid Protein in Pseudomonas fluorescens

### Cloning:

Codon and hydrophobicity optimized CCMV CP nucleotide sequence was designed (SEQ ID NO:3). CCMV-CP insert (SEQ ID NO:3) containing the SpeI restriction site, ribosome binding site, CP ORF, and XhoI restriction site is excised out of a shuttle plasmid (DNA 2.0, Menlo Park, Calif.) with SpeI and XhoI. The insert is gel purified on a 1% agarose gel and ligated into the vector pDowl 169 (a medium copy plasmid with RSF1010 origin, pyrF, tac promoter, and the rrnBT1T2 terminator from pKK223-3 (PL-Pharmacia)), which is digested with SpeI, XhoI and treated with Alkaline Phosphatase (New England Biolabs) to create an expression plasmid for CCMV CP expression in Pseudomonas fluorescens (SEQ ID NO:23). The ligation product is transformed by electroporation into P. fluorescens strain DC454 (ΔpyrF RXF01414 (lsc)::lacIq1) after purification with Micro Bio-spin 6 Chromatography columns. The tranformants are plated on M9 Glucose plates after two hours shaking in LB media at 30° C. The presence of the insert is confirmed by restriction digest and sequencing of plasmid DNA isolated from single colonies.

Nucleic acid sequence of vector pDowl 169 with CCMV CP inserted at SpeI, XhoI sites for expression in Pseudomonas fluorescens (SEQ ID NO:23): CGGGTACCTGTCGA AGGGCTGGAGACATTCCCGGAAACGCTGATGAAGCTGTTCAACGGCGAGAACTTCG GGAAGTTGGTGCTCAAAGTCAGCTGACACACCACAAAAACAAATGTGGGAGCTGGC TTGCCTGCGATGCAAGCAACTCGGTTTCTAAGTGATACCGAGTTGATACTATCGCAG GCAAGCCAGCTCCCACATTTTTTGCTCACTCTAAAAATCAGGCGATCTCGGCGACGA CCGCTGCCAACGCTTTTGCAGGATCCGCCGCCTGGCTGATCGGCCGGCCGATCACCA GGTAGTCAGAGCCCGCATCCAGGGCCTGGCGCGGGGTCAGGATACGGCGCTGGTCA TCCTGGGCGCTGCCGGTAGGACGGATACCCGGTGTCACCAGTTGCAGCGACGGATG TGCGTTTTTCAGGGCCTGGGCTTCCAGGGCTGAGCACACCAGGCCGTCGAGGCCGGC TTTCTGCGCCAGGGCTGCCAGGCGCAACACTTGCACCTGCGGCTCGATATCCAGGCC AATGCCCGCCAGGTCTTCGCGCTCCATGCTGGTGAGCACGGTCACGCCGATCAACAA CGGTTTGGGGCCGCTGCGCTGTTCCAGCACTTCGCGGCAGGCGCTCATCATGCGCAG GCCACCGGAGCAGTGCACATTGACCATCCACACGCCCATCTCGGCCGCGGCTTTGAC GGCCATCGCCGTGGTGTTGGGGATGTCATGGAATTTGAGGTCGAGGAACACTTCGA AGCCTTTGTCCCGCAGGGTGCCGACGATTTCCGCCGCGCAACTGGTGAACAATTCCT TGCCGACCTTGACCCGGCAAAGCTTGGGGTCCAACTGGTCAGCCAGCTTCAGTGCGG CGTCACGGGTGGGGTAATCCAGGGCGACGATGATAGGAGTCTGGCAGACGGACATT GGAATGGGCTCTCAGGCAAGTCGAAATCGGCGCGGATTGTAGCGCAAGCAGCGCCG TTGCGGGATCCGATGATCGGTAAATACCGATCAAGCGCCCAATACCGGCGATTCAA GGCAATTGTGAGCGCTCACAATTTATTCTGAAATGAGCTGTTGACAATTAATCATCG GCTCGTATAATGTGTGGAATTGTGAGCGGATAACAATTTCACACAGGAAACAGAAT TTTAATCTACTAGTAGGAGGTAACTTATGTCCACTGTCGGCACTGGCAAATTGACCC GTGCACAACGTCGTGCCGCTGCCCGCAAGAACAAGCGGAAGACCCGGGTGGTCCAG CCGGTCATCGTGGAGCCGATTGCCTCCGGCCAGGGCAAGGCCATTAAAGCCTGGAC GGGCTACAGCGTCAGCAAATGGACGGCCAGCTGCGCGGCTGCCGAGGCCAAGGTCA CCAGTGCGATTACGATCAGCCTCCCCAACGAATTGAGCAGCGAGCGGAACAAGCAA CTGAAGGTCGGCCGGGTCCTGCTGTGGCTGGGTCTGCTGCCGAGTGTGTCCGGCACC GTCAAGTCCTGCGTGACGGAAACCCAGACTACCGCGGCAGCTTCCTTTCAGGTTGCC CTGGCAGTGGCTGATAACTCCAAAGACGTCGTTGCGGCCATGTACCCAGAGGCCTTC AAGGGCATCACCCTGGAGCAGCTGACCGCAGACCTGACCATTTATCTCTACAGCAGT GCCGCGCTGACGGAAGGCGACGTCATCGTCCATTTGGAAGTGGAACACGTCCGCCC GACGTTCGATGACTCGTTCACTCCGGTGTATTGATAATAGCTCGAGCCCAAAACGAA AGGCTCAGTCGACAGACTGGGCCTTTCGTTTTATCTGTTGTTTGTCGGTGAACGCTCT CCTGAGTAGGACAAATCCGCCGGGAGCGGATTTGAACGTTGCGAAGCAACGGCCCG GAGGGTGGCGGGCAGGACGCCCGCCATAAACTGCCAGGCATCAAATTAAGCAGAA GGCCATCCTGACGGATGGCCTTTTTGCGTTTCTACAAACTCTTTTGGCATATGGGGG ATCTGGCTGCAGGAGCAGAAGAGCATACATCTGGAAGCAAAGCCAGGAAAGCGGC CTATGGAGCTGTGCGGCAGCGCTCAGTAGGCAATTTTTCAAAATATTGTTAAGCCTT TTCTGAGCATGGTATTTTTCATGGTATTACCAATTAGCAGGAAAATAAGCCATTGAA TATAAAAGATAAAAATGTCTTGTTTACAATAGAGTGGGGGGGGTCAGCCTGCCGCCT TGGGCCGGGTGATGTCGTACTTGCCCGCCGCGAACTCGGTTACCGTCCAGCCCAGCG CGACCAGCTCCGGCAACGCCTCGCGCACCCGCTGGCGGCGCTTGCGCATGGTCGAA CCACTGGCCTCTGACGGCCAGACATAGCCGCACAAGGTATCTATGGAAGCCTTGCC GGTTTTGCCGGGGTCGATCCAGCCACACAGCCGCTGGTGCAGCAGGCGGGCGGTTT CGCTGTCCAGCGCCCGCACCTCGTCCATGCTGATGCGCACATGCTGGCCGCCACCCA TGACGGCCTGCGCGATCAAGGGGTTCAGGGCCACGTACAGGCGCCCGTCCGCCTCG TCGCTGGCGTACTCCGACAGCAGCCGAAACCCCTGCCGCTTGCGGCCATTCTGGGCG ATGATGGATACCTTCCAAAGGCGCTCGATGCAGTCCTGTATGTGCTTGAGCGCCCCA CCACTATCGACCTCTGCCCCGATTTCCTTTGCCAGCGCCCGATAGCTACCTTTGACCA CATGGCATTCAGCGGTGACGGCCTCCCACTTGGGTTCCAGGAACAGCCGGAGCTGC CGTCCGCCTTCGGTCTTGGGTTCCGGGCCAAGCACTAGGCCATTAGGCCCAGCCATG GCCACCAGCCCTTGCAGGATGCGCAGATCATCAGCGCCCAGCGGCTCCGGGCCGCT GAACTCGATCCGCTTGCCGTCGCCGTAGTCATACGTCACGTCCAGCTTGCTGCGCTT GCGCTCGCCCCGCTTGAGGGCACGGAACAGGCCGGGGGCCAGACAGTGCGCCGGGT CGTGCCGGACGTGGCTGAGGCTGTGCTTGTTCTTAGGCTTCACCACGGGGCACCCCC TTGCTCTTGCGCTGCCTCTCCAGCACGGCGGGCTTGAGCACCCCGCCGTCATGCCGC CTGAACCACCGATCAGCGAACGGTGCGCCATAGTTGGCCTTGCTCACACCGAAGCG GACGAAGAACCGGCGCTGGTCGTCGTCCACACCCCATTCCTCGGCCTCGGCGCTGGT CATGCTCGACAGGTAGGACTGCCAGCGGATGTTATCGACCAGTACCGAGCTGCCCC GGCTGGCCTGCTGCTGGTCGCCTGCGCCCATCATGGCCGCGCCCTTGCTGGCATGGT GCAGGAACACGATAGAGCACCCGGTATCGGCGGCGATGGCCTCCATGCGACCGATG ACCTGGGCCATGGGGCCGCTGGCGTTTTCTTCCTCGATGTGGAACCGGCGCAGCGTG TCCAGCACCATCAGGCGGCGGCCCTCGGCGGCGCGCTTGAGGCCGTCGAACCACTC CGGGGCCATGATGTTGGGCAGGCTGCCGATCAGCGGCTGGATCAGCAGGCCGTCAG CCACGGCTTGCCGTTCCTCGGCGCTGAGGTGCGCCCCAAGGGCGTGCAGGCGGTGA TGAATGGCGGTGGGCGGGTCTTCGGCGGGCAGGTAGATCACCGGGCCGGTGGGCAG TTCGCCCACCTCCAGCAGATCCGGCCCGCCTGCAATCTGTGCGGCCAGTTGCAGGGC CAGCATGGATTTACCGGCACCACCGGGCGACACCAGCGCCCCGACCGTACCGGCCA CCATGTTGGGCAAAACGTAGTCCAGCGGTGGCGGCGCTGCTGCGAACGCCTCCAGA ATATTGATAGGCTTATGGGTAGCCATTGATTGCCTCCTTTGCAGGCAGTTGGTGGTT AGGCGCTGGCGGGGTCACTACCCCCGCCCTGCGCCGCTCTGAGTTCTTCCAGGCACT CGCGCAGCGCCTCGTATTCGTCGTCGGTCAGCCAGAACTTGCGCTGACGCATCCCTT TGGCCTTCATGCGCTCGGCATATCGCGCTTGGCGTACAGCGTCAGGGCTGGCCAGCA GGTCGCCGGTCTGCTTGTCCTTTTGGTCTTTCATATCAGTCACCGAGAAACTTGCCGG GGCCGAAAGGCTTGTCTTCGCGGAACAAGGACAAGGTGCAGCCGTCAAGGTTAAGG CTGGCCATATCAGCGACTGAAAAGCGGCCAGCCTCGGCCTTGTTTGACGTATAACCA AAGCCACCGGGCAACCAATAGCCCTTGTCACTTTTGATCAGGTAGACCGACCCTGAA GCGCTTTTTTCGTATTCCATAAAACCCCCTTCTGTGCGTGAGTACTCATAGTATAACA GGCGTGAGTACCAACGCAAGCACTACATGCTGAAATCTGGCCCGCCCCTGTCCATGC CTCGCTGGCGGGGTGCCGGTGCCCGTGCCAGCTCGGCCCGCGCAAGCTGGACGCTG GGCAGACCCATGACCTTGCTGACGGTGCGCTCGATGTAATCCGCTTCGTGGCCGGGC TTGCGCTCTGCCAGCGCTGGGCTGGCCTCGGCCATGGCCTTGCCGATTTCCTCGGCA CTGCGGCCCCGGCTGGCCAGCTTCTGCGCGGCGATAAAGTCGCACTTGCTGAGGTCA TCACCGAAGCGCTTGACCAGCCCGGCCATCTCGCTGCGGTACTCGTCCAGCGCCGTG CGCCGGTGGCGGCTAAGCTGCCGCTCGGGCAGTTCGAGGCTGGCCAGCCTGCGGGC CTTCTCCTGCTGCCGCTGGGCCTGCTCGATCTGCTGGCCAGCCTGCTGCACCAGCGC CGGGCCAGCGGTGGCGGTCTTGCCCTTGGATTCACGCAGCAGCACCCACGGCTGAT AACCGGCGCGGGTGGTGTGCTTGTCCTTGCGGTTGGTGAAGCCCGCCAAGCGGCCAT AGTGGCGGCTGTCGGCGCTGGCCGGGTCGGCGTCGTACTCGCTGGCCAGCGTCCGG GCAATCTGCCCCCGAAGTTCACCGCCTGCGGCGTCGGCCACCTTGACCCATGCCTGA TAGTTCTTCGGGCTGGTTTCCACTACCAGGGCAGGCTCCCGGCCCTCGGCTTTCATGT CATCCAGGTCAAACTCGCTGAGGTCGTCCACCAGCACCAGACCATGCCGCTCCTGCT CGGCGGGCCTGATATACACGTCATTGCCCTGGGCATTCATCCGCTTGAGCCATGGCG TGTTCTGGAGCACTTCGGCGGCTGACCATTCCCGGTTCATCATCTGGCCGGTGGTGG CGTCCCTGACGCCGATATCGAAGCGCTCACAGCCCATGGCCTTGAGCTGTCGGCCTA TGGCCTGCAAAGTCCTGTCGTTCTTCATCGGGCCACCAAGCGCAGCCAGATCGAGCC GTCCTCGGTTGTCAGTGGCGTCAGGTCGAGCAAGAGCAACGATGCGATCAGCAGCA CCACCGTAGGCATCATGGAAGCCAGCATCACGGTTAGCCATAGCTTCCAGTGCCACC CCCGCGACGCGCTCCGGGCGCTCTGCGCGGCGCTGCTCACCTCGGCGGCTACCTCCC GCAACTCTTTGGCCAGCTCCACCCATGCCGCCCCTGTCTGGCGCTGGGCTTTCAGCC ACTCCGCCGCCTGCGCCTCGCTGGCCTGCTGGGTCTGGCTCATGACCTGCCGGGCTT CGTCGGCCAGTGTCGCCATGCTCTGGGCCAGCGGTTCGATCTGCTCCGCTAACTCGT TGATGCCTCTGGATTTCTTCACTCTGTCTATTGCGTTCGTGGTCTATTGCGTTCGTGGT CTATTGCCTCCCGGTATTCCTGTAAGTCGATGATCTGGGCGTTGGCGGTGTCGATGTT CAGGGCCACGTCTGCCCGGTCGGTGCGGATGCCCCGGCCTTCCATCTCCACCACGTT CGGCCCCAGGTGAACACCGGGCAGGCGCTCGATGCCCTGCGCCTCAAGTGTTCTGTG GTCAATGCGGGCGTCGTGGCCAGCCCGCTCTAATGCCCGGTTGGCATGGTCGGCCCA TGCCTCGCGGGTCTGCTCAAGCCATGCCTTGGGCTTGAGCGCTTCGGTCTTCTGTGCC CCGCCCTTCTCCGGGGTCTTGCCGTTGTACCGCTTGAACCACTGAGCGGCGGGCCGC TCGATGCCGTCATTGATCCGCTCGGAGATCATCAGGTGGCAGTGCGGGTTCTCGCCG CCACCGGCATGGATGGCCAGCGTATACGGCAGGCGCTCGGCACCGGTCAGGTGCTG GGCGAACTCGGACGCCAGCGCCTTCTGCTGGTCGAGGGTCAGCTCGACCGGCAGGG CAAATTCGACCTCCTTGAACAGCCGCCCATTGGCGCGTTCATACAGGTCGGCAGCAT CCCAGTAGTCGGCGGGCCGCTCGACGAACTCCGGCATGTGCCCGGATTCGGCGTGC AAGACTTCATCCATGTCGCGGGCATACTTGCCTTCGCGCTGGATGTAGTCGGCCTTG GCCCTGGCCGATTGGCCGCCCGACCTGCTGCCGGTTTTCGCCGTAAGGTGATAAATC GCCATGCTGCCTCGCTGTTGCTTTTGCTTTTCGGCTCCATGCAATGGCCCTCGGAGAG CGCACCGCCCGAAGGGTGGCCGTTAGGCCAGTTTCTCGAAGAGAAACCGGTAAGTG CGCCCTCCCCTACAAAGTAGGGTCGGGATTGCCGCCGCTGTGCCTCCATGATAGCCT ACGAGACAGCACATTAACAATGGGGTGTCAAGATGGTTAAGGGGAGCAACAAGGC GGCGGATCGGCTGGCCAAGCTCGAAGAACAACGAGCGCGAATCAATGCCGAAATTC AGCGGGTGCGGGCAAGGGAACAGCAGCAAGAGCGCAAGAACGAAACAAGGCGCAA GGTGCTGGTGGGGGCCATGATTTTGGCCAAGGTGAACAGCAGCGAGTGGCCGGAGG ATCGGCTCATGGCGGCAATGGATGCGTACCTTGAACGCGACCACGACCGCGCCTTGT TCGGTCTGCCGCCACGCCAGAAGGATGAGCCGGGCTGAATGATCGACCGAGACAGG CCCTGCGGGGCTGCACACGCGCCCCCACCCTTCGGGTAGGGGGAAAGGCCGCTAAA GCGGCTAAAAGCGCTCCAGCGTATTTCTGCGGGGTTTGGTGTGGGGTTTAGCGGGCT TTGCCCGCCTTTCCCCCTGCCGCGCAGCGGTGGGGCGGTGTGTAGCCTAGCGCAGCG AATAGACCAGCTATCCGGCCTCTGGCCGGGCATATTGGGCAAGGGCAGCAGCGCCC CACAAGGGCGCTGATAACCGCGCCTAGTGGATTATTCTTAGATAATCATGGATGGAT TTTTCCAACACCCCGCCAGCCCCCGCCCCTGCTGGGTTTGCAGGTTTGGGGGCGTGA CAGTTATTGCAGGGGTTCGTGACAGTTATTGCAGGGGGGCGTGACAGTTATTGCAGG GGTTCGTGACAGTTAGTACGGGAGTGACGGGCACTGGCTGGCAATGTCTAGCAACG GCAGGCATTTCGGCTGAGGGTAAAAGAACTTTCCGCTAAGCGATAGACTGTATGTA AACACAGTATTGCAAGGACGCGGAACATGCCTCATGTGGCGGCCAGGACGGCCAGC CGGGATCGGGATACTGGTCGTTACCAGAGCCACCGACCCGAGCAAACCCTTCTCTAT CAGATCGTTGACGAGTATTACCCGGCATTCGCTGCGCTTATGGCAGAGCAGGGAAA GGAATTGCCGGGCTATGTGCAACGGGAATTTGAAGAATTTCTCCAATGCGGGCGGCT GGAGCATGGCTTTCTACGGGTTCGCTGCGAGTCTTGCCACGCCGAGCACCTGGTCGC TTTCAGCTGTAAGCGTCGCGGTTTCTGCCCGAGCTGTGGGGCGCGGCGGATGGCCGA AAGTGCCGCCTTGCTGGTTGATGAAGTACTGCCTGAACAACCCATGCGTCAGTGGGT GTTGAGCTTCCCGTTTCAGCTGCGTTTCCTGTTTGGGGTCGTTTGCGGGAAGGGGCG GAATCCTACGCTAAGGCTTTGGCCAGCGATATTCTCCGGTGAGATTGATGTGTTCCC AGGGGATAGGAGAAGTCGCTTGATATCTAGTATGACGTCTGTCGCACCTGCTTGATC GCGGCCCACCGCGGCGGGAAGCAGGTGCGATTTTCGCGAAGGCATGCCCGTCACCA CGTCGAAAAACAAAATCACCAGATTCTCCGCCTCTGACAGGCAACCAGTCAGAATG CGATTCACCAAAAAAAATATTAGTTCGATTCAATGGAGGTTCCTTCAGTTTTCTGAT GAAGCGCGAATATAGAGAAATATCCCGAATGTGCAGTTAACGAATTCCGGCTGTCC GGCGTTTTCGTGGAGCCCGAACAGCGAGGCCGAGGGGTCGCCGGTATGCTGCTGCG GGCGTTGCCGGCGGGTTTATTGCTCGTGATGATCGTCCGACAGATTCCAACGGGAAT CTGGTGGATGCGCATCTTCATCCTCGGCGCACTTAATATTTCGCTATTCTGGAGCTTG TTGTTTATTTCGGTCTACCGCCTGCCGGGCGGGGTCGCGGCGACGGTAGGCGCTGTG CAGCCGCTGATGGTCGTGTTCATCTCTGCCGCTCTGCTAGGTAGCCCGATACGATTG ATGGCGGTCCTGGGGGCTATTTGCGGAACTGCGGGCGTGGCGCTGTTGGTGTTGACA CCAAACGCAGCGCTAGATCCTGTCGGCGTCGCAGCGGGCCTGGCGGGGGCGGTTTC CATGGCGTTCGGAACCGTGCTGACCCGCAAGTGGCAACCTCCCGTGCCTCTGCTCAC CTTTACCGCCTGGCAACTGGCGGCCGGAGGACTTCTGCTCGTTCCAGTAGCTTTAGT GTTTGATCCGCCAATCCCGATGCCTACAGGAACCAATGTTCTCGGCCTGGCGTGGCT CGGCCTGATCGGAGCGGGTTTAACCTACTTCCTTTGGTTCCGGGGGATCTCGCGACT CGAACCTACAGTTGTTTCCTTACTGGGCTTTCTCAGCCCGGGGACCGCCGTGTTGCTA GGATGGTTGTTCTTGGATCAGACGCTGAGTGCGCTTCAAATCATCGGCGTCCTGCTC GTGATCGGGAGTGTCTGGCTGGGCCAACGTTCCAACCGCACTCCTAGGGCGCGTATA GCTTGCCGGAAGTCGCCTTGACCCGCATGGCATAGGCCTATCGTTTCCACGATCAGC GAT.

### Protein Expression:

Single transformants are inoculated into 50 ml M9 Glucose media and grown overnight. P. fluorescens cultures of 3.0-5.0 OD600 are used to inoculate 200 ml shake flask media. Shake flask cultures are incubated at 30° C. with 300 rpm shaking overnight. Overnight cultures of 15.0-20.0 OD600 are induced with 300 μM isopropyl-β-D-thiogalactopyranoside (IPTG).

### Example 2

### Introduction of Restriction Sites into Loops of Codon and Hydrophilicity Optimized CCMV Capsid Protein

Site-directed mutagenesis reactions are carried out using Quikchange II-XL (Stratagene, TX) according to manufacturer's protocol. The P. fluorescens expression plasmid harboring codon-optimized CCMV-CP (SEQ ID NO:23) serves as a template. Resulting plasmids with introduced restriction sites are transformed into P. fluorescens strain DC454 (ΔpyrF RXF01414 (lsc)::lacIq1) by electroporation after purification with Micro Bio-spin 6. Protein expression is performed as described in Example 1.

Primers for Introduction of Blunt-End Cutting Restriction Site AfeI I onto 63 Loop:

CCMV-AfeI-63-F (SEQ ID NO:24):
5′-TGCGCGGCTGCCGAGAGCGCTGCCAAGGTCACCAGT-3′
CCMV-AfeI-63-R (SEQ ID NO:25):
5′-ACTGGTGACCTTGGCAGCGCTCTCGGCAGCCGCGCA-3′

Primers for Introduction of 3′-Overhang-Cutting Restriction Site PvuI into 102 Loop:

CCMV-PvuI-102-F (SEQ ID NO:26):
5′-CTGCCGAGTGTGTCCCGATCGGGCACCGTCAAGTCC-3′
CCMV-PvuI-102-R (SEQ ID NO:27):
5′-GGACTTGACGGTGCCCGATCGGGACACACTCGGCAG-3′

Primers for Introduction of 5′-Overhang-Cutting Restriction Site BglII into 114 Loop:

CCMV-Bgl II-114-F (SEQ ID NO:28):
5′- ACGGAAACCCAGACTAGATCTACCGCGGCAGCTTCC -3′
CCMV-Bgl II-114-R (SEQ ID NO:29):
5′- GGAAGCTGCCGCGGTAGATCTAGTCTGGGTTTCCGT -3′

Primers for Introduction of 5′-Overhang-Cutting Restriction Site XbaI into 129 Loop:

CCMV-XbaI-129-F (SEQ ID NO:30):
5′- GCAGTGGCTGATAACTCAAGATCCAAAGACGTCGTT -3′
CCMV-XbaI-129-R (SEQ ID NO:31):
5′- AACGACGTCTTTGGATCTTGAGTTATCAGCCACTGC -3′

Primers for Introduction of 5′-Overhang-Cutting Restriction Site NheI into 160 Loop:

CCMV-NheI-160-F (SEQ ID NO:32):
5′- CCATTTATCTCTACAGCGCTAGCAGTGCCGCGCTGACG -3′
CCMV-NheI-160-R (SEQ ID NO:33):
5′- CGTCAGCGCGGCACTGCTAGCGCTGTAGAGATAAATGG -3′

### Example 3

### Restriction Digestion-Based Cloning and Expression of Flu Vaccine M2e Peptide Fused to the 129 Surface Loop of Codon and Hydrophilicity Optimized CCMV Capsid Protein

### Peptide Synthesis:

The insert is synthesized by over-lapping DNA oligonucleotides described below with the thermocycling program detailed below:

PCR PROTOCOL

| Reaction Mix (100 μL total volume) | | | Thermocycling Steps | | | |
|---|---|---|---|---|---|---|
| 10 | μL | 10X PT HIFI buffer* | Step 1 | 1 Cycle | 2 minute | 94° C. |
| 4 | μL | 50 mM MgSO₄* | Step 2 | 35 Cycles | 30 second | 94° C. |
| 2 | μL | 10 mM dNTPs* | | | 30 second | 55° C. |
| 0.25 | ng | Each Primer | | | 1 minute | 68° C. |
| 1-5 | ng | Template DNA | Step 3 | 1 Cycle | 10 minute | 70° C. |
| 1 | μL | PT HIFI Taq DNA Polymerase* | Step 4 | 1 Cycle | Maintain | 4° C. |

| | |
|---|---|
| Remainder | Distilled De-ionized H₂O (ddH₂O) |

*(from Invitrogen Corp, Carlsbad, CA)

The PCR product is purified with Qiaquick® PCR purification kit (Qiagen), digested with XbaI (NEB) and purified again with Qiaquick® kit before ligating into XbaI restricted CCMV CP P. fluorescens expression vector containing XbaI restriction site in the 129 loop (from Example 2) with T4 DNA ligase (NEB). The ligation product is transformed by electroporation into P. fluorescens strain DC454 (ΔpyrF RXF01414 (lsc)::lacIq1) after purification with Micro Bio-spin 6 Chromatography columns (Biorad). The tranformants are plated on M9 Glucose plate (Teknova) after two hours shaking in LB media at 30° C. The plates are incubated at 30° C. for 48 hours. The presence of the insert is confirmed by restriction digest and sequencing. Protein expression is performed as described in Example 1.

### Amino Acid Sequence of Flu Vaccine M2e-1 Peptide:

| | |
|---|---|
| SLLTEVETPIRNEWGCRCNDSSD | (SEQ ID NO:34) |

Codon-Optimized Nucleic Acid Sequence for M2e-1:

(SEQ ID NO:35)

AGCTTGTTGACTGAAGTTGAAACGCCAATCCGTAATGAATGGGGCTGCCG
GTGCAACGATAGTTCCGAC

Primers for Cloning into the Restriction Site XbaI in the 129 Surface Loop:

M2e-CCMV129-F (SEQ ID NO:36):
CGTCTAGAAGCTTGTTGACTGAAGTTGAAACGCCAATCCGTAATGAATGG
GG
M2e-CCMV129-R (SEQ ID NO:37):
CGTCTAGAGTCGGAACTATCGTTGCACCGGCAGCCCCATTCATTACGGAT
TG

### Example 4

### SOE (Splicing by Overlapping Extension) Based Cloning and Expression of Flu Vaccine M2e Peptide Fused to the 129 Surface Loop of Codon and Hydrophilicity Optimized CCMV Capsid Protein

Step 1: P. fluorescens expression plasmid harboring codon-optimized CCMV-CP (SEQ ID NO:23) is used as PCR template. Reaction 1 (see FIG. 10) uses primers Coop-CCMV-F and PCP-CCMV129-SOE-R primers. Reaction 2 uses primers Coop-CCMV-R and PCP-CCMV129-SOE-F. PCR is carried out according to the thermocycling protocols described above.

Step 2: Products from reactions 1 and 2 are used as PCR templates. Coop-CCMV-F and Coop-CCMV-R primers are used to amplify final PCR product.

Final PCR product is then digested by SpeI and XhoI and subcloned into P. fluorescens expression vector pDowl 169 at SpeI and XhoI. The ligation product is transformed by electroporation into P. fluorescens strain DC454 after purification with Micro Bio-spin 6 Chromatography columns (Biorad). The tranformants are plated on M9 Glucose plate (Teknova) after two hours shaking in LB media at 30° C. The plates are incubated at 30° C. for 48 hours. The presence of the insert is confirmed by restriction digest and sequencing. Protein expression is carried out as described in Example 1.

Primers for SOE Based M2e-CCMV Fusion:

Coop-CCMV-F (SEQ ID NO:38):
5′- GGACTAGTAGGAGGTAACTTATGTCCACTGTCGGCACTGG -3′
Coop-CCMV-R (SEQ ID NO:39):
5′- CCGCTCGAGTCATTACTATTATCAATACACCGGAG -3′
M2e-CCMV129-SOE-F (SEQ ID NO:40):
5′- CAATCCGTAATGAATGGGGCTGCCGGTGCAACGATAGTTCCGACtc
caaagacgtcgttgcgg -3′
M2e-CCMV129-SOE-R (SEQ ID NO:41):
5′- CCCCATTCATTACGGATTGGCGTTTCAACTTCAGTCAACAAGCTTC
TAGACGGTTATCAGCCACTGCCAGG -3′

### Example 5

### Restriction Digestion-Based Cloning and Expression of Flu Vaccine M2e-1 Peptide in Dual Surface Loops 63 and 129 on Codon and Hydrophilicity Optimized CCMV Capsid Protein

### Cloning:

A blunt-cutting restriction site AfeI is introduced into the surface loop 63 of the P. fluorescens expression plasmid harboring codon-optimized CCMV capsid protein gene with a monomeric M2e-1 fused at the 129 surface loop as described in Examples 2 and 3. The AfeI restriction site is introduced using primers CCMV-AfeI-63-F (SEQ ID NO:24) and CCMV-AfeI-63-R (SEQ ID NO:25). M2e-1 is synthesized by PCR. Primers to synthesize blunt-ended M2e-1 DNA fragment are M2e-1-blunt-For (tcactcttgacagaggtagaaacaccgata cgtaatgaatggggc SEQ ID NO:42) and M2e-1-blunt-Rev (atctgaagaatcattacaacgacagcccca ttcattacgtatcgg SEQ ID NO:43).

PCR synthesis is carried out as described but with Pfu Turbo Polymerase in lieu of Taq Polymerase to create blunt ends. The M2e-1 PCR fragment is then treated with the Klenow fragment of DNA polymerase I to yield blunt-ended M2e-1 DNA insert. The insert is then ligated into the CCMV CP expression plasmid with the AfeI restriction site in the surface loop 63 after restriction with AfeI. The resulting ligation product is transformed into P. fluorescens strain DC454 (ΔpyrF RXF01414 (lsc)::lacIq1) by electroporation after purification with Micro Bio-spin 6. The presence and integrity of the dual inserts are verified by restriction digest and sequencing. Protein expression is performed as described in Example 1.

### Example 6

### SOE Based Cloning and Expression of Anthrax Vaccine Peptide PA1 Fused to Codon and Hydrophilicity Optimized CCMV Capsid Protein

PA1-129-SOE-F (SEQ ID NO:44):
5′- CCAGCGCCGGTCCAACCGTGCCCGACCGCGACAACGATGGCATCCC
CGACtccaaagacgtcgttgcgg -3′
PA1-129-SOE-R (SEQ ID NO:45):
5′- TCGGGCACGGTTGGACCGGCGCTGGTGGAGCGTTTCTTGCGACTAT
TACTGTTATCAGCCACTGCCAGG -3′

Step 1: P. fluorescens expression plasmid harboring codon-optimized CCMV-CP (SEQ ID NO:23) was used as PCR template. Coop-CCMV-F and PA1-129-SOE-R primers were used in the reaction 1. Coop CCMV-R and PA1-129-SOE-F primers were used in the reaction 2. PCRs were carried out according to the thermocycling protocols described above.

Step 2: PCR products from the reaction 1 and 2 were used as PCR templates for this reaction. Coop-CCMV-F and Coop-CCMV-R primers were used to amplify out final PCR product.

Final PCR product was then digested by SpeI and XhoI and subcloned into P. fluorescens expression vector pDowl 169 at SpeI and XhoI. The ligation product was transformed by electroporation into P. fluorescens strain DC454 after purification with Micro Bio-spin 6 Chromatography columns (Biorad). The tranformants were plated on M9 Glucose plate (Teknova) after two hours shaking in LB media at 30° C. The plates were incubated at 30° C. for 48 hours. The presence of the insert was confirmed by restriction digest and sequencing. Protein expression was performed as described above. Expression of soluble anthrax vaccine peptide PA1 fused to the CCMV capsid protein in P. fluorescens is shown in FIG. 9.

### Example 7

### Restriction Digestion-Based Cloning and Expression of Anthrax Vaccine Peptide PA1 Fused to Codon and Hydrophilicity Optimized CCMV Capsid Protein

### Peptide Sequence of Anthrax Vaccine Peptide PA1:

| | |
|---|---|
| SNSRKKRSTSAGPTVPDRDNDGIPD | (SEQ ID NO:46) |

### Codon-Optimized Nucleic Acid Sequence for PA1:

(SEQ ID NO:47)

AGTAATAGTCGCAAGAAACGCTCCACCAGCGCCGGTCCAACCGTGCCCGA
CCGCGACAACGATGGCATCCCCGAC

The PA1 DNA insert is synthesized by PCR using primers PA1-129-XbaI-F and PA1-129-XbaI-R using cloning techniques described above. The PCR product is purified with Qiaquick PCR purification kit (Qiagen), digested with XbaI (NEB) and purified again with Qiaquick kit before ligating into XbaI restricted CCMV CP P. fluorescens expression vector containing XbaI restriction site in the 129 loop (from Example 2) with T4 DNA ligase (NEB). The ligation product is transformed by electroporation into P. fluorescens strain DC454 (ΔpyrF RXF01414 (lsc)::lacIq1) after purification with Micro Bio-spin 6 Chromatography columns (Biorad). The tranformants are plated on M9 Glucose plate (Teknova) after two hours shaking in LB media at 30° C. The plates are incubated at 30° C. for 48 hours. The presence of the insert is confirmed by restriction digest and sequencing. Protein expression is performed as described in Example 1.

PA1-129-XbaI-F (SEQ ID NO:48):
5′- CTTCTAGAAGTAATAGTCGCAAGAAACGCTCCACCAGCGCCGGTCC
AACCGTGCCCGA -3′
PA1-129-XbaI-R (SEQ ID NO:49):
5′- CTTCTAGAGTCGGGGATGCCATCGTTGTCGCGGTCGGGCACGGTTG
GACCGGCGCTGG -3′

### Example 8

### Restriction Digestion-Based Cloning and Expression of Anthrax Vaccine Peptide PA4 Fused to Codon and Hydrophilicity Optimized CCMV Capsid Protein

### Peptide Sequence of Anthrax Vaccine Peptide PA4:

| | |
|---|---|
| RQDGKTFIDFKKYNDKLPLYISNPN | (SEQ ID NO:50) |

### Codon-Optimized Nucleic Acid Sequence of PA4:

(SEQ ID NO:51)

CGCCAGGATGGTAAGACGTTCATCGACTTTAAGAAATACAACGACAAGCT
GCCCCTGTATATTTCCAACCCTAAT

The PA4 DNA insert is synthesized by PCR using primers PA4-129-XbaI-F and PA4-129-XbaI-R using cloning techniques described above. Protein expression is performed as described above.

PA4-129-XbaI-F (SEQ ID NO:52):
CTTCTAGACGCCAGGATGGTAAGACGTTCATCGACTTTAAGAAATACAAC
GACAAGCT
PA4-129-XbaI-R (SEQ ID NO:53):
CTTCTAGAATTAGGGTTGGAAATATACAGGGGCAGCTTGTCGTTGTATTT
CTTAAAGT

### Example 9

### SOE Based Cloning and Expression of Anthrax Vaccine Peptide PA4 Fused to Codon and Hydrophilicity Optimized CCMV Capsid Protein

PA4129-SOE-F (SEQ ID NO:54):
5′- ACTTTAAGAAATACAACGACAAGCTGCCCCTGTATATTTCCAACCC
TAATTCCAAAGACGTCGTTGCGG -3′
PA4-129-SOE-R (SEQ ID NO:55):
5′- AGCTTGTCGTTGTATTTCTTAAAGTCGATGAACGTCTTACCATCCT
GGCGGTTATCAGCCACTGCCAGG -3′

Step 1: P. fluorescens expression plasmid harboring codon-optimized CCMV-CP (SEQ ID NO:23) was used as PCR template. Coop-CCMV-F and PA4-129-SOE-R primers were used in reaction 1. Coop CCMV-R and PA4-129-SOE-F primers were used in reaction 2. PCRs were carried out according to the thermocycling protocols described above.

Step 2: PCR products from reactions 1 and 2 were used as PCR templates for this reaction. Coop-CCMV-F and Coop-CCMV-R primers were used to amplify out final PCR product.

Final PCR product was then digested by SpeI and XhoI and subcloned into P. fluorescens expression vector pDow1169 at SpeI and XhoI. The ligation product was transformed by electroporation into P. fluorescens strain DC454 after purification with Micro Bio-spin 6 Chromatography columns (Biorad). The tranformants were plated on M9 Glucose plate (Teknova) after two hours shaking in LB media at 30° C. The plates were incubated at 30° C. for 48 hours. The presence of the insert was confirmed by restriction digest and sequencing. Protein expression was carried out as described in the Example 1.

### Example 10

### Expression of Codon-Unoptimized Hydrophilicity-Optimized CCMV Coat Protein in P. fluorescens

The CCMV coat protein was amplified by PCR from CCMV RNA3 plasmid (pCC3) using primers CCMV-SpeI-For (CTACTAGTAGGAGGTAACTTATGTCTACAGT CGGA, SEQ ID NO:56) and CCMV-XhoI-Rev (CCGCTCGAGTCATTAATACACCGGA GTGA, SEQ ID NO:57). PCR was performed using the following protocol:

PCR PROTOCOL

| Reaction Mix (100 μL total volume) | | | Thermocycling Steps | | | |
|---|---|---|---|---|---|---|
| 10 | μL | 10X PT HIFI buffer* | Step 1 | 1 Cycle | 2 minutes | 94° C. |
| 4 | μL | 50 mM MgSO₄* | Step 2 | 35 Cycles | 30 seconds | 94° C. |
| 2 | μL | 10 mM dNTPs* | | | 30 seconds | 55° C. |
| 0.25 | ng | Each Primer | | | 1 minute | 68° C. |
| 1-5 | ng | Template DNA | Step 3 | 1 Cycle | 10 minute | 70° C. |
| 1 | μL | PT HIFI Taq DNA Polymerase* | Step 4 | 1 Cycle | Maintain | 4° C. |

| | |
|---|---|
| Remainder | Distilled De-ionized H₂O (ddH₂O) |

*(from Invitrogen Corp, Carlsbad, CA, USA, hereinafter “Invitrogen”)

The PCR product was purified with Qiaquick PCR purification kit (Qiagen), digested with SpeI and XhoI (NEB) and purified again before ligating into XbaI restricted CCMV CP expression vector with T4 DNA ligase (NEB). The ligation product was transformed by electroporation into P. fluorescens strain DC454 after purification with Micro Bio-spin 6 Chromatography columns (Biorad). The tranformants were plated on M9 Glucose plate (Teknova) after two hours shaking in LB media at 30° C. The plates were incubated at 30° C. for 48 hours. The presence of the insert was confirmed by restriction digest and sequencing. Protein Expression was performed as described in Example 1.

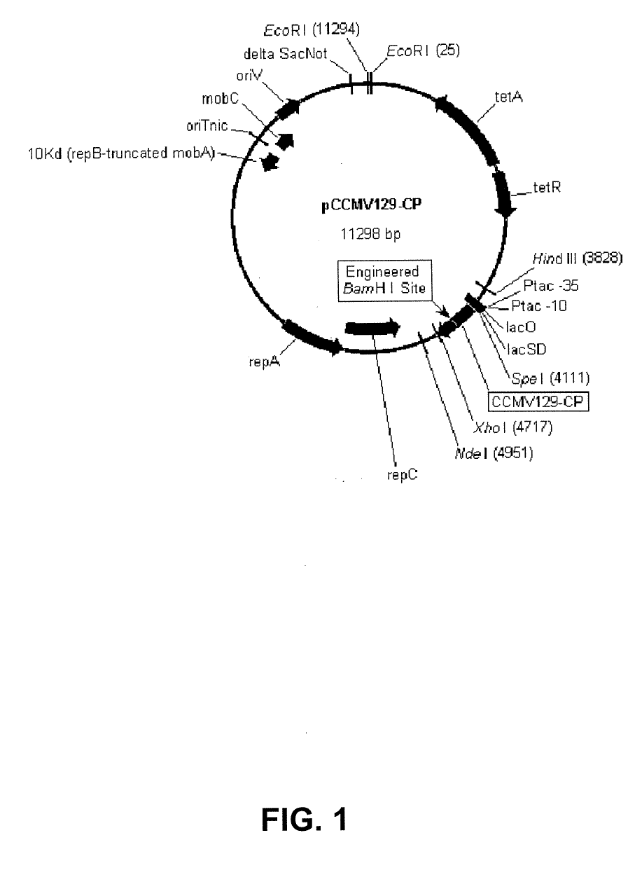

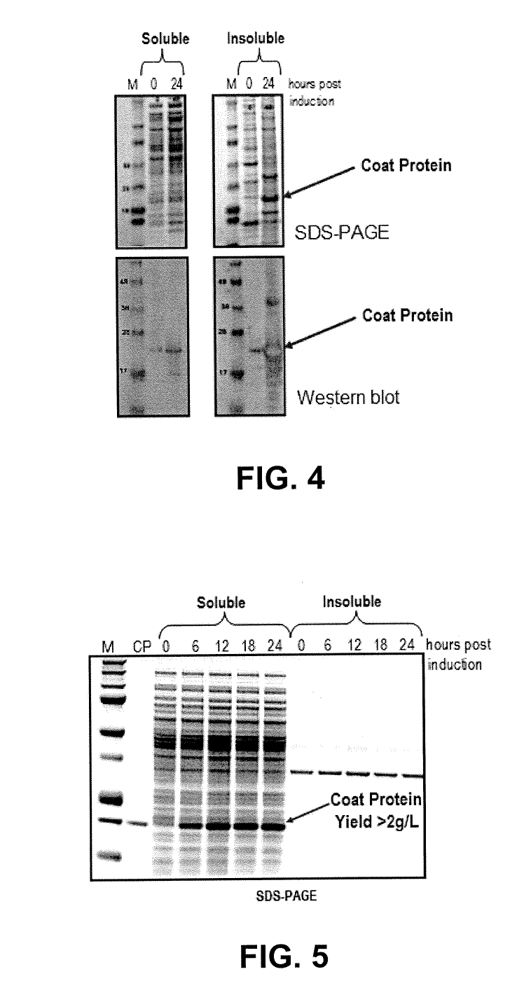

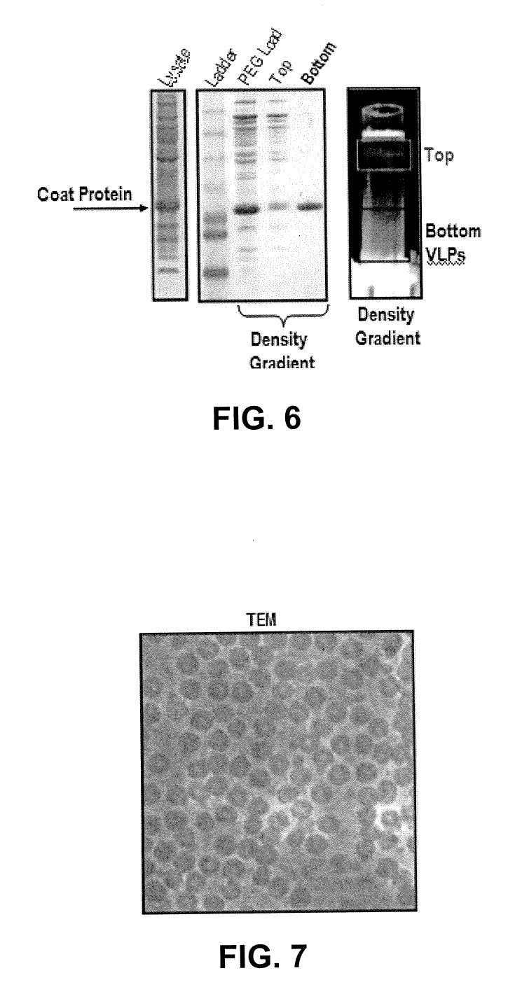

The hydrophilicity optimized CCMV coat protein was mostly soluble (FIG. 5) which is in direct contrast with expression of hydrophilicity unoptimized CCMV coat proteins (FIG. 4). The hydrophilicity unoptimized CCMV CP expression plasmid is shown in FIG. 1. The soluble, hydrophilicity optimized CCMV coat protein was purified by PEG precipitation and sucrose density gradient (see FIG. 6) and imaged by electron microscopy (see FIG. 7).

### Example 11

### Expression of Soluble Codon-Unoptimized CCMV-PA1 in P. fluorescens

CCMV129-PA1 has BamHI restriction sites (highlighted) flanking a PA1 insert: ATGTCTACAGTCGGAACAGGGAAGTTAACTCGTGCACAACGAAGGGCTGCGGCCCG TAAGAACAAGCGGAACACTCGTGTGGTCCAACCTGTTATTGTAGAACCCATCGCTTC AGGCCAAGGCAAGGCTATTAAAGCATGGACCGGTTACAGCGTATCGAAGTGGACCG CCTCTTGTGCGGCTGCCGAAGCTAAAGTAACCTCGGCTATAACTATCTCTCTCCCTA ATGAGCTATCGTCCGAAAGGAACAAGCAGCTCAAGGTAGGTAGAGTTTTATTATGG CTTGGGTTGCTTCCCAGTGTTAGTGGCACAGTGAAATCCTGTGTTACAGAGACGCAG ACTACTGCTGCTGCCTCCTTTCAGGTGGCATTAGCTGTGGCCGACAACGGGATCCTT AGTAATTCTCGTAAGAAACGTTCTACCTCTGCTGGCCCTACCGTGCCTGATCGTGAT AATGATGGCATTCCTGATGGGATCCTGTCGAAAGATGTTGTCGCTGCTATGTACCCC GAGGCGTTTAAGGGTATAACCCTTGAACAACTCACCGCGGATTTAACGATCTACTTG TACAGCAGTGCGGCTCTCACTGAGGGCGACGTCATCGTGCATTTGGAGGTTGAGCAT GTCAGACCTACGTTTGACGACTCTTTCACTCCGGTGTAT (SEQ ID NO:58).

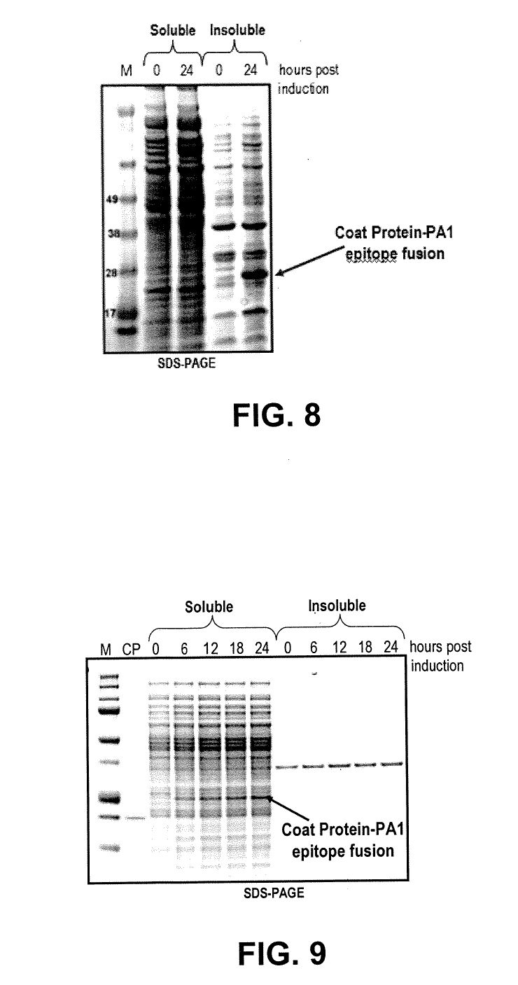

This construct yielded mostly insoluble CCMV-PA1 proteins (FIG. 8). The BamHI sites were removed by site-directed mutagenesis using the primers CCMV-PA1-nobam5-F (GCATTAGCTGTGGCCGACAACAGTAATTCTCGTAAGAAACG, SEQ ID NO:59) AND CCMV-PA1-nobam5-R (CGTTTCTTACGAGAATTACTGTTGTCGGC CACAGCTAATGC, SEQ ID NO:60). Site-directed mutagenesis reactions were carried out using Quikchange II-XL (Stratagene, TX) according to manufacturer's protocol. The P. fluorescens expression plasmid harboring codon-unoptimized, CCMV-PA1 served as template. Resulting plasmids were transformed into P. fluorescens strain DC454 (ΔpyrF RXF01414 (lsc)::lacIq1) by electroporation after purification with Micro Bio-spin 6. The deletion was verified by sequencing. The plasmid then served as template for the 3′ BamHI deletion using primers CCMV-PA1-nobam3-F (CGTGATAATGATGGCATTCCTGATTCGAAAGATGTTG TCGCTGC, SEQ ID NO:61) AND CCMV-PA1-nobam3-R (GCAGCGACAACATCTTTCGAA TCAGGAATGCCATCATTATCACG, SEQ ID NO:62).

The resulting plasmid was transformed into P. fluorescens DC454 as described above. The deletion was verified by sequencing.

### Protein Expression:

Single transformants were inoculated into 50 ml M9 Glucose media and grown overnight. P. fluorescens cultures of 3.0-5.0 OD600 were used to inoculate 200 mlDow's proprietary shake-flask media. Shake flask cultures flasks were incubated at 30° C. with 300 rpm shaking overnight. Overnight cultures of 15.0-20.0 OD600 were induced with 300 μM isopropyl-β-D-thiogalactopyranoside (IPTG). Cultures were harvested at 24 hours post induction. The soluble CCMV-PA1 coat protein expression is shown in FIG. 9.

### Example 12

### Expression of Soluble Codon-Unoptimized CCMV-PA4 in P. fluorescens

CCMV129-PA4 has BamHI restriction sites (highlighted) flanking PA4 insert: |ATGTCTACAGTCGGAACAGGGAAGTTAACTCGTGCACAACGAAGGGCTGCGGCCCG TAAGAACAAGCGGAACACTCGTGTGGTCCAACCTGTTATTGTAGAACCCATCGCTTC AGGCCAAGGCAAGGCTATTAAAGCATGGACCGGTTACAGCGTATCGAAGTGGACCG CCTCTTGTGCGGCTGCCGAAGCTAAAGTAACCTCGGCTATAACTATCTCTCTCCCTA ATGAGCTATCGTCCGAAAGGAACAAGCAGCTCAAGGTAGGTAGAGTTTTATTATGG CTTGGGTTGCTTCCCAGTGTTAGTGGCACAGTGAAATCCTGTGTTACAGAGACGCAG ACTACTGCTGCTGCCTCCTTTCAGGTGGCATTAGCTGTGGCCGACAACGGGATCCGT CAAGATGGCAAAACCTTCATTGATTTCAAAAAGTATAATGATAAACTCCCTCTCTAT ATTTCTAATCCTAATGGGATCCTGTCGAAAGATGTTGTCGCTGCTATGTACCCCGAG GCGTTTAAGGGTATAACCCTTGAACAACTCACCGCGGATTTAACGATCTACTTGTAC AGCAGTGCGGCTCTCACTGAGGGCGACGTCATCGTGCATTTGGAGGTTGAGCATGTC AGACCTACGTTTGACGACTCTTTCACTCCGGTGTAT|

This construct yielded mostly insoluble CCMV-PA4 proteins. The BamHI sites were removed to increase hydrophilicity by site-directed mutagenesis using primers CCMV-PA4-nobam5-F (GCATTAGCTGTGGCCGACAACCGTCAAGATGGCAAAACCTTC, SEQ ID NO:63) AND CCMV-PA4-nobam5-R (GAAGGTTTTGCCATCTTGACGGTTGTCGG CCACAGCTAATGC, SEQ ID NO:64). Site-directed mutagenesis reactions were carried out using Quikchange II-XL (Stratagene, TX) according to manufacturer's protocol using CCMV harboring codon-unoptimized, PA4 as template. Resulting plasmids with the 5′ BamHI restriction site deleted were transformed into P. fluorescens strain DC454 by electroporation after purification and then served as template for the 3′ BamHI deletion using the primer CCMV-PA4-nobam3-F (CTCTATATTTCTAATCCTAATTCGAAAGATGTTGTCGCTGC, SEQ ID NO:65) AND CCMV-PA4-nobam3-R (GCAGCGACAACATCTTTCGAATTAGGA TTAGAAATATAGAG, SEQ ID NO:66).

The resulting plasmid was transformed into P. fluorescens DC454 as described above. The deletion was verified by sequencing. Protein Expression was carried out as described in Example 1. The hydrophilicity optimized CCMV-PA4 coat protein was mostly soluble.

What is claimed is:

1. A method for the in vivo production of soluble assembled recombinant virus-like particles in a host cell including:
providing a host cell;
providing an isolated nucleic acid encoding a hydrophilicity-optimized coat protein-peptide fusion;
expressing the isolated nucleic acid in the host cell, wherein the expression in the cell provides for in vivo assembly of the hydrophilicity-optimized CP-fusion peptide into soluble virus-like particles; and
isolating the virus-like particles.

2. The method of claim 1, wherein providing an isolated nucleic acid encoding a hydrophilicity-optimized coat protein-peptide fusion comprises mixing, in vitro:
at least one first viral capsid fusion peptide comprising at least one antigenic peptide insert; and
at least one second viral capsid fusion peptide comprising at least one antigenic peptide insert, wherein the at least one second viral capsid fusion peptide comprises at least one antigenic peptide insert that is not present in the first viral capsid fusion peptide.

3. The method of claim 2, wherein the viral capsid of the first and/or second viral capsid fusion peptides are derived from the amino acid sequence of an icosahedral virus.

4. The method of claim 2, wherein the icosahedral virus is cowpea chlorotic mottle virus.

5. The method of claim 1, wherein the hydrophilicity-optimized coat protein-peptide is derived from the amino acid sequence of an icosahedral virus.

6. The method of claim 5, wherein the icosahedral virus is cowpea chlorotic mottle virus.

7. The method of claim 1, wherein the hydrophilicity-optimized coat protein-peptide is derived from SEQ ID NO:1.

8. The method of claim 1, wherein providing an isolated nucleic acid encoding a hydrophilicity-optimized coat protein-peptide fusion comprises providing an isolated nucleic acid encoding a coat protein peptide having modified amino acids in the position 63 and 129 insertion sites of the coat protein construct.

9. The method of claim 8, wherein the isolated nucleic acid encoding a coat protein peptide having amino acids in the position 63 and 129 insertion sites of the coat protein construct are modified via removal of the amino acids.

10. The method of claim 9, wherein the amino acids that are removed in the position 63 and 129 insertion sites comprise arginine, tryptophan, glycine, isoleucine, and leucine.

11. The method of claim 8, wherein the coat protein comprises a cowpea chlorotic mottle virus coat protein.

12. The method of claim 8, wherein the isolated nucleic acid encoding a coat protein peptide having amino acids in the position 63 and 129 insertion sites of the coat protein construct are modified by site directed mutagenesis.

13. The method of claim 8, wherein the isolated nucleic acid encoding a coat protein peptide having amino acids in the position 63 and 129 insertion sites of the coat protein construct are modified using splicing by overlap extension-based technology.

14. The method of claim 8, wherein the isolated nucleic acid encoding a coat protein peptide having amino acids in the position 63 and 129 insertion sites of the coat protein construct are modified by replacing a codon encoding an amino acid of low hydrophilicity with an amino acid having a higher hydrophilicity value.

15. The method of claim 1, further comprising cleaving the fusion peptide product to separate the recombinant polypeptide from the capsid protein.

16. The method of claim 1, wherein the host cell is a Pseudomonas cell.

17. The method of claim 16, wherein the host cell is Pseudomonas fluorescens.

18. A method for the in vivo production of soluble assembled recombinant virus-like particles in a host cell including:
providing a Pseudomonas host cell;
providing an isolated nucleic acid encoding a coat protein peptide having modified amino acids in the position 63 and 129 insertion sites of a Cowpea Chlorotic Mottle Virus (CCMV) coat protein construct to form a hydrophilicity-optimized CP-fusion peptide;
expressing the isolated nucleic acid in the Pseudomonas host cell, wherein the expression in the cell provides for in vivo assembly of the hydrophilicity-optimized CP-fusion peptide into soluble virus-like particles; and
isolating the virus-like particles.

19. The method of claim 18, wherein the host cell is Pseudomonas fluorescens.

20. The method of claim 18, wherein the isolated nucleic acid encoding a coat protein peptide having amino acids in the position 63 and 129 insertion sites of the coat protein construct are modified by site directed mutagenesis.

21. The method of claim 18, wherein the isolated nucleic acid encoding a coat protein peptide having amino acids in the position 63 and 129 insertion sites of the coat protein construct are modified using splicing by overlap extension-based technology.

22. The method of claim 18, wherein the isolated nucleic acid encoding a coat protein peptide having amino acids in the position 63 and 129 insertion sites of the coat protein construct are modified via removal of the amino acids.

23. A virus-like particle comprising an isolated nucleic acid encoding a coat protein peptide having modified amino acids in the position 63 and 129 insertion sites of a coat protein construct.

24. The virus-like particle of claim 23, wherein the amino acid sequence is derived from the amino acid sequence of an icosahedral virus.

25. The method of claim 24, wherein the icosahedral virus is cowpea chlorotic mottle virus.

26. The method of claim 23, wherein the isolated nucleic acid comprise a coat protein peptide having amino acids in the position 63 and 129 insertion sites of a coat protein construct removed.

27. The method of claim 26, wherein the amino acids that are removed in the position 63 and 129 insertion sites comprise arginine, tryptophan, glycine, isoleucine, and leucine.
